(19) United States
(12) Patent Application Publication
(10) Pub. No.: US 2026/0069555 A1
(43) Pub. Date: Mar. 12, 2026

(54) Compositions and Methods for Modulating the Effect of Beta-Blocker Activity in a Subject

(71) Applicant: Duke University, Durham, NC (US)

(72) Inventors: Robert Lefkowitz, Durham, NC (US); Howard Rockman, Durham, NC (US); Biswaranjan Pani, Durham, NC (US); Jialu Wang, Durham, NC (US); Alem Kahsai, Durham, NC (US)

(21) Appl. No.: 18/707,971

(22) PCT Filed: Nov. 15, 2022
(86) PCT No.: PCT/US2022/079847

§ 371 Date: May 7, 2024

Related U.S. Application Data
(60) Provisional application No. 63/280,194, filed on Nov. 17, 2021.

(51) Int. Cl. A61K 31/18; A61K 31/403; A61P 9/10

(52) U.S. Cl. CPC A61K 31/18; A61K 31/403; A61P 9/10

(57) ABSTRACT

The present disclosure provides compositions that provide positive allosteric modulator compounds of the beta-adrenergic receptor in combination with a beta-arrestin-biased beta-blocker, such as carvedilol, for the treatment of cardiovascular diseases and disorders, such as hypertension and heart failure, wherein the effectiveness of the beta-blocker is enhanced by positively augmenting carvedilol-stimulated cellular responses.

### CROSS-RELATION TO RELATED APPLICATIONS

This application claims benefit of U.S. Provisional Patent Application No. 63/280,194, filed Nov. 17, 2021, the contents of which are herein incorporated in their entirety by reference.

### STATEMENT AS TO FEDERALLY SPONSORED RESEARCH

This invention was made with Government support under Federal Grant Nos. R01HL056687, R01HL075443, AND R01HL16037 awarded by the NIH. The Federal Government has certain rights to this invention.

### TECHNICAL FIELD

The present invention generally relates to the technical fields of tumor biology, oncology, immunology, and medicine.

### BACKGROUND

β1 adrenergic receptors (β1ARs) are key regulators of heart rate and myocardial contractility and a common therapeutic target for the treatment of cardiac diseases such as hypertension and heart failure. As a member of the G protein-coupled receptor (GPCR) family, β1ARs primarily transduce signals through stimulatory guanine nucleotide-binding proteins (Gas proteins). Upon receptor activation by endogenous ligands, such as the catecholamines epinephrine or norepinephrine, β1ARs couple to Gs proteins to activate the effector enzyme adenylyl cyclase to catalyze the generation of second messenger cAMP, leading to downstream signaling and a diverse array of cellular and physiological responses. Sustained Gs signaling, activated by chronic neurohumoral stimulation of the β1AR can lead to lethal cardiac arrhythmias and deleterious maladaptive cardiac remodeling (Rockman et al., 2002). Therefore, β-blockers that prevent excessive β1AR activation by blunting catecholamine-stimulated downstream Gs signaling, such as carvedilol, metoprolol and bisoprolol are widely used in the treatment of heart failure. However, despite the fact that β-blockers effectively decrease morbidity and mortality in heart failure, there is a large variation among patients in the level of responsiveness to these drugs.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The inventors have recently discovered a positive allosteric activity of the beta-adrenergic receptor modulator compound-6 (see, U.S. patent application Ser. No. 16/269,877, filed Feb. 7, 2019 and entitled “System and Method for Homogenous GPCR Phosphorylation and Identification of Beta-2 Andrenergic Receptor Positive Allosteric Modulators,” the contents of which are hereby incorporated by reference in its entirety) for the FDA approved beta-blocker carvedilol at both beta1-and beta2-adrenergic receptors. Unlike conventional beta-blockers that are therapeutically used for treating heart problems, carvedilol displays unique pharmacological properties. Carvedilol exclusively stimulates beta-arrestin-mediated (cardioprotective) signaling pathways without eliciting G protein-dependent cellular events. The inventors have extensively characterized the pharmacology between compound-6 and carvedilol and have also assessed the potential physiological implications of this phenomenon. The present disclosure is based, in part, on the discovery by the inventors that that compound-6 positively augments carvedilol-stimulated cellular responses and elicits beta-adrenergic receptor-mediated cardioprotection in a mouse model of cardiac injury.

Accordingly, one aspect of the present disclosure provides a method of enhancing the effectiveness of a β-blocker being administered to a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the effectiveness of the β-blocker is enhanced.

Another aspect of the present disclosure provides a method of treating and/or preventing high blood pressure in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the high blood pressure is treated and/or prevented in the subject.

Another aspect of the present disclosure provides a method of treating and/or preventing heart disease in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the heart disease is treated and/or prevented in the subject.

Another aspect of the present disclosure provides a method of treating and/or preventing migraines in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the migraine is treated and/or prevented in the subject.

Another aspect of the present disclosure provides a method of treating and/or preventing glaucoma in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the glaucoma is treated and/or prevented in the subject.

Another aspect of the present disclosure provides a method of treating and/or preventing anxiety in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the anxiety is treated and/or prevented in the subject.

Another aspect of the present disclosure provides a method of treating and/or preventing overactive thyroid in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the overactive thyroid is treated and/or prevented in the subject.

Another aspect of the present disclosure provides a method of treating and/or preventing essential tremor in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the essential tremor is treated and/or prevented in the subject.

Another aspect of the present disclosure provides a method of enhancing the effectiveness of a beta-arrestin-biased β-blocker being administered to a subject, the method comprising administering to the subject a therapeutically effective amount of a compound according to Formula (I):

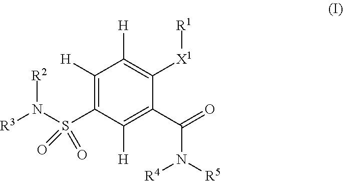

wherein:
R₁ is an aryl group optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆ alkyl)₂ —OC₃₋₆cycloalkyl, —NHC₃₋₆cycloalkyl, —N(C₁₋₆alkyl)(C₃₋₆cycloalkyl), and —N(C₃₋₆cycloalkyl)₂, and optionally the aryl is phenyl wherein two substituents join to form a 5- to 7-membered non-aromatic fused ring containing 1-2 heteroatom groups selected from NR^{1a} and O;
X₁ is O, N(H), N(C₁₋₄alkyl), S, S(O), S(O)₂, C(O), or CR^{1b}R^{1c};
R^{1a} is H or C₁₋₄alkyl;
R^{1b} and Ric are each independently hydrogen or C₁₋₄alkyl, or R^{1b} and Ric together with the carbon to which they are attached form a C₃₋₆cycloalkyl ring;
R² is hydrogen, C₁₋₆alkyl, or C₃₋₇cycloalkyl;
R³ is hydrogen, C₁₋₆alkyl, C₃₋₇cycloalkyl, or aryl, the aryl being optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂; alternatively, R² and R³ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R⁴ is hydrogen, C₁₋₆alkyl, or C₃₋₇cycloalkyl;
R⁵ is CHR^{5a}R^{5b}; alternatively, R⁴ and R⁵ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R^{5a} is aryl or —C₁₋₃alkylene-aryl, wherein each aryl in R^{5a} is optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆ alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂;
R^{5b} is X² or —C₁₋₃alkylene-X²; and
X² is —CN, —C(O)OH, —C(O)OC₁₋₄alkyl, —C(O)NH₂, —C(O)NHC₁₋₄alkyl, —C(O)N(C₁₋₄alkyl)₂, —SO₂NH₂, —SO₂NHC₁₋₄alkyl, or —SO₂N(C₁₋₄alkyl)₂. (Cmpd 6), or a pharmaceutically acceptable salt, solvate, hydrate, prodrug, or derivative thereof, or a pharmaceutical composition thereof, and
a β-arrestin-biased β-blocker such that the effectiveness of the β-arrestin-biased p-blocker is enhanced.

Another aspect of the present disclosure provides a method of treating and/or preventing high blood pressure or heart disease in a subject, the method comprising administering a therapeutically effective amount of a compound according to Formula (I) and a β-arrestin-biased β-blocker such that the high blood pressure or heart disease is treated and/or prevented in the subject.

Another aspect of the present disclosure provides a pharmaceutical composition comprising a compound according to Formula (I) or a pharmaceutically acceptable salt, solvate, hydrate, prodrug, or derivative thereof, and a β-arrestin-biased β-blocker or a pharmaceutically acceptable salt, solvate, hydrate, prodrug, or derivative thereof.

Another aspect of the present disclosure provides all that is described and illustrated herein.

### BRIEF DESCRIPTION OF THE DRAWINGS

The foregoing aspects and other features of the disclosure are explained in the following description, taken in connection with the accompanying drawings, herein:

FIGS. 1A-1G are graphic representations of data showing Cmpd-6 is positively cooperative with the β-blocker Carvedilol in accordance with one embodiment of the present disclosure. FIGS. 1A-1C: Radioligand competition binding showing the displacement of 125I-CYP ([¹²⁵I]-Cyanopindolol) by the cold competitor's epinephrine (FIG. 1A), carazolol (FIG. 1i), and carvedilol (FIG. 1C), respectively, at the β2AR in HDL. Data showing left-shifts in epinephrine (diamond) and carvedilol (circle) competition curves indicate positive cooperativity with Cmpd-6. Chemical structures of competing ligands are show on top of respective data panel (FIGS. 1A-1C). Points on the curves represent normalized cpm values from three independent experiments ±SD. FIG. 1D: Bar graph showing Cmpd-6-mediated affinity shifts (K-shifts), reported as the difference in Log IC50 (Cmpd-6 vs DMSO) for a diverse panel of β-ligands; full agonists (grey), partial agonists (light grey), and antagonists (medium grey). Data represent shifts in Log IC₅₀(Log K-shifts) values derived from three to six independent experiments ±SD. FIG. 1E: Correlation plot showing comparison of affinity-shifts (Log K-shifts) for a panel of P-ligands mediated by Nb80 and Cmpd-6 (this study). Dashed lines around the line of correlation (solid) represent the 95% confidence interval. Carvedilol is the one outlier ligand that is cooperative with only Cmpd-6 but not Nb80. FIG. 1F: Cmpd-6 dose response curves obtained by displacement of ¹²⁵I-CYP by the cold carvedilol. FIG. 1G: Curve showing shifts in Log IC50 (ΔLog-IC50) of carvedilol mediated by Cmpd-6 dose response and is derived from data in (FIG. 1F). Points on the curves represent normalized cpm (FIG. 1F) and ΔLog-IC50 (FIG. 1G) values from four independent experiments ±SD. Statistical comparisons were done using one-way ANOVA with Bonferroni's post hoc test, ***P<0.001, ns, not significant.

FIGS. 2A-2G are graphic representations of data showing Cmpd-6 and β-arrestin1-mediated high-affinity binding of Carvedilol in accordance with one embodiment of the present disclosure. FIGS. 2A-22D show radioligand competition binding showing the displacement of ¹²⁵I-CYP ([¹²⁵I]-Cyanopindolol) by the cold competitors carvedilol (FIGS. 2A and 2B) and carazolol (FIGS. 2C and 2D), respectively, at the β2V2RpP in HDL. Cmpd-6 is shown to display cooperative effects with βarr1(FIG. 2A), but not with Gs (FIG. 2B), in promoting high-affinity left-shifts of carvedilol competition curves. No such cooperativity is observed in carazolol competition curves. FIGS. 2E and 2F: Bar graph showing comparison of respective ligand affinity-shifts (Log K-shifts) mediated by cmpd-6. Data shown in FIGS. 2a-2D) and FIGS. 2E and 2F represent values obtained from three independent experiments ±SD. FIG. 2G: Bar graph showing the direct high-affinity binding of 3H-Carvedilol (³H-Carv) to β2ARpP in HDL. Compared to DMSO control, cmpd-6 alone and together with β-arrestin1 (βarr-1), but not Gs, is shown to potentiate ³H-Carvedilol binding. Data shown in the bar graphs represent mean receptor binding values obtained from four independent experiments ±SD. Statistical comparisons were made by two-way ANOVA followed by Bonferroni post hoc test. ** indicate P<0.01, *** indicate P<0.001, ns, not significant.

FIGS. 3A-3E are representations of data showing Cmpd-6 augments cellular activity of carvedilol at the β2AR in accordance with one embodiment of the present disclosure. FIG. 3A: HEK293 cells stably expressing GloSensor were pretreated with either vehicle (dimethyl sulfoxide, DMSO) alone or cmpd-6 (30 μM) for 15-20 min. The extent of cAMP generation by endogenously expressed β2AR was subsequently measured after stimulation of the cells with either epinephrine (EPI) or carvedilol (CARV) for 5-10 min in a dose-dependent manner. Values were normalized to the maximal level of EPI-induced activity in the vehicle (0.3% DMSO) control, expressed as a percentage, and represents mean±SD. FIGS. 3B and 3C: Cells were pretreated with either vehicle DMSO alone (FIG. 3B) or Cmpd-6 (FIG. 3C) for 15-20 min and inhibition of 1 μM EPI-stimulated cAMP generation was monitored in the presence of a dose of either carvedilol or metoprolol. Values were normalized to the uninhibited EPI signal in the DMSO control. Dose-dependent curve fits were generated with data points obtained from three-four independent experiments done in duplicate. FIG. 3D: HEK293 cells stably expressing the β2AR were serum-starved overnight and subsequently pretreated with either vehicle (DMSO) alone or Cmpd-6 at 5 μM for 15-20 min. The cells were then stimulated with carvedilol (CARV) for 5 min in a dose-dependent manner. ERK phosphorylation (p-ERK) and total ERK expression (t-ERK) in each sample were visualized by immunoblotting as described. FIG. 3E: Each of the p-ERK and t-ERK bands in the immunoblot was quantified as described, and the extent of ERK phosphorylation was determined through dividing the p-ERK signal by the t-ERK. Each data point was expressed as percent of the maximal response in the vehicle-treated control cells and represents the mean±SD from five independent experiments. Dose-response curves and EC50 values between vehicle (DMSO) and Cmpd-6-treated samples were obtained and by using GraphPad Prism. Statistical significance for the difference in Log Kᵢ values (carvedilol vs metoprolol) between vehicle (DMSO)- and cmpd-6-treated curve fits (P<0.001) was determined by two-way ANOVA. *** indicate P<0.001.

FIGS. 4A-4AH are representations of data showing that Cmpd-6 augments carvedilol-stimulated β2AR internalization in accordance with one embodiment of the present disclosure. FIG. 4A-4C: Cell surface ELISA showing the cooperative effects of cmpd-6 on loss of cell surface β2AR following a dose response of epinephrine (FIG. 4A), carvedilol (FIG. 4B), and ICI-118551 (FIG. 4C), normalized mean±SD, n=5. FIG. 4D: Bar graph showing mean difference in Log EC₅₀ (vs. DMSO control) for epinephrine (dark grey, n=5) and carvedilol (light grey, n=5) in presence of cmpd-6. FIGS. 4E-4G: Representative confocal images showing cmpd-6 mediated potentiation of lysosomal targeting of the β2AR upon stimulation with carvedilol. Scale bar on respective images is 10μ. FIG. 4H: Bar graph showing quantification of β2AR-YFP (light linear areas in images of FIGS. 4E-4G) colocalization with lysosomes (light points in images of FIGS. 4E-4G) and is expressed as colocalization index±SD as described in the methods. Statistical comparisons were made using one-way ANOVA with Bonferroni's post hoc tests. **P<0.01, ***P<0.001.

FIGS. 5A-5G are graphic and schematic representations of data showing Cmpd-6 analog A9 displays carvedilol specific cooperativity at the β2AR in accordance with one embodiment of the present disclosure. FIGS. 5A and 5B: Bar graph showing affinity shifts (Log K-shifts) of epinephrine (FIG. 5A) and carvedilol (FIG. 5B) mediated by cmpd-6 or its analogs (A1-A12) compared to DMSO control. FIG. 5C: Chemical structures of cmpd-6 and its analog A9 highlighting the modified R2 moiety in the analog. FIG. 5D: Bar graph showing the allosteric cooperative effects of A9 on affinity shifts of a panel of P-ligands (agonists and antagonists). A9 shows positive cooperativity only with carvedilol compared to other ligands tested. Bar graphs in FIGS. 5A, 5B and 5C show mean Log K-shifts ±SD obtained from three-five independent experiments. FIG. 5E: Curve showing affinity shifts in Log IC₅₀(Log K-Shift) for carvedilol mediated by a dose of A9. Points on the curves represent Log K-Shift values derived from three independent competition binding experiments ±SD. FIG. 5F: Bar graph showing binding of ³⁵S-GTPγS to heterotrimeric Gs following epinephrine stimulation of the β2AR in HDL. Data for respective conditions (n=4) are normalized to basal (unstimulated) binding of ³⁵S-GTPγS. FIG. 5G: Curves showing the effect of cmpd-6 and A9 on isoproterenol (ISO) stimulated cAMP generation. Cells were pretreated for 15-20 min with either cmpd-6 or A9 (30 μM) and then stimulated with a dose of ISO. The amount of cAMP production by endogenously expressed β2AR was measured at 10 min after ISO stimulation. Curve fits were generated in GraphPad Prism with data points obtained five independent experiments done in duplicate. Each data point was normalized to the maximal level of ISO-induced activity in the vehicle (DMSO) control, expressed as a percentage, and represents mean±SD. Statistical comparisons were made using one-way ANOVA with Bonferroni's post hoc tests. *P<0.05, **P<0.01, ***P<0.001, ns, not significant.

FIG. 6 is a array listing orthosteric P-ligands used in the studies herein. Chemical structures and molecular weights (MW) of ligands were obtained from CheMBL database.

FIGS. 7A and 7B are graphic representations of data showing positive cooperativity between cmpd-6 and carvedilol at the β1AR. FIG. 7A: Radioligand competition binding showing the displacement of ¹²⁵I-CYP ([¹²⁵I]-Cyanopindolol) by a serial dilution of cold carvedilol at the β1AR in HDL (high density lipoprotein particles or nanodiscs). Cmpd-6 results in shifting the carvedilol (open circles) competition curve to higher affinity compared to DMSO control (closed circles). FIG. 7B: Curve showing cmpd-6 dose-dependent high-affinity shifts for carvedilol. Log K-shift values for carvedilol were derived from ¹²⁵I-CYP competition bindings at the R 1AR done without (DMSO) or with dose of cmpd-6. Data show are normalized cpm (FIG. 7A) and Log K-shift values (FIG. 7B) obtained from four independent experiments expressed as mean±SD. Statistical comparisons for data in FIG. 7A was done by one-way ANOVA with Bonferroni's post hoc test, ***P<0.001.

FIGS. 8A-8F are graphic representations of data showing positive cooperativity between cmpd-6 and carvedilol at β2AR membranes. ¹²⁵I-CYP competition radioligand binding at β2V2R expressing U2OS (FIGS. 8A-8C) and β2AR-YFP expressing HEK293 (FIGS. 8D-8F) cell membranes. Left-shifted curves indicate positive allosteric cooperativity of cmpd-6 (vs. DMSO) with epinephrine (FIGS. 8A, FIG. 8D), carvedilol (FIGS. 8C, FIGS. 8F) but not with carazolol (FIGS. 8B, FIGS. 8E). Data shown are normalized cpm values obtained from three independent experiments expressed as mean±SD. Statistical comparisons were done using one-way ANOVA with Bonferroni's post hoc test, ***P<0.001, ns, not significant.

FIG. 9 is a graphic representation showing the effect of Cmpd-6 on carvedilol stimulated endocytosis of β2V2R. HEK293cells expressing the β2V2R were treated with a dose of carvedilol for 16 h in the absence (DMSO, black circles) or presence of cmpd-6 (grey squares). Curve fits represent data points obtained from six independent experiments done in duplicate. Each data point was first normalized to the basal signal and then to the maximal level of carvedilol-induced activity in controls (DMSO), expressed as a percentage, and represents mean±SD. Fold changes in EC50 and % Bmax values are indicate by arrows (grey). Statistical comparisons between vehicle (DMSO)- and Cmpd-6-treated curve fits (P<0.001) was determined by two-way ANOVA.

FIGS. 10A-10K are graphic representations of data showing Cmpd-6 selectively potentiates the binding affinity of carvedilol for the β1AR in accordance with one embodiment of the present disclosure. FIG. A: Cmpd-6 led to a leftward shift of the carvedilol competition binding curve against ¹²⁵I-cyanopindolol (CYP) to the β1AR, indicating the increased receptor binding affinity of carvedilol. β1ARs reconstituted in high-density lipoprotein particles were incubated with vehicle (DMSO) or 25 μM Cmpd-6, indicated concentration of carvedilol and 60 μM ¹²⁵I-CYP. Values were normalized to the percentage of maximal ¹²⁵I-CYP binding level. FIG. 10B: Cmpd-6 modestly enhanced the binding affinity of isoproterenol for the β1AR by 2-fold. FIGS. 10C-10J: Cmpd-6 had minimal effect on the β1AR binding affinity of a panel of agonists and antagonists tested. FIG. 10K: Cmpd-6-led leftward shift of ligands competition binding curves, shown as the difference of log IC₅₀ values of the vehicle- and Cmpd-6-treated groups for each ligand. Cmpd-6 showed strong cooperativity with carvedilol (light grey bar), a modest positive allosteric modulator (PAM) activity on full agonists (black bars), no positive modulation on other antagonists tested (medium grey bars). Data represent the mean±S.D. for three independent experiments. Competition binding curves were generated with GraphPad Prism. The log IC₅₀ values, shown in the legends of FIGS. 10A-10C and represented in FIG. 10K, were calculated from competition binding curves with GraphPad Prism. Statistical comparisons were performed using two-way repeated (related)-measures ANOVA with Sidak correction for multiple comparisons (FIGS. 10A-10C) or two-tailed Student's t-test for each ligand testing the mean vs. the null hypothesis that the shift in Log IC₅₀ is 0 (FIG. 10K). *P<0.05, **P<0.01.

FIGS. 11A-11H show data showing that Cmpd-6 potentiates carvedilol-stimulated β-arrestin-mediated, but not Gs protein-mediated β1AR signaling in accordance with one embodiment of the present disclosure. FIG. 11A: shows a schematic illustrating β1AR-mediated Gs activation monitored with GloSensor cAMP assay (Promega), a luciferase-based biosensor to monitor cAMP level. FIG. 11B: Cmpd-6 had no effect on isoproterenol- or carvedilol-induced β1AR-mediated Gs activation. HEK293 cells transiently transfected with β1ARs and GloSensor were pretreated with vehicle (DMSO) or 30 μM Cmpd-6 for 20 min, together with 100 nM ICI118,551, the highly selective β2AR antagonist, to block the activation of endogenous β2ARs. Cells were then stimulated with serial doses of isoproterenol or carvedilol for 5 min. The luminescence values were normalized to the percentage of maximal isoproterenol-induced level in the vehicle-treated group. FIG. 11C: shows a schematic illustrating β1AR-mediated EGFR transactivation determined by monitoring EGFR endocytosis with BRET assay with EGFR_RlucII and endosomal-located rGFP_FYVE. FIG. 11D: Cmpd-6 potentiated carvedilol-stimulated β1AR-mediated EGFR transactivation, indicated by a leftward shift of the dose response curve. HEK293 cells stably expressing β1ARs were transfected with EGFR-RlucII and rGFP-FYVE constructs. Cells were pretreated with vehicle (DMSO) or 30 μM Cmpd-6 for 20 min, then stimulated with indicated concentrations of carvedilol for 25 min before BRET measurement. The change in BRET ratio (emission ratio of RlucII to rGFP) is expressed as the difference between ligand-stimulated and unstimulated samples. FIGS. 10E and10F: Carvedilol-induced β1AR activation of ERK is mediated through β-arrestins. Wild type (WT) or β-arrestin1/2 knockout (β-arr1/2 KO) HEK293 cells transiently transfected with β1ARs were stimulated with serial concentrations of carvedilol for 5 min. Data is calculated as the ratio of phosphorylated ERK (pERK) to total ERK (tERK), then normalized as percentage of the maximum value in the wide type group. FIGS. 11G and 11H: Cmpd-6 led to a leftward shift of carvedilol dose response curve on β1AR-mediated ERK activation, indicating its PAM activity on this β-arrestin-dependent signaling. HEK293 cells stably expressing β1ARs were pretreated with vehicle or 30 μM Cmpd-6 for 20 min, then stimulated with indicated concentration of carvedilol for 5 min. The pERK/tERK ratio is normalized to the maximum value in the vehicle-treated group. Data represent the mean±S.D. for four to eight independent experiments as marked on the figure. Error bars in some data points in FIG. 11B are not visualizable because the error bar is shorter than the size of the symbol. Dose response curves and log EC₅₀ values shown in legends were generated with GraphPad Prism. Statistical comparisons were performed using two-way repeated (related)-measures ANOVA with Sidak correction for multiple comparisons. *P<0.05, **P<0.01, ****P<0.0001.

FIGS. 12A-12G show data showing that Cmpd-6 shows no PAM activity on β1AR-mediated ERK stimulated by a comprehensive panel of ligands. HEK293 cells stably expressing β1ARs were pretreated with vehicle or 30 μM Cmpd-6 for 20 min, then stimulated with serial concentrations of indicated ligands for 5 min. A panel of agonists (FIGS. 12A-12D) and β-blockers (FIGS. 12E-12G) dose-dependently induced β1AR-mediated ERK phosphorylation. Cmpd-6 pretreatment showed no effect on these ligands. Data is presented as pERK/tERK ratios normalized to the maximum value in the vehicle group. Data represent the mean±S.D. for three independent experiments. Dose response curves and log EC₅₀ values shown in legends were generated with GraphPad Prism. Statistical comparisons were performed using two-way repeated (related)-measures ANOVA with Sidak correction for multiple comparisons and showed no difference between vehicle- and Cmpd-6-treated groups for each ligand.

FIGS. 13A-13J show data showing that Cmpd-6 modestly enhances the inhibitory effect of carvedilol on catecholamine-induced hemodynamics in mouse hearts in accordance with one embodiment of the present disclosure. FIGS. 13A and 13B: Representative traces of hemodynamic measurements in mouse. A high fidelity micromanometer catheter connected with a pressure transducer was inserted retrograde into left ventricle to monitor blood pressure. Serial doses of isoproteronol (50, 500, 1000, 5000 pg) were injected intravenously at 45 sec intervals. FIG. 13A: Hemodynamic was monitored continuously and recorded between 35 to 45 sec after each injection when steady state was reached. FIG. 13B: expanded view of cardiac cycles, enlarged on X-axis (time). FIGS. 13C-13F: Carvedilol dose-dependently blocks isopreteronol-induced increase in heart rate and contractility (indicated by dP/dt max). Wild-type C57BL/6J mice were treated with vehicle or indicated doses of carvedilol (1, 5, 20 mg/kg/day) for 3 days with Alzet osmotic pumps before the isoproterenol-induced hemodynamic study. FIGS. 13G-13J: Cmpd-6 led to a modest enhancement of the inhibitory effect of carvedilol. Mice were treated with vehicle, carvedilol (1 or 20 mg/kg/day), or combination of carvedilol (1 mg/kg/day) and Cmpd-6 (5 mg/kg/day) for 3 days with Alzet osmotic pump. Hemodynamics were monitored and calculated using LabChart8 software. Data represent the mean±S.D. for four or five animals as marked on the figure. Data of each drug-treated group was compared to the vehicle-treated group using two-way repeated (related)-measures ANOVA with Sidak correction for multiple comparisons. *P<0.05, **P<0.01, ***P<0.0001 interaction vs. vehicle group.

FIGS. 14A-14E show data showing that Cmpd-6 potentiates the β-arrestin-dependent in vivo protective effect of carvedilol against ischemia/reperfusion injury-induced myocardium apoptosis. FIG. 14A: Scheme of the study. Mice were treated with carvedilol and Cmpd-6, individually or in combination, for 3 days with Alzet osmotic pumps. The left anterior descending (LAD) coronary artery was ligated for 45 min to produce cardiac ischemia, then released to allow blood flow restoration for 45 min. Level of apoptosis was assessed with TUNEL staining. FIGS. 14B and 14C: Carvedilol diminishes the ischemia/reperfusion-induced apoptosis in hearts, and co-administration of Cmpd-6 enhances this protective effect. Wild-type C57BL/6J mice were treated with vehicle, Cmpd-6 (5 mg/kg/day), carvedilol (1 mg/kg/day), or combination of both compounds for 3 days with Alzet osmotic pumps before the ischemia/reperfusion procedure was performed. FIGS. 14D and 14E: The anti-apoptotic effect of carvedilol is abolished in mice with cardiomyocyte-specific deletion of β-arrestins. The β-arrestin1/2 cardiac knockout animals (DMyHC Cre:Arrblflox/flox/Arrb2flox/flox) were treated with vehicle or carvedilol (1 or 20 mg/kg/day) for 3 days before the ischemia/reperfusion procedure was performed. Data represent the mean±S.D. for five to ten animals as marked on the figure. Statistical comparisons were performed using one-way ANOVA with Tukey correction for multiple comparisons.

FIG. 15 shows the chemical structure of Compound-6 (Cmpd-6).

FIGS. 16A and 16B are graphic representations of data showing that Cmpd-6 cooperates with carvedilol on both β1ARs and β2ARs. FIG. 16A: Cmpd-6-induced change in affinity of full agonists (isoproterenol, epinephrine, and norepinephrine), partial agonists (salmeterol, zinterol, procateral, and clenbuteral) and antagonists (carvedilol-labetalol) to β1ARs, plotted as the change in ligand log IC50 values in radioligand competition binding assays. Data represent the mean±S.D. for three independent experiments. FIG. 16B: Comparison of Cmpd-6-induced ligand affinity change at β1ARs (shown in FIG. 16A) and β2ARs (shown in FIG. 1D, herein). Ligands tested at both receptors are plotted here. The log IC50 values were calculated from competition binding curves with GraphPad Prism.

FIGS. 17A-17E show data relating to an EGFR transactivation assay. FIG. 17A: EGF induces a robust EGFR endocytosis, which is blocked by the EGFR inhibitor AG1478. The β1AR stably expressing HEK293 cells were transfected with EGFR-RLucII and rGFP-FYVE. Cells were pretreated with vehicle or 10 μM AG1478 for 20 min, then stimulated with serial concentrations of EGF for 25 min. Data are expressed as the difference of BRET ratio (emission ratio of RlucII to rGFP) between EGF-stimulated and unstimulated samples. FIGS. 17B-17D: The β1AR-mediated EGFR transactivation is diminished by the siRNA knockdown of β-arrestins. The β1AR stable cells were transfected with EGFR-RLucII, rGFP-FYVE, together with control siRNA or β-arrestin1/2 siRNA. Cells were stimulated with serial concentration of isoproterenol or carvedilol for 25 min. Representative blot shows the siRNA knockdown of β-arrestins. FIG. 17E: Cmpd-6 shows no positive cooperativity on isoproterenol-induced β1AR-mediated EGFR transactivation. Cells were pretreated with vehicle or 30 μM Cmpd-6 for 20 min, then stimulated with indicated doses of isoproterenol for 25 min. Data represent the mean±S.D. for three to eight independent experiments as marked on the figure. Statistical comparisons were performed using two-way repeated (related)-measures ANOVA with Sidak correction for multiple comparisons. *P<0.05, **P<0.01, ***P<0.001, ****P<0.0001.

FIGS. 18A-18H are graphic representations of data showing the effect of Cmpd-6 on ligand-stimulated β1AR-mediated ERK activation. Cmpd-6 leads to a leftward shift of the carvedilol dose response curve on ERK activation (FIG. 18A: vehicle: log EC50=−7.06 [−7.37 to −6.77], Cmpd-6: log EC50=−8.35 [−8.75 to −7.96]). FIGS. 18B-18H: Same data sets as in FIG. 11H and FIGS. 12A-12G, presented as fold over non-stimulated (NS) sample of each treatment. Data represent the mean±S.D. for three to four independent experiments. Dose response curves and log EC50 values shown in legends were generated with GraphPad Prism.

FIGS. 19A-19I show data demonstrating that β-blockers differentially engage 3-arrestin- and Gs-mediated β1AR signaling. FIGS. 19A-19E: Alprenolol- and carazolol-induced β1AR activation of ERK were diminished in β-arrestin-depleted cells, while the bucindolol response was unaltered. Wild type (WT) or β-arrestin1/2 knockout (β-arr1/2 KO) HEK293 cells transfected with β1ARs were stimulated with serial concentrations of indicated ligands for 5 min. The pERK/tERK ratio was normalized to the percentage of the maximum value in wide type group (FIGS. 19A-19C), or expressed as fold over unstimulated control (B). The level of β-arrestin dependency was calculated as loss of the fold increase at 10 μM ligand stimulation in β-arrestin1/2 knockout cells compared to that in wild type cells (FIG. 19C). FIGS. 19F-191: β-blockers induce variable level of Gs activation, and show no cooperativity with Cmpd-6. HEK293 cells transfected with β1ARs and GloSensor were pretreated with vehicle (DMSO) or 30 μM Cmpd-6 for 20 min, together with 100 nM β2AR-selective antagonist ICI118,551 to block the activation of endogenous β2ARs. Cells were then stimulated with serial doses of indicated ligands for 5 min. The cAMP level was monitored with GloSensor cAMP assay and normalized to the percentage of maximal isoproterenol-induced level in the same experiment. Data represent the mean±S.D. for five or six independent experiments as marked on the figure. Statistical comparisons were performed using two-way repeated (related)-measures ANOVA with Sidak correction for multiple comparisons (FIGS. 19A-19C, FIGS. 19F-19I), or two-tailed paired Student's t-test (FIG. 19D). *P<0.05, **P<0.01, ****P<0.0001.

FIG. 20 shows data demonstrating that differential doses of carvedilol decrease the level of ischemia/reperfusion-induced apoptosis in mouse hearts. Wild-type C57BL/6J mice were treated with vehicle or carvedilol (1, 5 or 20 mg/kg/day) for 3 days with Alzet osmotic pumps. The myocardium ischemia/reperfusion were produced by the ligation/release of LAD. Level of apoptosis was detected with TUNEL staining.

FIG. 21 shows a representative blot for the cardiomyocyte specific deletion of β-arrestin1/2. Lane 1-3: whole heart lysate from wild type, β-arrestin1 or β-arrestin2 global knockout mice. Lane 4-6: lysate of cardiomyocytes isolated from wild type, Arrb1^{flox/flox}/Arrb2^{flox/flox} or MyHC-Cre:Arrb^{flox/flox}/Arrb2^{flox/flox} mice.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment,” and the like refer to reducing the probability of developing a disease, disorder or condition in a subject, who does not have, but is at risk of or susceptible to developing a disease, disorder or condition.

The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject is a human subject suffering from a condition and/or disease in which the modulation of βarr activity is beneficial to the treatment of said condition and/or disease.

As used herein, the term “β-arrestin” or “βarr” refers to the ubiquitously expressed proteins that are involved in desensitizing G protein-coupled receptors (GPCRs), including all isoforms thereof (e.g., β-arrestin 1 (also referred to as βarr1), β-arrestin 2 (also referred to as βarr2), etc.).

As used herein, the term “p-blocker” or “p-adrenergic blocking agents” refers to those drugs that work by blocking the effects of the hormone epinephrine, also known as adrenaline. In practice, β-blockers cause the heart to beat more slowly and with less force. β-blockers are often found in three types: (i) β-1 (B1) receptors, which occur mainly in the heart and regulate cardiac activity; (ii) β-2 (B2) receptors, which occur in various organs and play a role in smooth muscle relaxation and metabolic activity; and (iii) β-3 (B3) receptors, which help break down fat cells. Suitable examples include, but are not limited to, acebutolol (Sectral™), atenolol (Tenormin™), betazolol (Kerlone^{M}), bisoprolol/hydrochlorothiazide (Ziac™), bisoprolol (Zebeta™), metoprolol (Lopressor™, Toprol XL™), nadolol (Corgard™), propranolol (Inderal™), sotalol (Betapace™), carvedilol (Coreg™), and combinations thereof. In one embodiment, the β-blocker comprises carvedilol (Coreg™).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like. In some embodiments, the disease comprises one that is treated, or can be treated and/or prevented, with a β-blocker. Such diseases include, but are not limited to, the following: (i) heart disease, including angina, congestive heart failure, hypertension (e.g., high blood pressure), irregular heartbeat, myocardial infarction (e.g., heart attack), rapid heartbeat (e.g., tachycardia), coronary heart disease, and the like; (ii) migraine; (iii) anxiety; (iv) overactive thyroid; (v) essential tremor; and the like.

The term “alkyl” as used herein, means a straight or branched chain saturated hydrocarbon. Representative examples of alkyl include, but are not limited to, methyl, ethyl, npropyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-oetyl, n-nonyl, and n-decyl.

The term “alkylene” or “alkylenyl,” or as used herein, means a divalent group derived from a straight or branched chain saturated hydrocarbon. Representative examples of alkylene/alkylenyl include, but are not limited to, —CH₂—, —CH₂CH₂—, —CH₂CH₂CH₂—, —CH₂CH(CH₃)CH₂—, and CH₂CH(CH₃)CH(CH₃)CH₂—.

The term “aryl,” as used herein, means phenyl or a bicyclic aryl. The bicyclic aryl is naphthyl, dihydronaphthalenyl, tetrahydronaphthalenyl, indanyl, or indenyl. The phenyl and bicyclic aryls are attached to the parent molecular moiety through any carbon atom contained within the phenyl or bicyclic aryl.

The term “halogen” means a chlorine, bromine, iodine, or fluorine atom.

The term “haloalkyl,” as used herein, means an alkyl, as defined herein, in which one, two, three, four, five, six, or seven hydrogen atoms are replaced by halogen. For example, representative examples of haloalkyl include, but are not limited to, 2-fluoroethyl, difluoromethyl, trifluoromethyl, 2,2,2-trifluoroethyl, 2,2,2-trifluoro-1, 1-dimethylethyl, and the like.

The term “cycloalkyl” as used herein, means a monocyclic all-carbon ring containing zero heteroatoms as ring atoms, and zero double bonds. Examples of cycloalkyls include, but are not limited to, cyclopropyl, cyclobutyl, cyclopentyl, cyclohexyl, cycloheptyl, and cyclooctyl. The cycloalkyl groups described herein can be appended to the parent molecular moiety through any substitutable carbon atom.

The terms “heterocycle” or “heterocyclic” refer generally to ring systems containing at least one heteroatom as a ring atom where the heteroatom is selected from oxygen, nitrogen, and sulfur. In some embodiments, a nitrogen or sulfur atom of the heterocycle is optionally substituted with oxo, Heterocycles may be a monocyclic heterocycle, a fused bicyclic heterocycle, or a spiro heterocycle. The monocyclic heterocycle is generally a 4, 5, 6, 7, or 8-membered non-aromatic ring containing at least one heteroatom selected from O, N, or S. The 4-membered ring contains one heteroatom and optionally one double bond. The 5-membered ring contains zero or one double bond and one, two or three heteroatoms. The 6, 7, or 8-membered ring contains zero, one, or two double bonds, and one, two, or three heteroatoms. Representative examples of monocyclic heterocycle include, but are not limited to, azetidmyl, azepanyl, diazepanyl, 1,3-dioxanyl, 1,4-dioxanyl, 1,3-dioxolanyl, 4,5-dihydroisoxazol-5-yl, 3,4-dihydropyranyl, 1,3-dithiolanyl, 1,3-dithianyl, imidazolmyl, imidazolidinyl, isothiazolinyl, isothiazolidmyl, isoxazolinyl, isoxazolidmyl, morpholinyl, oxadiazolinyl, oxadiazolidmyl, oxazolmyl, oxazolidinyl, oxetanyl, piperazmyl, piperidinyl, pyranyl, pyrazolmyl, pyrazolidinyl, pyrrolinyl, pyrrolidinyl, tetrahydrofuranyl, tetrahydropyranyl, tetrahydrothienyl, thiadiazolmyl, thiadiazolidinyl, thiazolinyl, thiazolidinyl, thiomorpholinyl, 1,1-dioxidothiomorpholinyl, thiopyranyl, and tritluanyl. The fused bicyclic heterocycle is a 7-12-membered ring system having a monocyclic heterocycle fused to a phenyl, to a saturated or partially saturated carbocyclic ring, or to another monocyclic heterocyclic ring, or to a monocyclic heteroaryl ring. Representative examples of fused bicyclic heterocycle include, but are not limited to, 1,3-benzodioxol-4-yl, 1,3-benzodithiolyl, 3-azabicyclo[3.1.0]hexanyl, hexahydro-¹H-furo[3,4-c]pyrrolyl, 2,3-dihydro-1,4-benzodioxinyl, 2,3-dihydro-1-benzofuranyl, 2,3-dihydro-1-benzothienyl, 2,3-dihydro-1H-indolyl, 5,6,7,8-tetrahydroimidazo[1,2-a]pyrazinyl, and 1,2,3,4-tetrahydroqumolinyl. Spiro heterocycle means a 4-, 5-, 6-, 7-, or 8-membered monocyclic heterocycle ring wherein two of the substituents on the same carbon atom form a second ring having 3, 4, 5, 6, 7, or 8 members. Examples of a spiro heterocycle include, but are not limited to, 1,4-dioxa-8-azaspiro[4.5]decanyl, 2-oxa-7-azaspiro[3.5]nonanyl, 2-oxa-6-azaspiro[3.3]heptanyl, and 8-azaspiro[4.5]decane.

The monocyclic heterocycle groups of the present disclosure may contain an alkylene bridge of 1, 2, or 3 carbon atoms, linking two nonadjacent atoms of the group. Examples of such a bridged heterocycle include, but are not limited to, 2,5 diazabicyclo[2.2.1]heptanyl, 2-azabicyclo[2.2.1]heptanyl, 2-azabicyclo[2.2.2]octanyl, and oxabicyclo[2.2.1]heptanyl. The monocyclic, fused bicyclic, and spiro heterocycle groups are connected to the parent molecular moiety through any substitutable carbon atom or any substitutable nitrogen atom contained within the group.

The term “oxo” as used herein refers to an oxygen atom bonded to the parent molecular moiety. An oxo may he attached to a carbon atom or a sulfur atom by a double bond. Alternatively, an oxo may be attached to a nitrogen atom by a single bond, i.e., an N-oxide.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl ““C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The above substituents may be abbreviated herein. For example, the abbreviations Me, Et, Ph and Bn represent methyl, ethyl, phenyl and benzyl, respectively. A more comprehensive list of standard abbreviations used by organic chemists appears in a table entitled Standard List of Abbreviations of the Journal of Organic Chemistry. The abbreviations contained in said list are hereby incorporated by reference.

For compounds described herein, groups and substituents thereof may be selected m accordance with permitted valence of the atoms and the substituents, and such that the selections and substitutions result in a stable compound, e.g., a compound that does not spontaneously undergo transformation such as by rearrangement, cyclization, elimination, etc.

In accordance with a convention used in the art, the group:

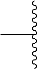

is used in structural formulae herein to depict the bond that is the point of attachment of the moiety or substituent to the core or backbone structure.

Unless otherwise defined, all technical terms used herein have the same meaning as commonly understood by one of ordinary skill in the art to which this disclosure belongs.

The present disclosure is based, in part, on the discovery by the inventors that that compound-6 positively augments carvedilol-stimulated cellular responses and elicits beta-adrenergic receptor-mediated cardioprotection in a mouse model of cardiac injury.

### A. Compositions

One aspect of the present disclosure provides compounds according to Formula (I) (e.g., herein termed Compound 6 or Cmpd 6, and A9 or analog A9):

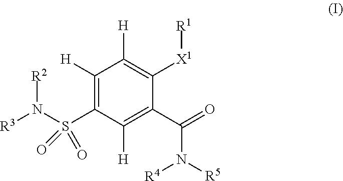

or a pharmaceutically acceptable salt, solvate, hydrate, prodrug, or derivative thereof, wherein:
R¹ is an aryl group optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆ alkyl)₂ —OC₃₋₆cycloalkyl, —NHC₃₋₆cycloalkyl, —N(C₁₋₆alkyl)(C₃₋₆cycloalkyl), and —N(C₃₋₆cycloalkyl)₂, and optionally the aryl is phenyl wherein two substituents join to form a 5- to 7-membered non-aromatic fused ring containing 1-2 heteroatom groups selected from NR^{1a} and O;
X₁ is O, N(H), N(C₁₋₄alkyl), S, S(O), S(O)₂, C(O), or CR^{1b}R^{1c};
R^{1a} is H or C₁₋₄alkyl;
R^{1b} and Ric are each independently hydrogen or C₁₋₄alkyl, or R^{1b} and Ric together with the carbon to which they are attached form a C₃₋₆cycloalkyl ring;
R² is hydrogen, C₁₋₆alkyl, or C₃₋₇cycloalkyl;
R³ is hydrogen, C₁₋₆alkyl, C₃₋₇cycloalkyl, or aryl, the aryl being optionally substituted with 1-5 substituents independently selected from the group consisting of C₁₋₆alkyl, C₁₋ₑhaloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂; alternatively, R² and R³ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R⁴ is hydrogen, C₁₋₆alkyl, or C₃₋₇cycloalkyl;
R⁵ is CHR^{5a}R^{5b}; alternatively, R⁴ and R⁵ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R^{5a} is aryl or —C₁₋₃alkylene-aryl, wherein each aryl in R^{5a} is optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆ alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂;
R^{5b} is X² or —C₁₋₃alkylene-X²; and
X² is —CN, —C(O)OH, —C(O)OC₁₋₄alkyl, —C(O)NH₂, —C(O)NHC₁₋₄alkyl, —C(O)N(C₁₋₄alkyl)₂, —SO₂NH₂, —SO₂NHC₁₋₄alkyl, or —SO₂N(C₁₋₄alkyl)₂.

In one embodiment, a compound of the invention is cmpd 6, having the formula

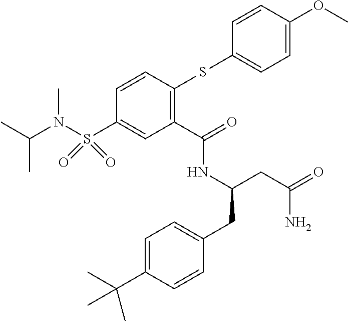 [SMILES: COc1ccc(Sc2ccc(S(=O)(=O)N(C)C(C)C)cc2C(=O)N[C@@H](CC(N)=O)Cc2ccc(C(C)(C)C)cc2)cc1]

and having the name (R)—N-(4-amino-1-(4-(tert-butyl)phenyl)-4-oxobutan-2-yl)-5-(N-isopropyl-N-methylsulfamoyl)-2-((4-methoxyphenyl)thio)benzamide.

In one embodiment, a compound of the invention is analog A9, having the formula

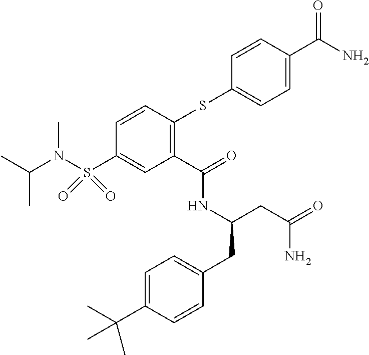 [SMILES: CC(C)N(C)S(=O)(=O)c1ccc(Sc2ccc(C(N)=O)cc2)c(C(=O)N[C@@H](CC(N)=O)Cc2ccc(C(C)(C)C)cc2)c1]

and having the name (R)—N-(4-amino-1-(4-(tert-butyl)phenyl)-4-oxobutan-2-yl)-2-((4-carbamoylphenyl)thio)-5-(N-isopropyl-N-methylsulfamoyl)benzamide.

### B. Pharmaceutical Compositions

In another aspect, the present disclosure provides compositions comprising one or more of compounds (e.g., Cmpd 6) as described herein and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies often treated with β-blockers, such as aspirin, nitroglycerin, NSAIDS, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making, levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### C. Methods of Use and Treatment

One aspect of the present disclosure provides a method of enhancing the effectiveness of a β-blocker being administered to a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the effectiveness of the β-blocker is enhanced.

Another aspect of the present disclosure provides a method of treating and/or preventing high blood pressure in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the high blood pressure is treated and/or prevented in the subject.

Another aspect of the present disclosure provides a method of treating and/or preventing heart disease in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the heart disease is treated and/or prevented in the subject.

Another aspect of the present disclosure provides a method of treating and/or preventing migraines in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the migraine is treated and/or prevented in the subject.

Another aspect of the present disclosure provides a method of treating and/or preventing glaucoma in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the glaucoma is treated and/or prevented in the subject.

Another aspect of the present disclosure provides a method of treating and/or preventing anxiety in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the anxiety is treated and/or prevented in the subject.

Another aspect of the present disclosure provides a method of treating and/or preventing overactive thyroid in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the overactive thyroid is treated and/or prevented in the subject.

Another aspect of the present disclosure provides a method of treating and/or preventing essential tremor in a subject, the method comprising, consisting of, or consisting essentially of administering to the subject a therapeutically effective amount of compound 6 (Cmpd 6), or a pharmaceutically acceptable salt, solvate, hydrate, prodrug, or derivative thereof, or a pharmaceutical composition thereof, and a beta-arrestin-biased β-blocker such that the essential tremor is treated and/or prevented in the subject.

In one embodiment, the beta-arrestin-biased β-blocker is carvedilol (Coreg™).

In one embodiment, the beta-arrestin-biased β-blocker is administered prior to the Cmpd 6. In another embodiment, the β-blocker is administered concurrently with the Cmpd 6. In yet another embodiment, the β-blocker is administered after the Cmpd 6.

Another aspect of the present disclosure provides a method of enhancing the effectiveness of a beta-arrestin-biased β-blocker being administered to a subject, the method comprising administering to the subject a therapeutically effective amount of a compound according to Formula (I):

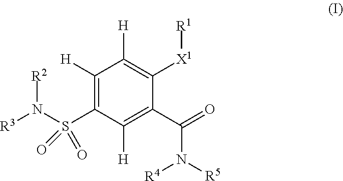

wherein:
R¹ is an aryl group optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆ alkyl)₂ —OC₃₋₆cycloalkyl, —NHC₃₋₆cycloalkyl, —N(C₁₋₆alkyl)(C₃₋₆cycloalkyl), and —N(C₃₋₆cycloalkyl)₂, and optionally the aryl is phenyl wherein two substituents join to form a 5- to 7-membered non-aromatic fused ring containing 1-2 heteroatom groups selected from NR^{1a} and O;
X₁ is O, N(H), N(C₁₋₄alkyl), S, S(O), S(O)₂, C(O), or CR^{1b}R^{1c};
R^{1a} is H or C₁₋₄alkyl;
R^{1b} and Ric are each independently hydrogen or C₁₋₄alkyl, or R^{1b} and Ric together with the carbon to which they are attached form a C₃₋₆cycloalkyl ring;
R² is hydrogen, C₁₋₆alkyl, or C₃₋₇cycloalkyl;
R³ is hydrogen, C₁₋₆alkyl, C₃₋₇cycloalkyl, or aryl, the aryl being optionally substituted with 1-5 substituents independently selected from the group consisting of C₁₋₆alkyl, C₁₋ₑhaloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂; alternatively, R² and R³ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R⁴ is hydrogen, C₁₋₆alkyl, or C₃₋₇cycloalkyl;
R⁵ is CHR^{5a}R^{5b}; alternatively, R⁴ and R⁵ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R^{5a} is aryl or —C₁₋₃alkylene-aryl, wherein each aryl in R^{5a} is optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆ alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂;
R^{5b} is X² or —C₁₋₃alkylene-X²; and
X² is —CN, —C(O)OH, —C(O)OC₁₋₄alkyl, —C(O)NH₂, —C(O)NHC₁₋₄alkyl, —C(O)N(C₁₋₄alkyl)₂, —SO₂NH₂, —SO₂NHC₁₋₄alkyl, or —SO₂N(C₁₋₄alkyl)₂. (Cmpd 6), or a pharmaceutically acceptable salt, solvate, hydrate, prodrug, or derivative thereof, or a pharmaceutical composition thereof, and
a β-arrestin-biased β-blocker such that the effectiveness of the β-arrestin-biased p-blocker is enhanced.

In one embodiment, the β-arrestin-biased beta blocker is administered to treat and/or prevent high blood pressure in the subject. In one embodiment, the β-arrestin-biased beta blocker is administered to treat and/or prevent heart disease in the subject.

In one embodiment, the β-arrestin-biased β-blocker is carvedilol (Coreg™).

In one embodiment, the β-arrestin-biased β-blocker is administered prior to the compound according to Formula (I). In one embodiment, the β-arrestin-biased β-blocker is administered concurrently with the compound according to Formula (I). In one embodiment, the β-arrestin-biased β-blocker is administered after the compound according to Formula (I).

In one embodiment, the compound according to Formula (I) is Cmpd 6, having the formula:

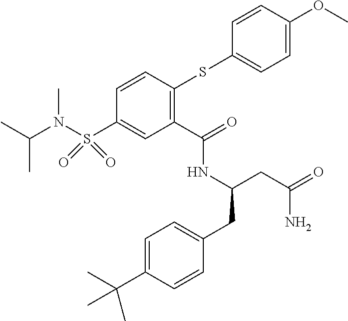 [SMILES: COc1ccc(Sc2ccc(S(=O)(=O)N(C)C(C)C)cc2C(=O)N[C@@H](CC(N)=O)Cc2ccc(C(C)(C)C)cc2)cc1]

or a pharmaceutically acceptable salt, solvate, hydrate, prodrug, or derivative thereof, or a pharmaceutical composition thereof.

In one embodiment, the compound according to Formula (I) is Cmpd A9, having the formula:

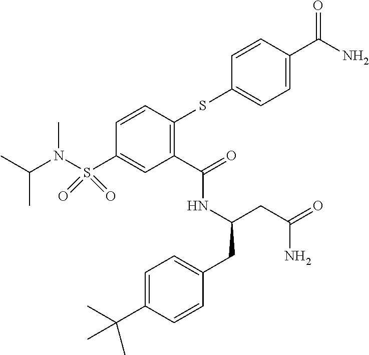 [SMILES: CC(C)N(C)S(=O)(=O)c1ccc(Sc2ccc(C(N)=O)cc2)c(C(=O)N[C@@H](CC(N)=O)Cc2ccc(C(C)(C)C)cc2)c1]

or a pharmaceutically acceptable salt, solvate, hydrate, prodrug, or derivative thereof, or a pharmaceutical composition thereof.

Another aspect of the present disclosure provides a method of treating and/or preventing high blood pressure or heart disease in a subject, the method comprising administering a therapeutically effective amount of a compound according to Formula (I), and a β-arrestin-biased β-blocker such that the high blood pressure or heart disease is treated and/or prevented in the subject.

In one embodiment, the compound according to Formula (I) is cmpd 6 or a pharmaceutically acceptable salt, solvate, hydrate, prodrug, or derivative thereof, or a pharmaceutical composition thereof. In one embodiment, the compound according to Formula (I) is analog A9 or a pharmaceutically acceptable salt, solvate, hydrate, prodrug, or derivative thereof, or a pharmaceutical composition thereof. In one embodiment, the beta-arrestin-biased beta blocker is carvedilol.

In one embodiment, the compound according to Formula (I) is administered after the beta-arrestin-biased beta blocker. In one embodiment, the compound according to Formula (I) is administered concurrently with the beta-arrestin-biased beta blocker. In one embodiment, the compound according to Formula (I) is administered prior to the beta-arrestin-biased beta blocker.

Another aspect of the present disclosure provides a pharmaceutical composition comprising a compound according to Formula (I) or a pharmaceutically acceptable salt, solvate, hydrate, prodrug, or derivative thereof, and a β-arrestin-biased β-blocker or a pharmaceutically acceptable salt, solvate, hydrate, prodrug, or derivative thereof.

In one embodiment, the compound according to Formula (I) is selected from the group consisting of Cmpd 6, Cmpd A9, combinations thereof, pharmaceutically acceptable salts, solvates, hydrates, prodrugs, and derivatives thereof. In one embodiment, the compound according to Formula (I) is Cmpd 6, or a pharmaceutically acceptable salt, solvate, hydrate, prodrug, or derivative thereof, or a pharmaceutical composition thereof. In one embodiment, the compound according to Formula (I) is Cmpd A9 or a pharmaceutically acceptable salt, solvate, hydrate, prodrug, or derivative thereof, or a pharmaceutical composition thereof. In one embodiment, the β-arrestin-biased β-blocker is carvedilol.

### D. Kits

The present disclosure further provides kits for modulating (e.g., increasing/augmenting) the effectiveness of a beta-arrestin-biased β-blocker in a subject and/or for preventing and/or treating a disease and/or condition as provided herein in a subject, the kit comprising, consisting of, or consisting essentially of a compound as provided herein (e.g., Cmpd 6) and a beta-arrestin-biased β-blocker (e.g., carvedilol).

In other embodiments, a kit may further include other components. Such components may be provided individually or in combinations, and may provide in any suitable container such as a vial, a bottle, or a tube. Examples of such components include, but are not limited to, (i) one or more additional reagents, such as one or more dilution buffers; one or more reconstitution solutions; one or more wash buffers; one or more storage buffers, one or more control reagents, pharmaceutical carriers and the like, (ii) one or more means of administering the compounds and additional therapeutics provided herein (e.g., syringe, dispensing cup, etc.); and the like. Kit components may also be provided individually or in combinations, and may be provided in any suitable container, such as a vial, a bottle, or a tube. In some embodiments, the kits disclosed herein comprise one or more reagents for use in the embodiments disclosed herein.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g., CD-ROM, diskette, flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

Yet another aspect of the present disclosure provides all that is disclosed and illustrated herein.

### EXAMPLES

The following examples are included as illustrative of the methods and compositions described herein. These examples are in no way intended to limit the scope of the invention. Other aspects of the invention will be apparent to those skilled in the art to which the invention pertains.

### Example 1: Positive Cooperativity Between with β-Arrestin-Biased β-Blocker Carvedilol and a Small Molecule Positive Allosteric Modulator of the β2-Adrenerigic Receptor

### A. Abstract

Among β-blockers that are clinically prescribed for heart failure, carvedilol is a first-choice agent with unique pharmacological properties. Carvedilol is distinct from other β-blockers in its ability to elicit β-arrestin-biased agonism which has been suggested to underlie its cardioprotective effects. Augmenting the pharmacologic properties of carvedilol thus holds the promise of developing more efficacious and/or biased β-blockers. The inventors recently identified compound-6 (cmpd-6), the first small molecule positive allosteric modulator (PAM) of the β2AR. Cmpd-6 is positively cooperative with orthosteric agonists at the β2AR and enhances agonist-mediated transducer (G-protein and β-arrestin) signaling in an unbiased manner. Here, it is reported that cmpd-6, quite unexpectedly, displays strong positive cooperativity only with carvedilol amongst a panel of structurally diverse β-blockers. Cmpd-6 enhances the binding affinity of carvedilol for the β2AR and augments its ability to competitively antagonize agonist-induced cAMP generation. Cmpd-6 potentiates β-arrestin1, but not Gs-protein, mediated high-affinity binding of carvedilol at the β2AR and β-arrestin-mediated cellular functions in response to carvedilol including ERK phosphorylation, receptor endocytosis and trafficking into lysosomes. Importantly, an analog of cmpd-6 which selectively retains positive cooperativity with carvedilol acts as a negative modulator of agonist-stimulated β2AR signaling. These cooperative properties of carvedilol and cmpd-6 have implications for fundamental understanding of GPCR allosteric modulation, as well as for the development of more effective biased beta blockers and other GPCR therapeutics.

### B. Materials and Methods

i. Reagents

All reagents used in this study were of molecular biology grade. All chemicals and ligands were obtained from Sigma-Aldrich (St. Louis, MO), unless mentioned otherwise. Compound-6 (Cmpd-6) and its analogs (A1-A12) were synthesized in-house as reported in U.S. patent application Ser. No. 16/269,877, fully incorporated herein by reference. Nanobody-6B9, Nb6B9, heterotrimeric Gs-apy and a minimal cysteine β-arrestin-1 truncated at residue 393, were purified following methods described earlier.

ii. Cell Culture

HEK293 and U2OS cells (ATCC, VA) were cultured in standard tissue culture incubator maintained at 37° C. and 5% CO₂ under humidified condition. HEK293 and U2OS cells were cultured in minimum Eagle's media (MEM) or Dulbecco's modified eagle media (DMEM) respectively, supplemented with 10% fetal bovine serum and 1× penicillin/streptomycin mix. HEK293 cell lines stably expressing the GloSensor (Promega; Madison, WI) cAMP reporter or the β2AR were maintained as described before. The HEK293 cell line used for total receptor endocytosis was obtained from Eurofins and maintained according to the manufacturer's recommendation. Clonal HEK293 cells stably expressing the Flag-β2AR or Flag-β2AR-YFP and U2OS cells stably expressing β2V2R—a chimeric β2AR with c-terminal tail of the Vasopressin receptor (V2R), were generated and maintained under G418 selection. Expi293F suspension cells were cultured as per manufacturers' instructions (Invitrogen, MA) in shaker incubator maintained at 37° C. and 8% CO₂ under humidified condition.

iii. Receptor Purification and HDL Reconstitutions

Full-length, N-terminal FLAG-tagged wild-type human β2AR was expressed in Sf9 insect cells using recombinant baculovirus and purified by n-Dodecyl-o-D-Maltopyranoside (DDM, Anatrace, Inc., OH) solubilization using anti-FLAG-M1 and alprenolol-ligand affinity chromatography followed by size-exclusion chromatography (SEC) as previously described (Kobilka B K, Anal Biochem 231(1): 269-271(1995)). The β2AR with the sortase consensus site (LPETGHH) inserted after amino acid 365 was expressed in Expi293F suspension cells (Invitrogen, MA) and purified using anti-FLAG-M1 affinity chromatography. Enzymatic (sortase) ligation of a synthetic phosphopeptide (V2Rpp) was done following methods established in previous study (Staus et al., Proc Natl Acad Sci USA 115(15): 3834-3839 (2018)) to generate β2AR-pP. Wild-type human β1AR, with an N-terminal FLAG-tag, was expressed in Expi293F cells by transient transfections using Expifectamine following manufacturers' instructions (Invitrogen, MA) and purified using methods established for the β2AR. In brief, β1AR transfected cells were grown for 60 h in the presence of Alprenolol (2 μM), harvested and solubilized in lysis buffer containing 1% DDM and 0.05% cholesteryl hemisuccinate (CHS). Clarified lysates were passed through anti-FLAG-M1 affinity column, washed, and eluted in cold elution buffer (20 mM HEPES, pH7.4, 100 mMNaCl, 0.1% DDM, 0.01% CHS, 5 mM EDTA, 2 μM Alprenolol, and 0.2 mg/ml Flag peptide). Affinity-purified β1AR was further cleaned-up by SEC and the monomeric receptor peak was pooled, concentrated and aliquots (with 20% glycerol) were snap frozen in liquid N2 and store at −80° C. until use. Detergent-free high-density lipoprotein (HDL) particle, also referred to as nanodiscs, reconstitution of respective Q-ARs (β1AR, β2AR and β2AR-pP) were carried out following previously established procedures (Ahn S, et al., Mol Pharmacol 94(2): 850-861 (2018); Staus D P, et al., Nature 535(7612): 448-452 (2016)). Receptor containing HDLs were isolated from receptor free HDL particles using anti-FLAG-M1 affinity chromatography followed by a further clean-up step by SEC.

iv. Membrane Purification

Membrane preparations from receptor expressing cells were carried out following previously reported methods (Strachan R T, et al., J Biol Chem 289(20): 14211-14224 (2014)), with minor modifications. Cells expressing β2AR-YFP or β2V2R were harvested in cold homogenization buffer (20 mM Tris-HCl, pH 7.4, 5 mM EDTA, 125 mM Sucrose, containing 0.2 mM PMSF and EDTA-free protease inhibitor cocktail). Cell suspensions were dounce-homogenized and subjected to differential centrifugation to obtain microsomal crude membrane fractions. Isolated membranes were triturated, using a syringe with 27G needle, in cold membrane resuspension buffer (50 mM Tris-HCl, pH 7.4, 150 mM NaCl, 12.5 mM MgCl₂, 2 mM EDTA, containing 10% glycerol and EDTA-free protease inhibitor cocktail). Aliquots of homogeneously resuspended membranes were snap frozen in liquid N₂ and stored at −80° C. until use.

v. Radioligand Binding

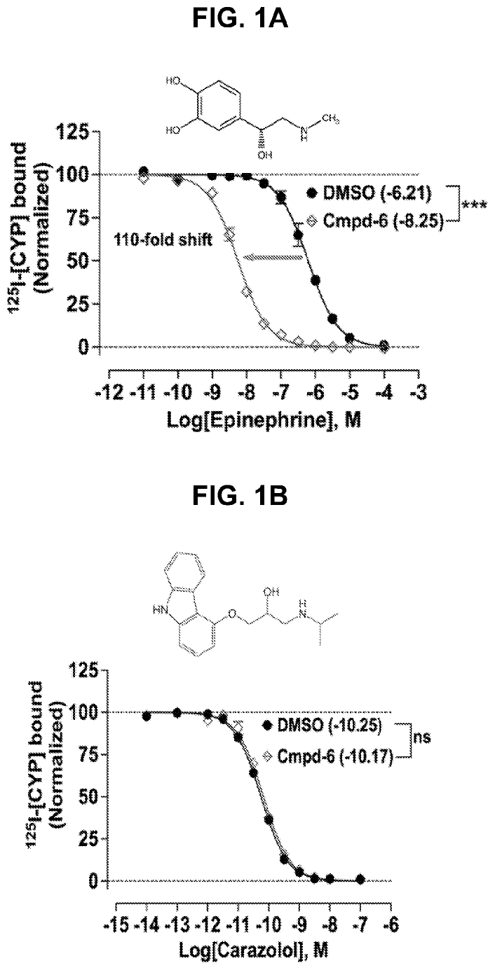

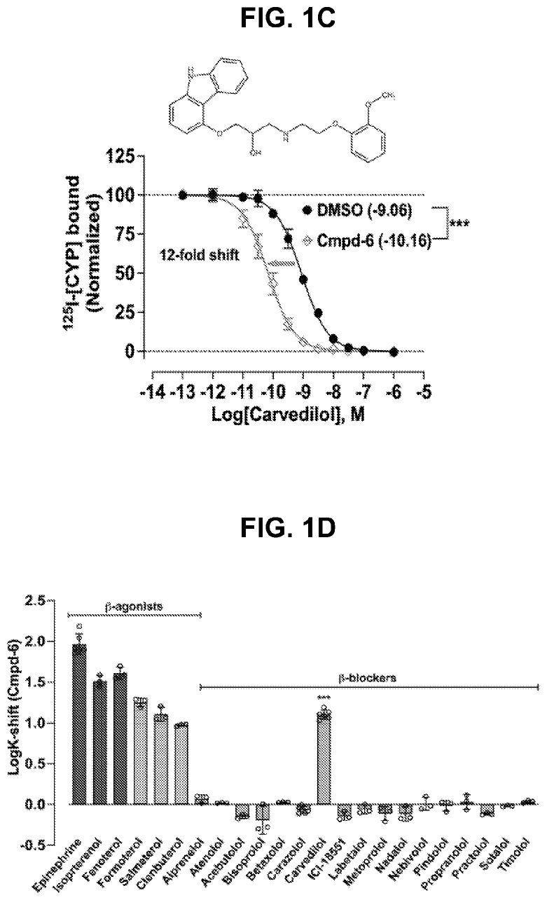

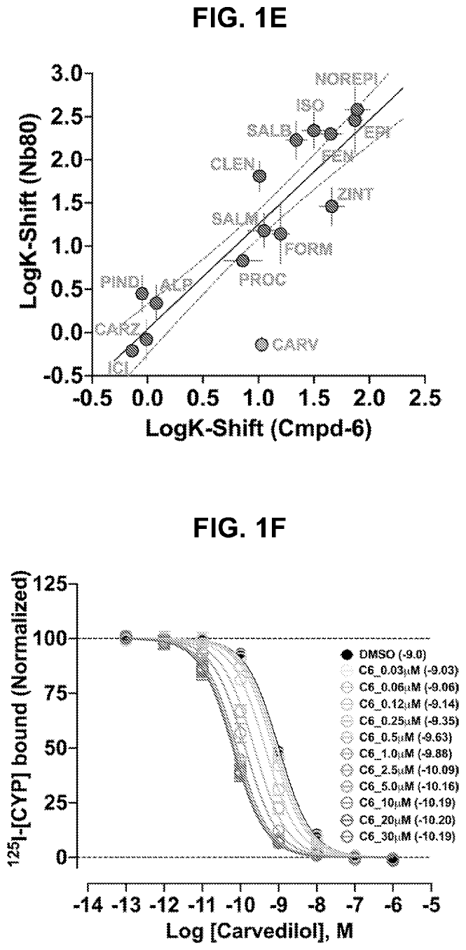

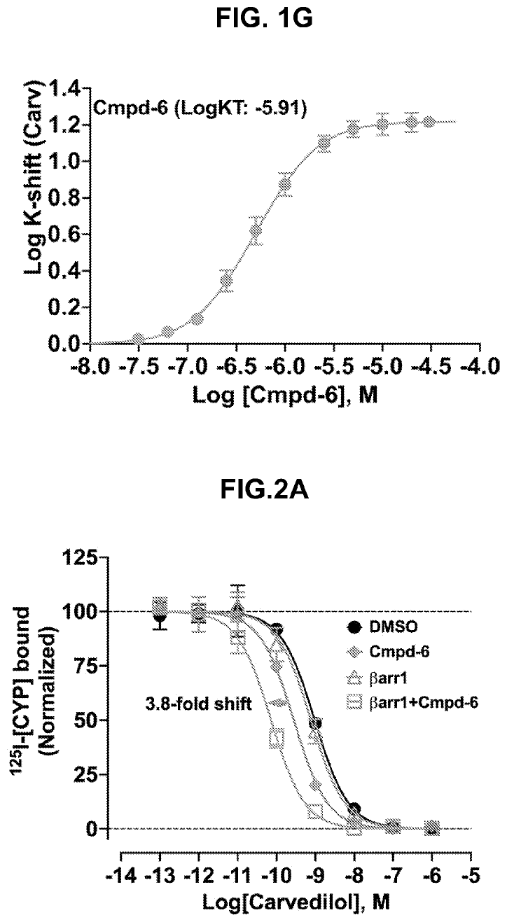

Competition radioligand binding assays were performed at respective 3-ARs (β1AR, β2AR and β2AR-pP) reconstituted into HDL particles (or nanodiscs) or from isolated membrane preparations (β2AR-YFP or β2V2R), in assay buffer composed of 20 mM HEPES, pH 7.4, 100 mM NaCl, 0.2 mg/mL BSA and 0.18 mg/mL ascorbic acid. [¹²⁵I]-Cyanopindolol (¹²⁵I-CYP, 2,200 Ci/mmol; PerkinElmer, MA) was used at 60 μM and was competed with a serially diluted dose of unlabeled ligands without or with cmpd-6 or its analogs (20 μM). Competition bindings at β2AR-pP were carried out in the absence of presence of cmpd-6 (1 μM) along with either Gs-apy heterotrimer (GsHet at 5 nM) or β-arrestin1 (Parrl at 250 nM). All binding assays were carried out till equilibrium at room temperature in a final reaction volume of 200 μl. Equilibrated binding reactions were harvested onto glass-fiber filters (GF/B), pre-soaked with 0.3% (vol/vol) polyethyleneimine in de-ionized water, using a 96-well Brandel harvester (Brandel, MD). The filters were rapidly washed with 10 ml cold wash buffer (20 mM HEPES, pH7.4, 100 mM NaCl), and the bound ¹²⁵I-CYP was measured using a 2470 Wizard2™ 2-Detector Gamma Counter (Perkin Elmer, MA). Competition binding data were analyzed in GraphPad Prism 9.0 using a non-linear regression curve fit and the one-site-Fit Log IC₅₀ equation, to derive the estimates of equilibrium binding constant (Kd) for respective conditions, and normalized counts per minute (cpm) values were plotted as mean±SD. The titration curves representing the change of carvedilol binding affinity with increasing concentrations of positive allosteric modulator (PAM) (data shown in FIGS. 1F, 5E, 6), were fitted using the following equation:

$$KT + β [ PAM ]$$

$$K - shift = \frac{{K}_{T} + β [ PAM ]}{{K}_{T} + β [ PAM ]}$$

Where, K-shift indicates the ratio of carvedilol dissociation constants measured in the absence and presence of allosteric modulator, KT is the dissociation constant of PAM for receptor binding, and β gauges the cooperativity factor between carvedilol and PAM binding. To estimate KT and β, the data were analyzed by non-linear regression with a user-defined version of the above equation added to the model library of GraphPad Prism. [³H]-R/S-Carvedilol (³H-Carv, 80 Ci/mmol; American Radiolabeled Chemicals, Inc., MO) binding was performed by a scintillation proximity assay (SPA) using Flag-tagged yittrium silicate (YSi) SPA microbeads (Perkin Elmer, MA). Nanodisc reconstituted β2AR-pP was incubated, in the aforementioned assay buffer, for 90 min at room temperature with 3H-Carv (1 nM) in the absence of DMSO (0.2%) or cmpd-6 (20 μM), GsHet (100 nM) and βarr1 (1 μM). Non-specific bindings were assessed by using saturating concentration of cold Propranolol (20 μM). Receptor was captured on YSi beads via Flag-tag and bound radioligand was detected using a Wallac 1450 microBeta scintillation counter (Perkin Elmer, MA). Bound specific cpm was expressed as ligand binding in fmol and plotted as bar graphs in GraphPad Prism.

[³⁵S]-Guanosine 5′-(γ-thio) Triphosphate (35S-GTPγS, 1250 Ci/mmol; Perkin Elmer, MA) binding to GsHet was performed using SPA in assay buffer containing 20 mM HEPES, pH7.4, 100 mM NaCl, 10 mM MgCl2, 10 μM GDP, 0.2 mg/mL BSA and 0.18 mg/mL Ascorbic acid. Nanodisc reconstituted β2AR were incubated with GsHet (100 nM) in the absence (DMSO) or presence of saturating concentration of cmpd-6 (20 μM) or A9 (20 μM), Nb-6B9 (1 μM), and stimulated with epinephrine (10 μM) or ICI-118551 (10 μM). ³⁵S-GTPγS was used at 200 μM, and binding reactions were carried out for 60 min at room temperature. Basal ³⁵S-GTPγS binding to Gs protein was determined in absence of agonist stimulation and non-specific binding was determined by including non-radioactive GTPγS (20 μM). Following incubations, the β2AR-Gs complexes were captured on YSi beads bound radioligand was detected using a Wallac 1450 microBeta scintillation counter (Perkin Elmer, MA). Specific “S-GTPγS binding for respective conditions were normalized to no ligand stimulation, expressed as fold over basal and plotted as bar graphs in GraphPad Prism.

vi. Measurements of cAMP Generation

Cyclic AMP (cAMP) production was monitored at endogenous β2AR in HEK293 cells stably expressing the plasmid for GloSensor luciferase enzyme (Promega; Madison, WI), a chemiluminescence-based cAMP biosensor. Cells were plated in 96-well, white clear-bottom plates at a density of ˜80,000 cells per well, and on the following day, chemiluminescence signals generated by the GloSensor luciferase were read using a CLARIOstar microplate reader (BMG Labtech; Cary, NC) as previously described (Ahn S, et al., Mol Pharmacol 94(2): 850-861 (2018)). Prior to ligand stimulations, cells were treated with GloSensor reagent and incubated at 27 degrees for 1 hour to allow equilibration. Cells were then treated with either cmpd-6 (20 μM) or a vehicle control (dimethylsulfoxide, DMSO) diluted in Hanks balanced solution (HBSS) supplemented with 20 mM HEPES, 0.05% BSA and 3-isobutyl-1-methylxanthine (IBMX) at a final concentration of 250 M. For cAMP generation by ligands tested in agonist mode, a serial dilution of either epinephrine or carvedilol diluted in HBSS supplemented with 20 mM HEPES and 0.05% BSA was then added to cells and changes in luminescence were read at various time points ranging from 5 to 35 minutes after addition of orthosteric ligand. To assess blockade of agonist-stimulated cAMP generation, respective β-blockers (carvedilol or metoprolol) were tested in antagonist mode. A serial dilution of these β-blockers was first added to cells and incubated at 27 degrees for ˜10 minutes followed by epinephrine at a final concentration of 1 M diluted in HBSS supplemented with 20 mM HEPES and 0.05% BSA was added to cells. Changes in chemiluminescence were then read at various time points after stimulation. Concentration response curves were then generated by plotting normalized chemiluminescence values and were analyzed in GraphPad Prism using non-linear regression analysis and log (inhibitor) vs. response function.

vii. Cell Surface ELISA

U2OS cells stably expressing HA-tagged β2V2R—a chimeric β2AR with C-terminal tail of the Vasopressin receptor (V2R) and βarrestin2-GFP were used to assess the extent of receptor internalization in intact cells using cell surface receptor ELISA (enzyme linked immunosorbent assay). Cells were cultured for overnight in 48-well tissue culture plates, and subsequently serum starved for overnight in DMEM containing 0.1% IgG- and protease-free BSA (Jackson ImmunuResearch Laboratories, Inc., PA), 20 mM HEPES, pH 7.4 and 1× GlutaMax (Invitrogen, MA). Following serum starvation, cells were stimulated for 16 h with a serial dilution of respective orthosteric ligands (epinephrine, carvedilol, or ICI-118551) in the absence (DMSO) or presence of cmpd-6 (5 μM). Thereafter, stimulated cells were fixed in freshly prepared 3.6% paraformaldehyde in HBSS for 30 min on ice, quenched with Tris-HCl (250 mM) and 0.3% H₂O₂, washed and blocked with 3% non-fat dry milk in HBSS. Cell surface Q2V2R were labeled using hrp-conjugated anti-HA-tag antibody (Sigma, clone 3F10 used at 1:2,500 dilution) and developed using 150 μl ultraTMB substrate (Thermo Fisher Scientific, MA) and quenched with 150 μl acidified HBSS (0.2N H₂SO₄). A 100 μl aliquot of quenched reaction from each condition was transferred to a 96-well plate for colorimetric readings at 450 nm using a CLARIOstar microplate reader (BMG Labtech, NC). Primary assay plates were gently washed with de-ionized water and stained with 0.2% Janus Green B in HBSS to estimate total cells per well for data normalizations. Following staining, cells were gently washed with de-ionized water and accumulated Janus Green B stain was extracted in 300 μl acidified HBSS (0.5N HCl). A 100 μl aliquot of the extracted whole cell staining from each condition was then transferred to a 96-well plate and colorimetric readings were taken at 595 nm. Absorbance values for cell surface receptor (450 nm) for each condition were normalized with corresponding whole cell staining (595 nm) and analyzed in GraphPad Prism using non-linear regression analysis and log (ligand) vs. response function to derive estimates of EC₅₀ values for respective ligands in the absence (DMSO) or presence of cmpd-6.

viii. Measurement of β2AR Endocytosis

β2AR endocytosis was monitored in a high-throughput way using a chemiluminescence-based enzyme fragment complementation assay (Eurofins, USA) according to the manufacturer's recommendations, with minor modifications. HEK293 cells, stably the expressing Enzyme Acceptor-tagged β2AR and endosome-localized ProLink-tethered protein, were plated in 96-well white, clear-bottomed plates at a density of ˜80,000 cells per well. On the following day, cells were treated with dimethyl sulfoxide (DMSO) or Cmpd-6 at 10-30 μM for 10 min, and then stimulated with a serial dilution of carvedilol for 16 h to accumulate signals over the time. The extent of β2AR trafficking to endosomes was measured as chemiluminescence signals resulting from the complementation of 0-galactosidase fragments (Enzyme Acceptor and ProLink) at endosomes and was detected on a CLARIOstar plate reader (BMG Labtech, NC) using a Detection kit (Eurofins, USA).

ix. ERK Phosphorylation

HEK293 cells stably expressing the β2AR were plated on 6-well plates at a density to achieve ˜50-70% confluency prior to serum-starvation on the following day. Serum-free medium was prepared by supplementing MEM with 0.1% bovine serum albumin (BSA), 10 mM HEPES (pH 7.4) and 1× penicillin/streptomycin into standard minimum Eagle's growth media. Following an overnight serum-starvation, cells were pretreated with Cmpd-6 at 5 μM, stimulated with a serial dilution of carvedilol for 5 min, and solubilized by directly adding 2x SDS-sample buffer. After sonication with a microtip for 15 sec, equal amounts of cellular extracts were separated on 4-20% Tris-glycine polyacrylamide gels (Invitrogen, MA) and resolved proteins were transferred onto nitrocellulose membranes (Bio-Rad, CA) for immunoblotting. Detection of total and phosphorylated ERK1/2 on immunoblots were carried out with rabbit polyclonal anti-phospho-p44/42 MAPK (Cell Signaling, used at 1:2,000 dilution) and anti-MAPK 1/2 (Upstate Technology Inc., used at 1:8,000 dilution) antibodies. Chemiluminescence signals were developed using the SuperSignal West Pico reagent (Thermo Fisher Scientific, MA), visualized using a ChemiDoc imaging system (Bio-Rad, CA), and quantified by a densitometry software, Image Lab (Bio-Rad, CA) and analyzed using GraphPad Prism 9.0.

x. Confocal Imaging

HEK293 cells stably expressing β2AR-YFP were plated on poly-d-lysine coated glass bottom dishes (MatTek, MA). Following day, cells were serum starved for overnight in MEM medium supplemented with 20 mM HEPES (pH7.4) and 1 mg/mL BSA. Cells were loaded with Lysotracker red dye as per manufacturer's instructions (Invitrogen, MA) for 30 min followed by an additional 30 min of stimulation with carvedilol (10 nM) or ICI-118551 (10 nM) in the absence (DMSO) or presence of Cmpd-6 (5 μM). Following ligand stimulations, cells were fixed for 30 min at room temperature in a freshly prepared 3.6% paraformaldehyde solution in HBSS, washed and imaged in FluroBrite DMEM (Invitrogen, MA). Samples were imaged using a Ziess LSM 710 confocal microscope equipped with the Yokogawa CSU-X1 spinning disc system and an Evolve 512 EMCCD camera (Photometrics, AZ). β2AR-YFP (green) was illuminated using a 488 nm laser and stained lysosomes (red) were imaged using 561 nm (for Lysotracker Red) laser. Fluorescent images were captured using both 63× and 100× oil objectives. Captured images were deconvoluted using no neighbor deconvolution to improve signal-to-noise ratio for quantitative analysis. Images were analyzed in 3i's SlideBook 6 program using the colocalization analysis tool. Background pixels were eliminated using Costes' automatic thresholding and pixels with overlapping red and green intensity were counted as collocated pixels. Each experimental condition surveyed 4-7 independent images with each image containing 15-20 cells. Fraction of collocated pixels were determined for each image by dividing the collocated pixel count by the total number of green (β2AR-YFP) and red (lysosomes) pixels. The resultant fractions of collocated pixels were normalized to the number of cells within an image to yield the colocalization indices for respective treatments and plotted using GraphPad Prism.

xi. Data Analysis and Statistics

Data analysis and plotting was performed using GraphPad Prism 9.0 (GraphPad, CA) and Microsoft Excel. Statistical comparisons were made using unpaired t-test, one-way or two-way analysis of variance (ANOVA) with Bonferroni's multiple comparisons post hoc tests. Experimental values are expressed as means±SEM. Differences in the mean values were considered to be significant at P<0.05.

### C. Results

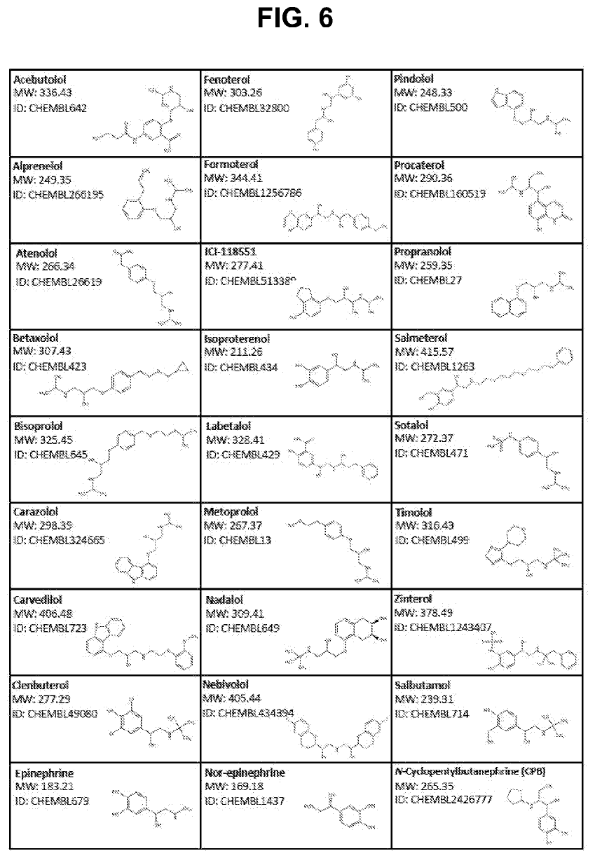

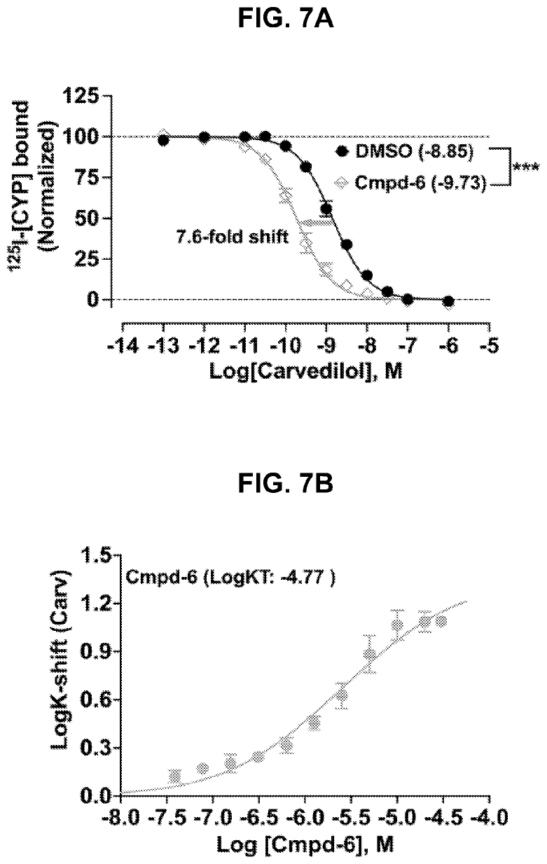

At the β2AR, cmpd-6 is cooperative with carvedilol amongst β-blockers: Cmpd-6 is a recently identified β2AR-specific positive allosteric modulator (PAM) which selectively shows positive cooperativity with P-agonists but not antagonists at the β2AR. Upon further examination with a structurally and pharmacologically diverse panel of p-AR ligands (FIG. 6), it was found that cmpd-6 is quite unexpectedly cooperative with the β-blocker carvedilol (FIGS. 1C and 1D). Consistent with the previous report, cmpd-6 shifts the 125I-CYP ([¹²⁵]Cyanopindolol) displacement binding curve of the full agonist epinephrine to the left by ˜2-log to higher affinity (FIG. 1C, EPI-DMSO Log IC50=−6.211, 95% CI [−6.245 to −6.177]; EPI+cmpd-6 Log IC₅₀=−8.254, 95% CI [−8.288 to −8.220]). As would be anticipated for an antagonist, there was no effect of cmpd-6 on the binding curve of carazolol, a close structural relative of carvedilol which shares the same carbazole head group (FIGS. 1C and 1D). However, surprisingly, the competition curve of the antagonist carvedilol was left-shifted by −1.2 log in presence of cmpd-6 (FIG. 1C, Carv-DMSO Log IC₅₀=−9.062, 95% CI [−9.093 to −9.031]; Carv+cmpd-6 Log IC50=−10.16, 95% CI [−10.20 to −10.13]). Amongst a total of 17 β-blockers tested (FIG. 1D) this phenomenon was observed only with carvedilol. Further, as shown in FIG. 1E, across a diverse set of P-ligands, there was a strong positive correlation (R2=0.83, without carvedilol) between cooperative effects of cmpd-6 with those of another PAM for the β2AR, nanobody-80 (Nb80) which pharmacologically behaves as a Gs mimic. The one exception from this correlation was carvedilol, which further underscores the positive cooperativity between cmpd-6 and carvedilol. The binding affinity of cmpd-6 for the β2AR in the presence of carvedilol was determined next in competition ligand binding assays (FIGS. 1F and 1G). Cmpd-6, in a dose-dependent manner, resulted in progressive left-shifts of ¹²⁵I-CYP displacement curves by carvedilol (FIG. 1F). By plotting the respective affinity K-shifts (difference in carvedilols' Log-IC₅₀ in DMOS vs [cmpd-6]) against concentration of cmpd-6. Titrations of cmpd-6 resulted in nested leftward curve shifts for carvedilol (FIG. 1F) from which it was determined that cmpd-6 binds the receptor with ˜1.2×10⁻⁶ M affinity in the presence of carvedilol (FIG. 1G, cmpd-6 Log K_{T}=−5.913, 95% CI [−5.969 to −5.852]). This binding affinity value of cmpd-6 is ˜4.3-fold stronger than its binding affinity for the agonist-occupied β2AR as previously determined by ITC (Ahn et al., 2018). Notably, while the PAM activity of cmpd-6 with respect to agonists is highly receptor subtype selective (β2AR>>β1AR), its cooperativity with carvedilol is also conserved at the β1AR (FIGS. 7A and 7B). The allosteric effect of cmpd-6 at the β1AR was saturable with a maximal curve shift of ˜0.9 log (FIG. 7A, DMSO Log IC50=−8.849, 95% CI [−8.885 to −8.813]; cmpd-6 Log IC₅₀=−9.731, 95% CI [−9.770 to −9.693]). The binding affinity of cmpd-6 was determined to be ˜1.7×10⁻⁵ M (FIG. 7B, cmpd-6 Log KT=−4.766, 95% CI [−5.146 to −3.951]). Taken together, these data identify a non-receptor subtype specific cooperativity between cmpd-6 and carvedilol amongst a large number of β-blockers. A detailed characterization of the allosteric effects of cmpd-6 on carvedilol-mediated β1AR signaling is reported, herein, and also in Wang et al., Mol Pharmacol., 100(6):568-579 (2021).

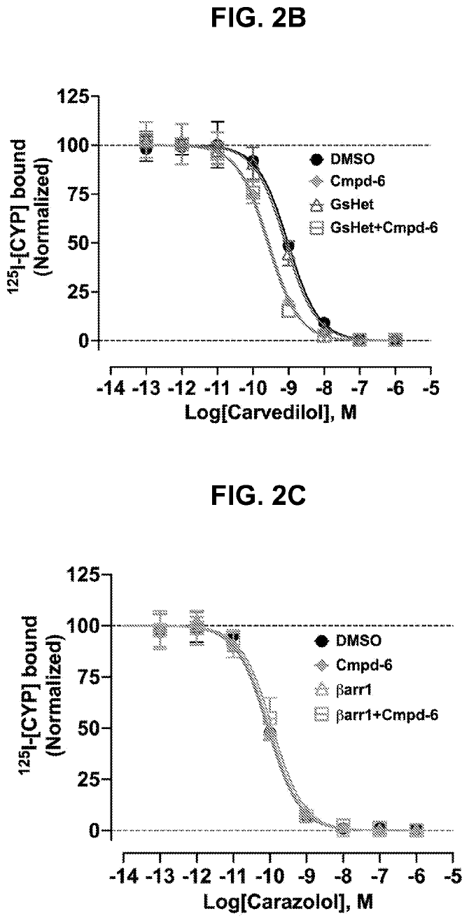

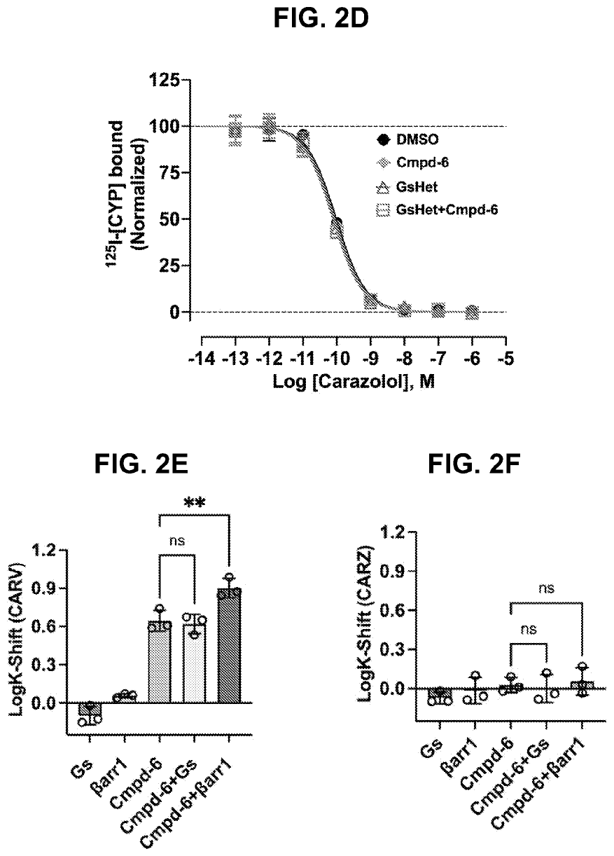

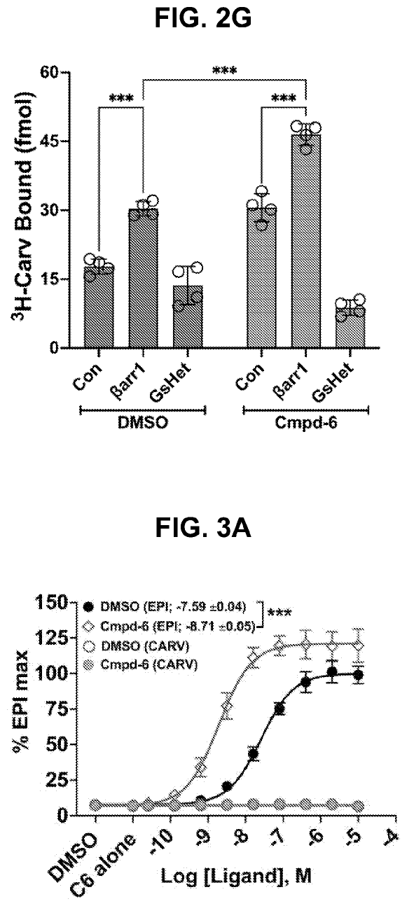

Cmpd-6 facilitates β-arrestin1-induced high affinity binding of carvedilol to the β2AR Carvedilol is a therapeutic β-blocker with a specific bias toward activating β-arrestin signaling. Thus, it was recognized that allosteric modulation of carvedilol function might be of therapeutic importance. The cooperative effects of cmpd-6 on transducer (Gs or β-arrestin1) coupling to the β2AR was assessed. Either carvedilol competition radioligand binding (FIGS. 2A-2F) or direct binding of radiolabeled carvedilol ([³H]-Carv) to the β2AR (FIG. 2G) in the absence (DMSO) or presence of cmpd-6 together with either transducer was monitored. To facilitate binding of βarr1 to the receptor, a synthetically phosphorylated C-tail of the vasopressin-2 receptor (V2R) was ligated in vitro at the C-terminal end of the β2AR (β2V2R-pP) using the sortase enzyme as previously described (Staus et al., 2018) and the phospho-peptide ligated receptor was reconstituted into detergent-free HDL particles. Cmpd-6 showed positive cooperativity with βarr1 (FIG. 2A, cmpd-6 Log IC₅₀=−9.554, 95% CI [−9.626 to ˜9.482]; βarr1+cmpd-6 Log IC₅₀=−10.14, 95% CI [−10.20 to −10.07]), but not with Gs (FIG. 2AB, GsHet+cmpd-6 Log IC50=−9.594, 95% CI [−9.714 to −9.475]) and enhanced the ability of carvedilol (but not carazolol, (FIGS. 2C and 2D) to compete against the radiolabeled tracer ²⁵I-CYP binding to the receptor. Remarkably, the combined application of cmpd-6 and βarr1 (but not Gs) resulted in a further high-affinity left-shift of the competition curve compared to that obtained using respective transducers tested individually (FIGS. 2E and 2F). Additionally, and consistent with the competition binding, it was shown that cmpd-6 in the presence of βarr1 (but not Gs) substantially increases the direct high-affinity binding of [³H]-Carv to the β2AR (FIG. 2G).

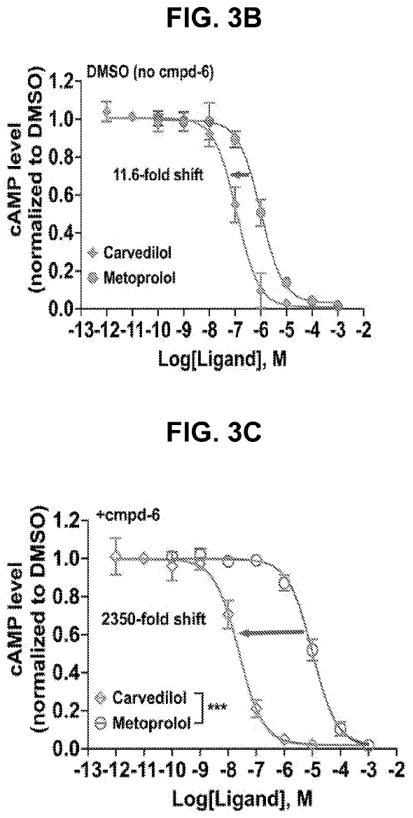

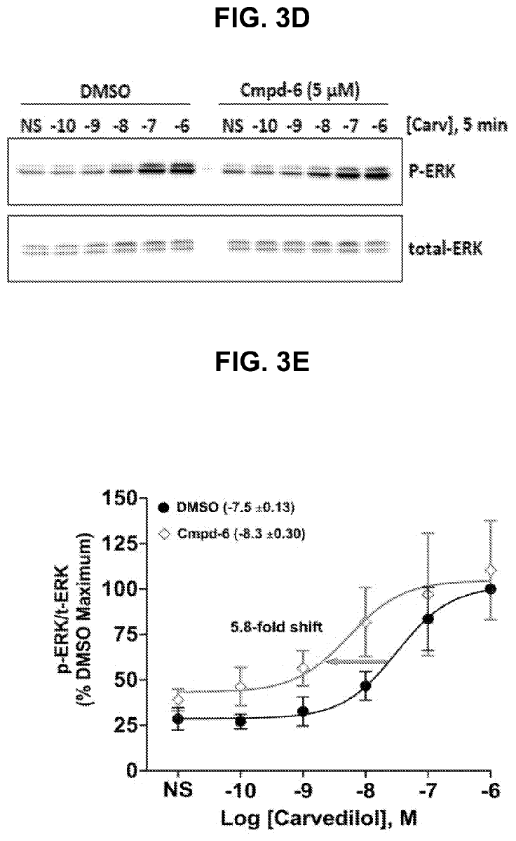

Cmpd-6 positively modulates carvedilol-stimulated cellular β2AR functions: To determine the cellular implications of the allosteric effect of cmpd-6 on carvedilol mediated signaling at the β2AR, a series of cell-based assays were performed. First, the activation of Gs was tested by monitoring cAMP generation. When used in an agonist mode, carvedilol alone and together with cmpd-6 did not stimulate any detectable levels of cAMP production, unlike the robust response to the agonist epinephrine stimulation, which was augmented by cmpd-6 (FIG. 3A, EPI Log EC₅₀=−7.587, 95% CI [−7.664 to −7.510]; EPI+cmpd-6 Log EC₅₀=−8.709, 95% CI [−8.807 to −8.611]). Next, carvedilol was tested in an antagonist mode, essentially evaluating its ability to block the agonist epinephrine-stimulated cAMP responses in comparison to a control ligand, metoprolol with which cmpd-6 had no positive cooperativity as shown in competition binding (FIG. 1D). Interestingly, in comparison to metoprolol, it was found that cmpd-6 substantially augments the blockade of agonist-stimulated cAMP generation by carvedilol (FIG. 3C, +cmpd-6, Carv Log Ki=−10.33, 95% CI [−10.44 to −10.22]; Meto Log Kᵢ=−7.703, 95% CI [−7.788 to −7.618]). Compared to metoprolol-mediated inhibition of Epi-stimulated cAMP generation, the presence of cmpd-6 remarkably led to −2350-fold leftward shift of the carvedilol dose-dependent inhibitory curve (FIG. 3C vs. ˜11-fold shift without cmpd-6, FIG. 3A). Further, this measure of fold-shift in inhibition of cAMP with carvedilol must be underestimated since cmpd-6 also potentiates the epinephrine-stimulated response. These results suggest that the allosteric modulation of carvedilol by cmpd-6 enhances the cellular β-blockade potency of the ligand. Then, the effect of cmpd-6 on carvedilol-stimulated ERK phosphorylation downstream of the β2AR was tested, which has been shown to be 0-arrestin-dependent. In HEK cells stably overexpressing the β2AR, cmpd-6 substantially enhanced (by ˜5.8-fold) the potency of carvedilol-simulated ERK phosphorylation (FIGS. 3D and 3E, DMSO Log EC₅₀=−7.504; 95% CI [−7.774 to −7.234]; +cmpd-6 Log EC₅₀=−8.268, 95% CI [−8.899 to −7.638]).

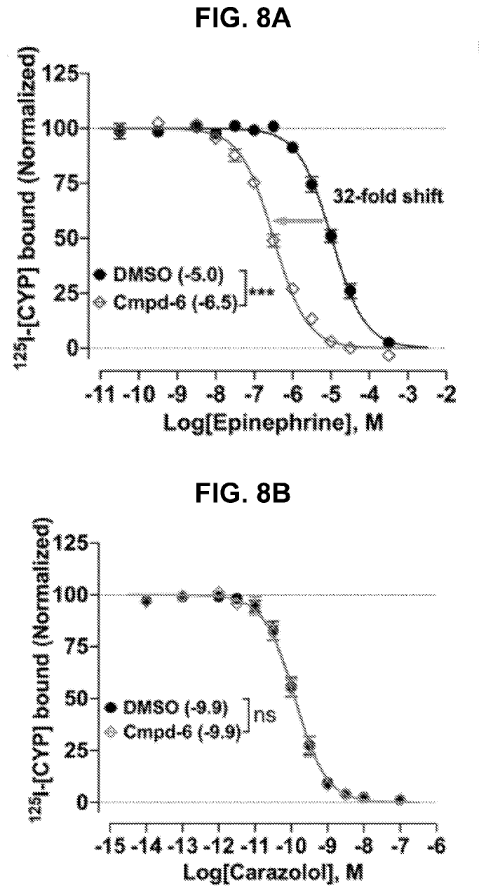

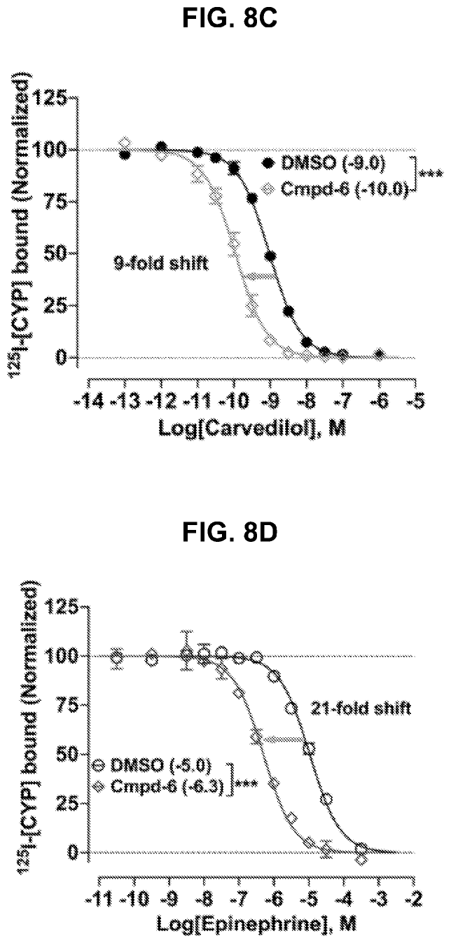

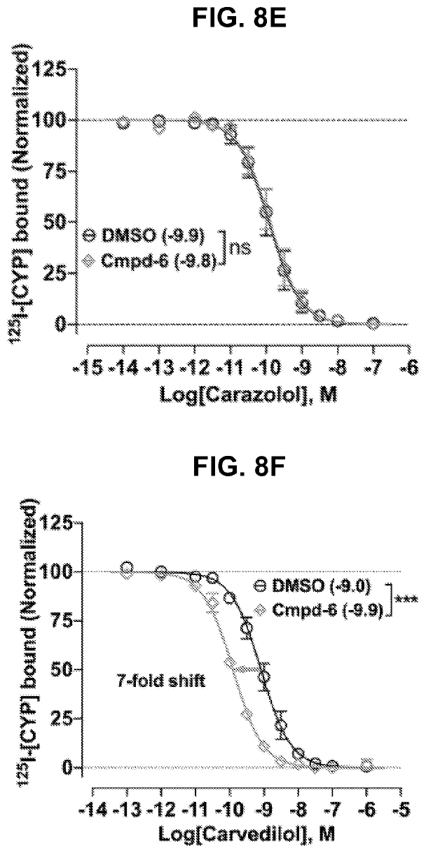

Cmpd-6 enhances carvedilol-stimulated internalization of the β2AR: To further evaluate the cellular effects of this positive cooperativity, the role of cmpd-6 on carvedilol-mediated receptor internalization, a function attributable to β-arrestins, was tested. To this end, U2OS and HEK293 cells stably expressing the chimeric β2V2R or the yellow fluorescent protein (YFP)-tagged β2AR, respectively, were employed. These recombinant versions of the human β2AR were pharmacologically validated by competition ligand binding using membrane preparations from respective cell lines (FIGS. 8A-8F). Consistent with in vitro binding data (FIG. 1C), both versions of the β2AR, in cell membrane preps, retained the cooperative effect of cmpd-6 with epinephrine and carvedilol but not with carazolol. The affinity-shifts for carvedilol elicited by cmpd-6 at the β2V2R (FIG. 8C, DMSO Log IC₅₀=−9.019, 95% CI [−9.043 to −8.996]; cmpd-6 Log IC₅₀=−9.959, 95% CI [−9.999 to −9.919]), and β2AR-YFP (FIG. 8F, DMSO Log IC₅₀=−9.086, 95% CI [−9.128 to −9.043]; cmpd-6 Log IC₅₀=−9.903, 95% CI [−9.936 to −9.870]) were conserved and comparable to the results obtained with the wild type β2AR (FIG. 1C).

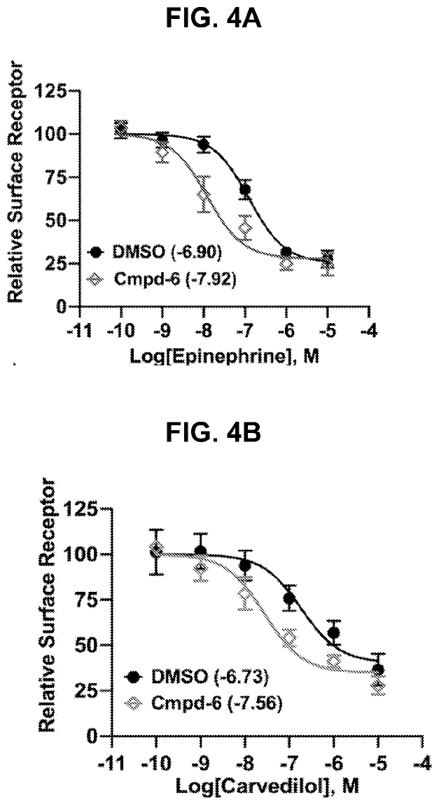

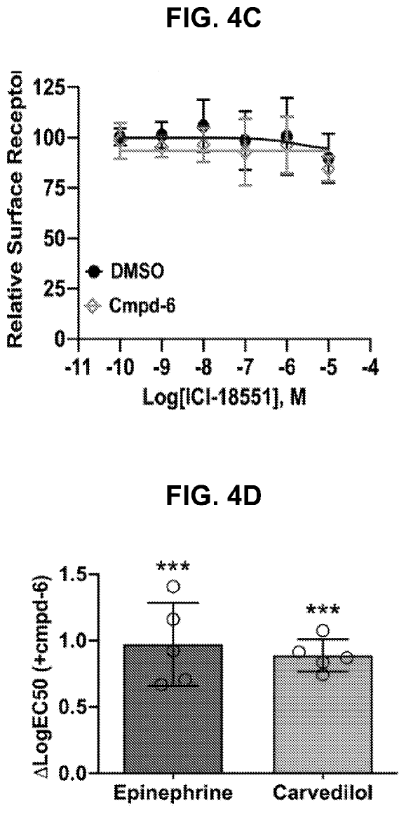

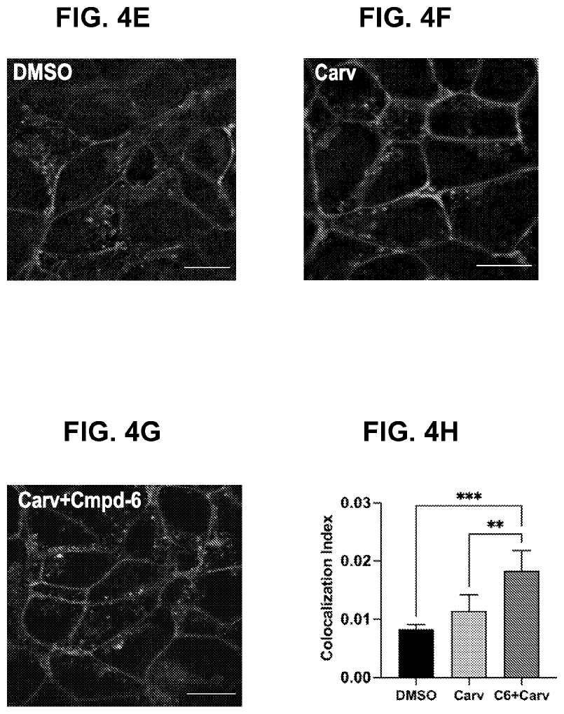

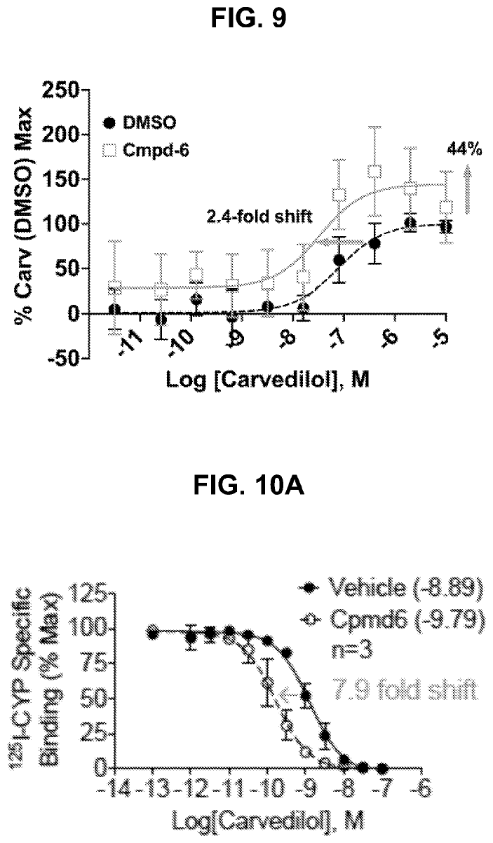

Multiple orthogonal assay formats were employed to evaluate carvedilol-stimulated receptor endocytosis in the absence (DMSO) or presence of cmpd-6: by measuring the loss of cell surface β2V2R by ELISA (FIG. 4A-D); by confocal imaging of β2AR-YFP trafficking to lysosomes (FIG. 4E-4H); and by DiscoveRx enzyme complementation assay for total receptor endocytosis (FIG. 9). Collectively, in all these aforementioned cellular assays, the effect of carvedilol together with cmpd-6 was significantly greater than that mediated by carvedilol alone. It was found that cmpd-6, as with the agonist epinephrine (FIG. 4A, DMSO Log EC₅₀=−6.901; 95% CI [“−7.008 to −6.795]; cmpd-6 Log EC₅₀=−7.916, 95% CI [−8.101 to −7.730]), enhanced the potency (by ˜6.8-fold change in EC50) of carvedilol stimulated endocytosis of the receptor as assessed by cell surface ELISA (FIG. 4B, DMSO Log EC₅₀=−6.731, 95% CI [“−7.018 to −6.445]; cmpd-6 Log EC₅₀=−7.561, 95% CI [−7.764 to −7.358]), as well as by total receptor endocytosis (FIG. 9). However, consistent with the binding data (FIG. 1D), no such cooperative effect of cmpd-6 on endocytosis of the receptor was observed in cells treated with the inverse agonist ICI-118551 (FIG. 4C). This further corroborates the positive cooperativity of cmpd-6 with the β-blocker carvedilol. Of note, even more effectively than agonists, carvedilol has been reported to target the β2AR to lysosomal compartments. Thus, the cooperative effect of cmpd-6 on the carvedilol-stimulated lysosomal targeting of the β2AR-YFP stably expressed in HEK-293 cells was tested. Following ligand stimulation, the colocalization of β2AR-YFP with the lysosomal marker dye (Lysotracker Red) was visualized and quantified (FIGS. 4E-4H). Interestingly, compared to DMSO control, cmpd-6 substantially augmented carvedilol-mediated lysosomal targeting of the β2AR. These data further underscore the signaling impact of the cooperative effects of cmpd-6 on carvedilol-mediated cellular functions. In essence, cmpd-6 not only enhances the cellular β-blockade potency of carvedilol, but also positively augments β-arrestin-mediated cellular signaling emanating from the carvedilol-occupied β2AR.

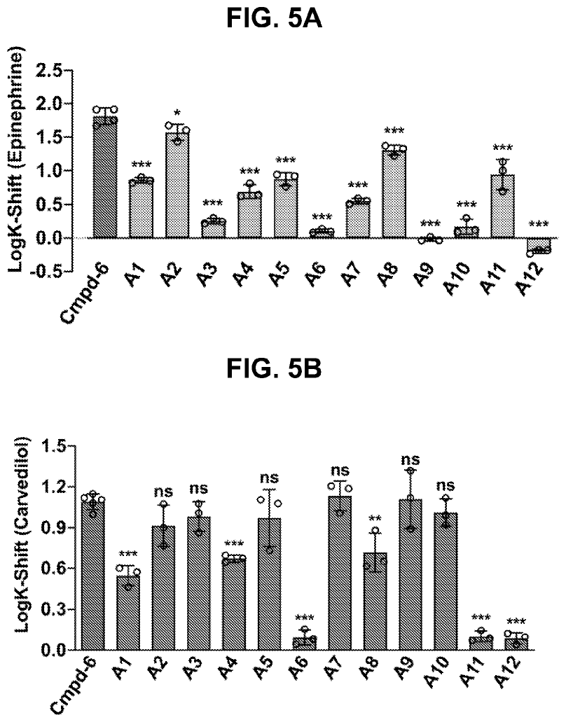

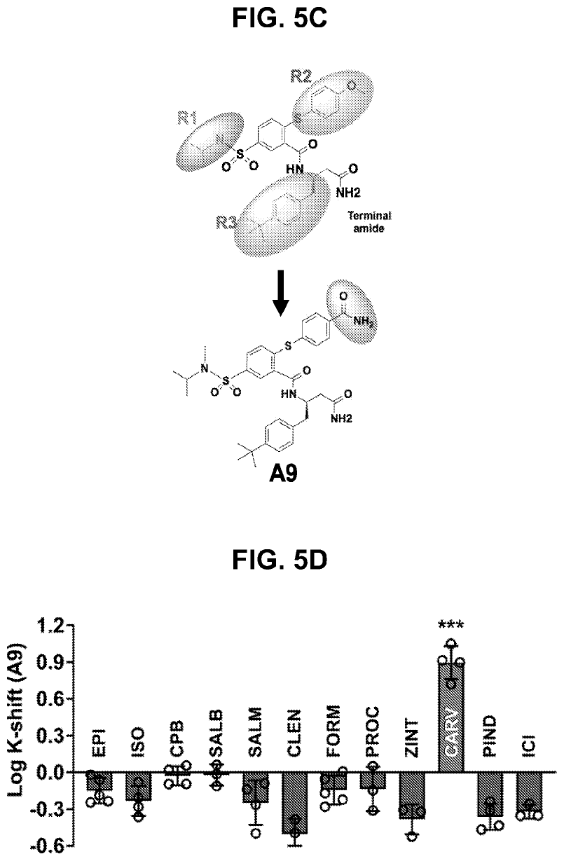

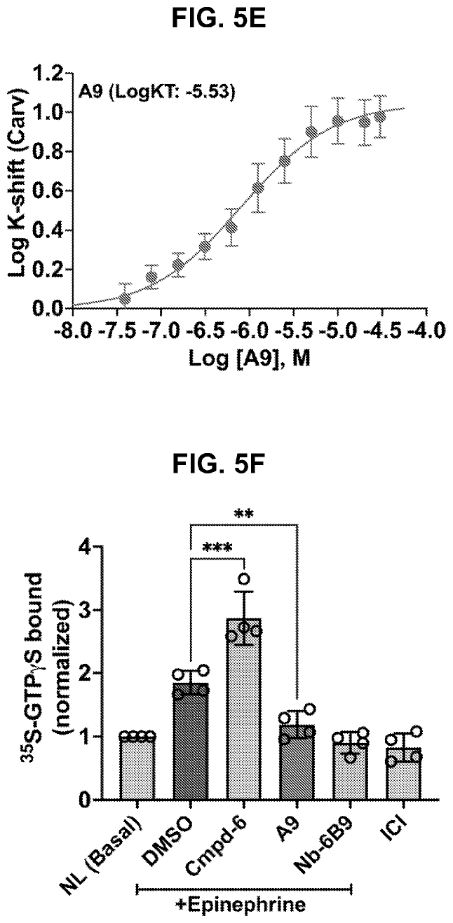

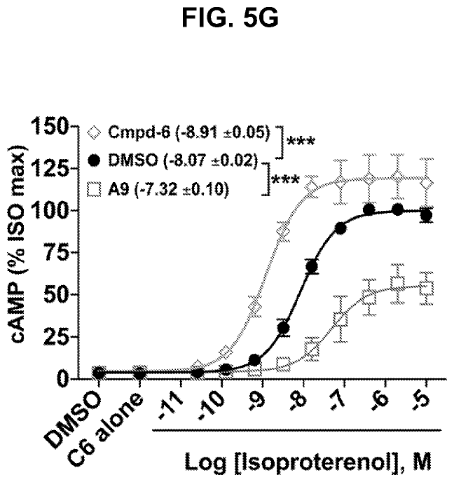

Development of a carvedilol-specific allosteric modulator: While cmpd-6 potentiates the β-arrestin-biased agonism of carvedilol, it is also a PAM which is positively cooperative with agonists at the β2AR in an unbiased manner. Accordingly, a set of chemically modified analogs of cmpd-6 (A1-A12) were tested, with the hopes to identify molecules which would retain the positive allosteric cooperativity with carvedilol while losing the PAM activity with β-agonists. Such modified analogs would not only be of potential therapeutic value but might also pave the way for the development of novel and biased allosteric drugs targeting other GPCRs. Amongst the several cmpd-6 analogs whose structures were previously reported (Liu X, et al., Science 364(6447):1283-1287 (2019)), the analog—A9 (FIGS. 5A and 5B) was identified to display the desired allosteric cooperative properties. Structurally, A9 differs from its parent cmpd-6 in carrying a terminal amide group at the R2 moiety (FIG. 5C). While A9 retains its positive cooperativity with carvedilol comparable to the level obtained with the parent cmpd-6, it shows absolutely no PAM activity with agonists including epinephrine (FIGS. 5A and 5D). The allosteric effect of A9 to affinity-shift carvedilol competition curves to the left is saturable and the analog has ˜2.9×10⁻⁶ M affinity for the carvedilol-bound β2AR (FIG. 5E, A9 Log K_{T}=−5.533, 95% CI [−5.737 to −5.247]), which is comparable to the ˜1.2×10⁻⁶ M affinity determined for cmpd-6 (FIG. 1G, cmpd-6 Log K_{T}=−5.913, 95% CI [−5.969 to −5.852]). Interestingly, unlike the PAM activity of cmpd-6 in potentiating P-agonist-stimulated responses, A9 remarkably shows a robust NAM activity for agonist-mediated β2AR functions. While cmpd-6 further augments the activation of heterotrimeric Gs by agonists, A9 markedly blocks the agonist-stimulated activation of Gs both in vitro and in cells. As shown by A9 mediated reduction in 35S-GTPγS binding to Gs in vitro (FIG. 5F). The binding of non-hydrolyzable 35S-GTPγS to Gs is driven by physical coupling of Gs to the β2AR and is increased in response to stimulation by the agonist epinephrine. However, in the presence of the antagonist/inverse agonist ICI-118551 no increase in 35S-GTPγS binding to Gs was observed. Additionally, and as expected, competition with Nb-6B9 (an affinity matured version of the Gs mimic nanobody, Nb80) which shares the transducer binding site also reduces 35S-GTPγS binding to Gs (FIG. 5F).

Furthermore, in cells, A9 (unlike cmpd-6) also functions as a classic NAM inhibiting agonist isoproterenol-stimulated cAMP generation downstream of the activated β2AR (FIG. 5G, DMSO Log EC₅₀=−8.073, 95% CI [−8.122 to −8.023]; cmpd-6 Log EC₅₀=−8.914, 95% CI [−9.021 to −8.807]; A9 Log EC₅₀=−7.320, 95% CI [−7.524 to −7.115]).

### D. Discussion

Herein, unexpected pharmacological cooperativity between the recently discovered PAM of the β2AR, cmpd-6 and the FDA approved β-blocker, carvedilol is shown. The findings herein unveil cmpd-6 as a positive allosteric modulator for the pharmacological activity of carvedilol at the β2AR as well as the closely related sub-type, β1AR. Remarkably, the cooperativity of cmpd-6 is highly specific to carvedilol amongst a diverse array of known p-blockers tested in this study. Using orthogonal experimental approaches, both in vitro and in cultured cells, it was demonstrated that cmpd-6 augments: the binding affinity of carvedilol for both 1- and β2AR, the potency of carvedilol's β-blockade activity at the β2AR; and carvedilol-stimulated β-arrestin mediated β2AR signaling functions such as ERK phosphorylation and receptor trafficking. Notably, a cmpd-6 analog, A9, was identified, which displays a complete switch in the allosteric properties from a PAM to a classic NAM and yet retains the distinctive positive cooperativity exclusively with carvedilol at the β2AR.

Ligands which bias GPCRs towards preferentially activating either G-protein or -arrestin mediated signaling hold immense therapeutic potential. Carvedilol is unique among β-blockers used in medicine in that it facilitates β-arrestin biased signaling (unlike other pβ-blockers) while still blocking the deleterious effects of chronic Gs mediated cAMP signaling downstream of activated P-adrenergic receptors. Indeed, findings from competition radioligand binding as well as direct binding of ³H-carvedilol indicate that cmpd-6 further potentiates the cooperativity between carvedilol and β-arrestin1, but not that with Gs. Previous studies have shown carvedilol stimulation resulting in ERK activation downstream of 01 and β2ARs, in a P-arrestin dependent manner. In the case of the β1AR this is through GPCR mediated transactivation of the epidermal growth factor receptor. This signaling is implicated to be cardioprotective by counteracting G-protein dependent catecholamine-induced toxicity and apoptotic pathways. Notably, clinical studies suggest that carvedilol may be superior to other β-blockers (such as metoprolol and propranolol) in preventing heart failure exacerbations and improving overall mortality in patients with reduced heart function. Carvedilol thus continues to be the drug of choice to treat patients with myocardial infarction and heart failure. The cardioprotective effects of carvedilol may be attributable to its unique ability to activate p-arrestin-mediated signaling pathways while potently blocking Gs activation. Interestingly, the findings herein show that cmpd-6 potentiates the ability of carvedilol, but not that of metoprolol, to block epinephrine-stimulated activation of Gs and cAMP generation. Additionally, cmpd-6 also augments the potency of carvedilol to stimulate β2AR-mediated ERK phosphorylation, which is known to be β-arrestin dependent and involved in cytoprotective signaling. These findings highlight the allosteric potential of cmpd-6 in positively augmenting the desirable signaling properties of carvedilol.

While carvedilol represents a prototypic β-arrestin-biased orthosteric drug at the 3-ARs, allosteric regulation of its varied signaling functions by cmpd-6 further expands the possibilities of developing improved β-blocker therapeutics even for biased orthosteric ligands. Indeed, by using a murine model of myocardial infarction, these findings quite remarkably demonstrate the clinical implications of the positive cooperativity between cmpd-6 and carvedilol.

In addition to desensitizing G-protein mediated signaling, β-arrestins are also known to play a pivotal role in receptor endocytosis. Over the past decade it has become evident that β-arrestins are recruited to membrane proteins (GPCRs, RTKs and even ion channels) where they interact with or serve as scaffolds for components of the cellular endocytic machinery (such as clathrin and AP2). β-arrestins thus function as key endocytic adaptors for activated GPCRs to facilitate their internalization resulting in either recycling or lysosomal degradation of the receptors. Uniquely, carvedilol (in contrast to other β-blockers) displays pharmacological properties that are akin to P-agonists with respect to β-arrestin mediated signaling. Carvedilol stimulation of cells results in GRK6-mediated phosphorylation of the β2AR as well as ubiquitination of the receptor by the E3 ligase, MARCH2. These signaling events have been reported to precede receptor internalization. In primary vascular smooth muscle cells, prolonged carvedilol treatment has been shown to trigger lysosomal trafficking and degradation of the β2AR. Consistent with these studies, it is shown herein that cmpd-6 potentiates carvedilol stimulated loss of cell surface β2AR leading to endocytosis and trafficking of the receptor to lysosomes. Although the effect of cmpd-6 on the above noted post translational modifications of the β2AR was not tested directly, the results herein on receptor endocytosis and lysosomal trafficking indicate that cmpd-6 enhances these carvedilol-stimulated responses.

While cmpd-6 was identified to be a highly selective PAM for the β2AR (Ahn et al., Mol Pharmacol 94(2): 850-861 (2018)), the current findings herein clearly indicate that the cooperativity between cmpd-6 and carvedilol is also preserved at the β1AR. This property of cmpd-6 deviates from the usual pattern of receptor sub-type selectivity which is a hallmark of allosteric modulators. Previous structural work suggests that the binding site of cmpd-6 in the agonist occupied β2AR is conserved in the β1AR, with different key residues mediating the receptor sub-type specific allosteric effect of cmpd-6. In the absence of any structural data on the exact binding site of cmpd-6 to either the carvedilol-bound β1AR or β2AR, it may be speculated that the site could topologically overlap with the binding site of cmpd-6 as that in the agonist-bound β2AR. While a given GPCR can have multiple, topologically distinct allosteric sites, it is also plausible that the binding site of cmpd-6 resides in a highly conserved structural motif that is common in this receptor family.

An emerging paradigm in GPCR structural biology is that GPCRs exist as ensembles of interconvertible inactive and active states, which are in conformational equilibrium. Compared to canonical β-blockers, carvedilol has been shown to elicit unique conformational changes in the β2AR. In particular, quantitative proteomic studies on labeling of solvent accessible reactive lysine and cysteine residues in the β2AR as well as findings from ¹⁹F-NMR studies on TM7 dynamics suggest a distinct conformational signature of the β2AR when bound to carvedilol. These results also accord with the unique β-arrestin biased agonism of carvedilol compared to other β-blockers, which presumably is displayed only by a minor fraction of the receptor population within the conformational spectrum of carvedilol-bound receptor. Based on the data presented herein, cmpd-6 appears to stabilize a carvedilol-bound distinct conformational signature of the β2AR.

Thus cmpd-6 is a well-suited allosteric tool for examining the conformational dynamics of carvedilol-bound β2AR. Interestingly, data herein shows that cmpd-6 has absolutely no cooperativity with the antagonist/inverse agonist, carazolol. While both ligands share an identical carbazole head-group, carvedilol differs from carazolol in having an extended aliphatic tail terminating with an anisole ring. From previously reported crystal structures of carvedilol- and carazolol-bound β2AR it is clear the carbazole moiety, which is shared by both ligands, occupies the orthosteric site situated deep in the transmembrane core (Bokoch M P, et al., Nature 463(7277): 108-112 (2010); Cherezov V, et al., Science 318(5854): 1258-1265 (2007); Ishchenko et al., IUCrJ 6(Pt 6):1106-1119 (2019)). However, a striking feature in the structure of carvedilol-bound β2AR is that the extended tail of carvedilol resides at a site distant from the deep orthosteric pocket. Based on this distinct binding modality of carvedilol, it may be hypothesized that the allosteric cooperativity between cmpd-6 and carvedilol is, in part, driven by the tail interactions of carvedilol with the β2AR. To discern the structural mechanism underlying this cooperativity, it will be of great interest to obtain atomic-level information on carvedilol-bound β2AR in complex with cmpd-6. Such structural studies will be useful not only for uncovering nuances of GPCR allostery but also for expanding current understanding of the dynamic nature of GPCR allosteric sites and biased agonism.

The ideal allosteric drug would cooperatively interact with orthosteric ligands to selectively potentiate signaling pathways of therapeutic importance. While cmpd-6 does augment the putative cardioprotective effects of carvedilol mediated by β-arrestin signaling, it also potentiates agonist-stimulated G-protein signaling at the β2AR. This agonist mediated signaling of cardiac β-receptors in part underlies the pathophysiology of heart failure. As such, from a potential therapeutic perspective, the dual cooperativity of cmpd-6 with P-agonists and carvedilol at the βeta2AR is diametrically opposed. Previous structure activity relationship (SAR) studies with cmpd-6 led to the synthesis of several chemically modified analogs of the parent compound. Herein, it is shown that the analog A9 has no cooperativity with agonists but still retains the positive allosteric cooperativity exclusively with carvedilol. A9 thereby serves as a small molecule prototype, which displays pharmacologic properties desirable of a potential allosteric therapeutic that together with carvedilol could be advantageous to abate cardiovascular ailments including heart failure.

In summary, these findings describe the ability of cmpd-6 and its analog A9 to allosterically potentiate the pharmacologic properties of carvedilol (a beta-arrestin-biased beta blocker), a key cardiovascular therapeutic. The discovery of this unexpected interaction has direct therapeutic implications and also serves to advance an understanding of GPCR allostery and biased agonism

### Example 2: β-Arrestin-Biased Allosteric Modulator Potentiates Carvedilol Stimulated βAdrenergic Receptor Cardioprotection

### A. Introduction

β1 adrenergic receptors (β1ARs) are central regulators of cardiac function and a drug target for cardiac disease. As a member of G protein-coupled receptor family, β1ARs activate cellular signaling by primarily coupling to Gs proteins to activate adenylyl cyclase and cAMP-dependent pathways, and the multifunctional adaptor-transducer protein β-arrestin. Carvedilol, a traditional β-blocker widely used in treating high blood pressure and heart failure by blocking βAR-mediated G-protein activation, can selectively stimulate Gs-independent β-arrestin signaling of βARs, a process known as β-arrestin-biased agonism. Recently a DNA-encoded small molecule library screen against agonist-occupied p2 adrenergic receptors (β2AR) identified Compound-6 (Cmpd-6) to be a positive allosteric modulator for agonists on β2ARs. Intriguingly, it was further discovered that Cmpd-6 is positively cooperative with the P-arrestin biased ligand carvedilol at β2ARs. Herein, the surprising finding is described that at β1ARs, unlike the case of β2ARs, Cmpd-6 is cooperative only with carvedilol and not agonists. Cmpd-6 increases the binding affinity of carvedilol for β1ARs and potentiates carvedilol-stimulated, β-arrestin-dependent β1AR signaling such as epidermal growth factor receptor transactivation and extracellular signal-regulated kinase activation, while having no effect on Gs-mediated cAMP generation. In vivo, Cmpd-6 enhances the anti-apoptotic cardioprotective effect of carvedilol in response to myocardial ischemia/reperfusion injury. This anti-apoptotic role of carvedilol is dependent on β-arrestins, since it is lost in mice with myocyte-specific deletion of β-arrestins. These findings demonstrate that Cmpd-6 is a selective β-arrestin-biased allosteric modulator of β1ARs and highlight its potential clinical utility in enhancing carvedilol-mediated cardioprotection against ischemic injury.

### B. Materials and Methods Reagents

Cmpd-6 was synthesized as previously described (see U.S. patent application Ser. No. 16/269,877, fully incorporated herein by reference). Carvedilol, isoproterenol, epinephrine, norepinephrine, metoprolol, carazolol, atenolol, bisoprolol, ICI-118,551 and EGF were purchased Sigma-Aldrich. Alprenolol was from Tocris Bioscience. Bucindolol was from Santa Cruz Biotechnology. Radiolabeled ¹²⁵I-cyanopindolol was from PerkinElmer Life Sciences.

β1AR expression and purification: The β1AR was expressed in and purified from Expi293F cells as previously described for the β2AR (Straus, et al, Proc Natl Acad Sci USA 115(15): 3834-3839 (2018)) with small modifications. FLAG-tagged β1AR was transfected into Expi293F cells (Invitrogen) with Expifectamine (Invitrogen) as described in the manufacturer's protocol. Cells were harvested 3 days after transfection and lysed by stirring in lysis buffer (10 mM Tris pH 7.4, 2 mM EDTA, 10 mM MgCl2, 5 units/mL benzonase, benzamidine, leupeptin, 1 μM alprenolol) for 30 min at room temperature. Cell membrane was pelleted by centrifugation at 32,000×g for 20 min at 4° C., homogenized in solubilization buffer (20 mM HEPES pH 7.4, 100 mM NaCl, 10 mM MgCl₂, 1% n-dodecyl-β-D-maltoside (DDM), 0.1% cholesterol hemisuccinate (CHS), 5 units/mL benzonase, benzamidine, leupeptin, 1 μM alprenolol), stirred at room temperature and 4° C. for 1 h each, and centrifuged at 32,000×g for 40 min at 4° C. The supernatant was supplemented with 2 mM CaCl₂ and loaded on M1-FLAG column at 2-3 ml/min at 4° C. The M1-FLAG column was washed with 5 column volumes each of low salt (100 mM NaCl) and high salt (500 mM NaCl) wash buffer (20 mM HEPES pH 7.4, 2 mM CaCl₂, 0.1% DDM, 0.01% CHS, benzamidine, leupeptin, 1 μM alprenolol). The wash cycle was repeated additional 3 times and then completed with 10 column volumes of low salt wash buffer. Receptor was eluted in elution buffer (20 mM HEPES pH 7.4, 100 mM NaCl, 0.1% DDM, 0.01% CHS, 0.2 mg/mL FLAG-peptide, 5 mM EDTA, 1 μM alprenolol) and cleaned-up by size exclusion chromatography with Superdex 200 Increase Column (GE Healthcare Life Sciences).

High-density lipoprotein (HDL) reconstitution: Reconstitution of purified β1ARs into HDL particles was performed as previously described (Ahn et al., 2018) for the β2AR. In brief, 2 μM purified β1AR was incubated with 100 μM apolipoprotein A1 (MSP1) and 8 mM phosphatidylcholine/phosphatidylglycerol (3:2 molar ratio mixture) in buffer (20 mM HEPES pH7.4, 100 mM NaCl and 100 mM cholate) for 1 h at 4° C. Biobeads (BioRad) were then added and rotated overnight at 4° C. to remove detergent. β1AR-HDL particle was isolated from empty HDL particles using MI-FLAG chromatography.

Competition binding assay: Radioligand competition binding assay was performed with the radiolabeled ¹²⁵I-cyanopindolol (CYP) (PerkinElmer) as previously described for the β2AR (Ahn et al., Mol Pharmacol 94(2): 850-861 (2018)). The β1AR-HDL particles were incubated with 60 μM ¹²⁵I-CYP, vehicle (DMSO) or 25 μM Cmpd-6, and a serial dilution of indicated orthosteric ligands in assay buffer (20 mM HEPES pH 7.4, 100 mM NaCl, 0.1% BSA, 1 mM ascorbic acid) at room temperature for 90 min. Non-specific binding was determined in the presence of 20 μM propranolol. The reaction was terminated and harvested by rapid filtration onto 0.3% polyethyleneimine-treated GF/B glass fiber filter paper (Brandel) using a harvester (Brandel). ¹²⁵I-CYP on the filter paper was measured by a WIZARD2 2-Detector Gamma Counter (PerkinElmer). Binding data were analyzed in GraphPad Prism using a one-site competition-binding log IC₅₀ curve fit with data points weighted equally. CPM values were normalized as percentage of the maximal ¹²⁵I-CYP binding level and plotted as mean S.D.

GloSensor assay: Analysis of cAMP accumulation was performed in HEK293 cells transiently transfected with β1AR and GloSensor constructs. The day before transfection, 5.5 million cells were seeded in one 10 cm tissue culture dish. The cells were transfected with 6 μg Glosensor plasmid DNA and 25 ng β1AR plasmid using FuGene6 (Promega) transfection reagent. After one day, 100,000 transfected cells were seeded to each well of 96-well plate in assay media (MEM with 2% FBS). The next day, the cells were incubated with the GloSensor reagent [Promega; 4% (v/v)] at room temperature for 2 h, pretreated with 100 nM ICI118,551 to block activation of endogenous β2AR, then stimulated with indicated ligands for 5 min. Luminescence was detected on a Biotek Neo2 microplate reader.

EGFR transactivation assay: The level of EGFR transactivation was measured by the endocytosis BRET assay as previously described (Namkung Y, et al., Nat Commun 7: 12178 (2016)), with slight modifications. HEK293 cells stably expressing β1ARs were transfected with 250 ng of EGFR-RlucII and 1 μg of rGFP-FYVE constructs. The day following transfection, cells were re-seeded onto poly-L-ornithine coated 96-well plates at ˜25,000 cells per well. The next day, cells were pretreated with vehicle (DMSO), 30 μM Cmpd-6 or 10 μM AG1478 in assay buffer (HBSS, 20 mM HEPES and pH 7.4) for 20 min at 37° C., and then stimulated with ligand at indicated concentration for 25 min. The cell-permeable substrate coelenterazine 400a (final concentrations of 5 μM) was added 3˜6 min before BRET measurements. The BRET measurements were performed with a Neo2 (BioTek) microplate reader with a filter set (center wavelength/band width) of 410/80 nm (donor) and 515/30 nm (acceptor).

ERK activation assay: HEK293 cells with stable overexpression of FLAG-tagged β1AR or β-arrestin knockout were described previously. Cells were periodically treated with BM Cyclin (Roche) to avoid mycoplasma contamination. Cells were incubated for 4 h in serum-free medium supplemented with 0.1% BSA, 10 mM HEPES and 1% penicillin-streptomycin, pretreated with vehicle (DMSO) or 30 μM Cmpd-6 for 20 min, and stimulated with indicated ligands for 5 min. Following stimulation, cells were lysed in lysis buffer (20 mM Tris, pH7.4, 137 mM NaCl, 20% glycerol, 1% Nonidet P-40, 2 mM sodium orthovanadate, 1 mM PMSF, 10 mM sodium fluoride, 10 μg/ml aprotinin, 5 μg/ml leupeptin and phosphatase inhibitors) by rotating for 30 min at 4° C. Cell lysate samples were separated by SDS-PAGE, transferred to PVDF membrane (Bio-Rad) and subjected to immunoblotting with anti-MAPK 1/2 (EMD Millipore) and anti-p44/42 MAPK (Cell Signaling) antibodies. Immunoblots were detected using enhanced chemiluminescence (Thermo Fisher Scientific) and analyzed with ImageJ software. The densitometry values from pERK blots were normalized to respective total ERK blots, and normalized to the maximum response of control group in each experiment.

Isoproterenol-induced hemodynamic in mouse heart: Eight to twelve-week-old C57BL/6J wild-type mice of both sexes were used. Animal experiments carried out were handled according to approved protocols and animal welfare regulations mandated by the Institutional Animal Care and Use Committee of Duke University Medical Center. Mice were treated with vehicle, carvedilol (1, 5 or 20 mg/kg/day), or combination of carvedilol (1 mg/kg/day) and Cmpd-6 (5 mg/kg/day) for 3 days with Alzet osmotic pump (Durect) implanted into mice subcutaneously. After treatment, mice were anesthetized with ketamine (100 mg/kg) and xylazine (2.5 mg/kg). After bilateral vagotomy, a 1.4 French (0.46 mm) high fidelity micromanometer catheter (ADInstruments) connected to a pressure transducer (ADInstruments) was inserted into the left ventricle to monitor blood pressure. Basal blood pressure was recorded at steady state after the catheter insertion (2-3 min after insertion). Graded doses of isoproterenol were administered at 45 sec intervals by intravenous injection through a jugular vein. The blood pressure was monitored continuously and recorded at the steady state (35-45 sec after each injection). Data analysis was performed using LabChart 8 software (ADInstruments).

Ischemia reperfusion-induced cell apoptosis in mouse heart: Eight to twelve-week-old C57BL/6J wild-type mice or eight to twenty-seven-week-old αMyHC-Cre:Arrblflox/flox/Arrb2flox/flox mice of both sexes were used. Animals were randomly assigned to, and the researchers were blinded for, treatment groups. Alzet osmotic pump (Durect) were implanted into mice subcutaneously to deliver vehicle (DMSO), Cmpd-6 (5 mg/kg/day), carvedilol (1 mg/kg/day or 20 mg/kg/day), or combination of Cmpd-6 (5 mg/kg/day) and carvedilol (1 mg/kg/day). After 3 days of treatment, cardiac ischemia was produced by the ligation of left anterior descending coronary artery. After 45 min, the ligation was released to allow the blood flow to be restored for 45 min. The mice hearts were perfusion-fixed with 4% paraformaldehyde, excised from the body, and placed in 30% sucrose in PBS for 2-4 h at 4° C. The hearts were then embedded in optimum cutting temperature compound (Sakura Finetek) and snap frozen in liquid nitrogen. TUNEL staining on the cryo-sections were performed with in situ cell death detection kit (Sigma-Aldrich) according to the manufacturer's protocol. Sections were then mounted in ProLon Diamond Antifade Mountant with DAPI (Thermo Fisher Scientific). Images were recorded with Zeiss Axio Observer Z1 confocal microscope with 20× objective. In each heart, sections from different groups (from base to apex of the tissue, apart by 0.5 mm) were stained, and the group with maximum TUNEL-positive cells were used for quantification. At lease 9 images from 3 sections were quantified for each heart. The number of TUNEL-positive cells and the total number of nuclei as determined by DAPI staining were counted using ImageJ software.

Statistical analysis: Data are expressed as mean±S.D. Statistical comparisons were performed using one-way ANOVA with Tukey correction or two-way repeated-measures ANOVA with Sidak correction for multiple comparison in GraphPad Prism. Differences were considered statistically significant at P<0.05.

### C. Results

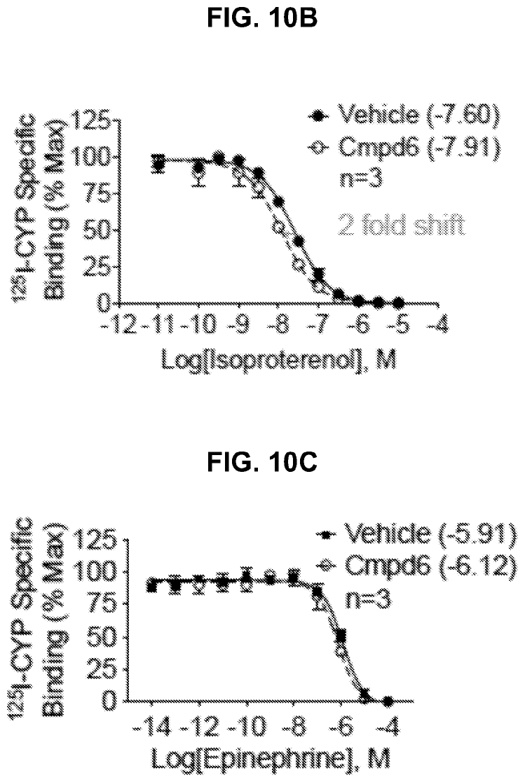

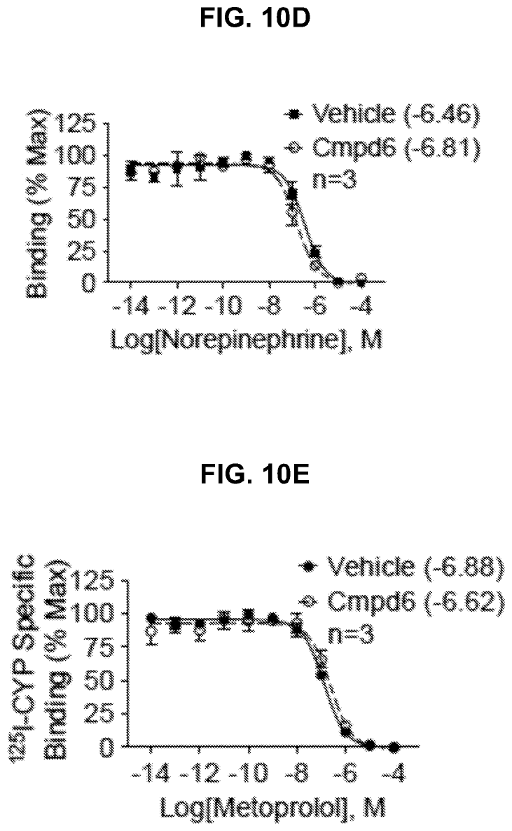

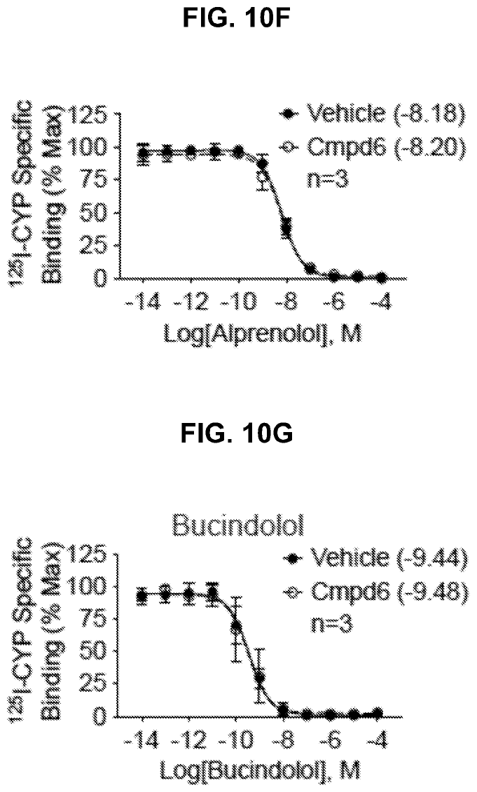

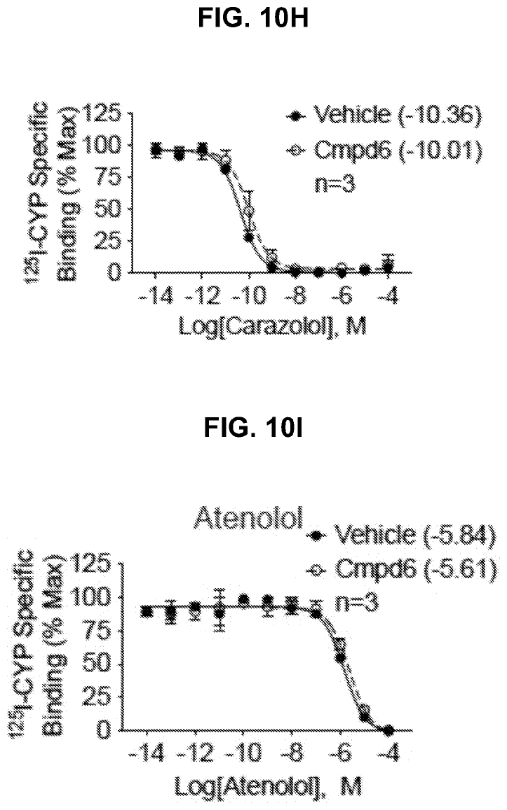

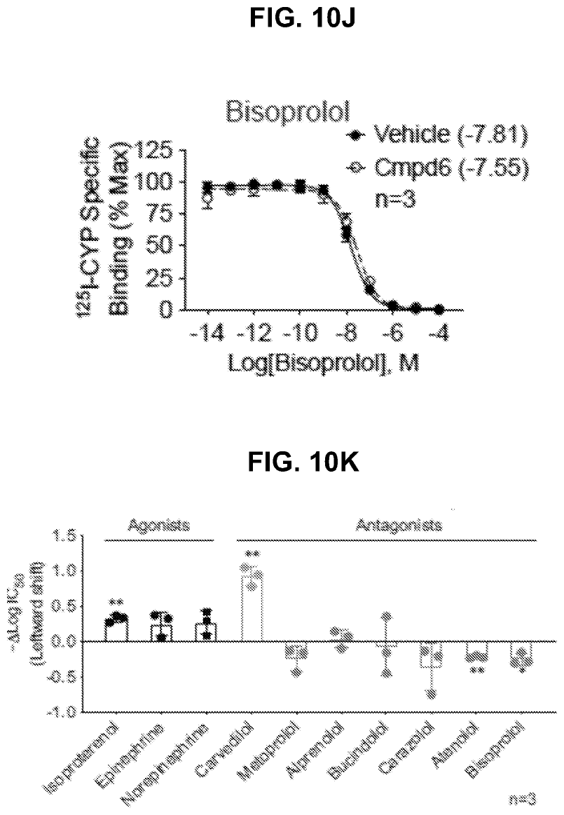

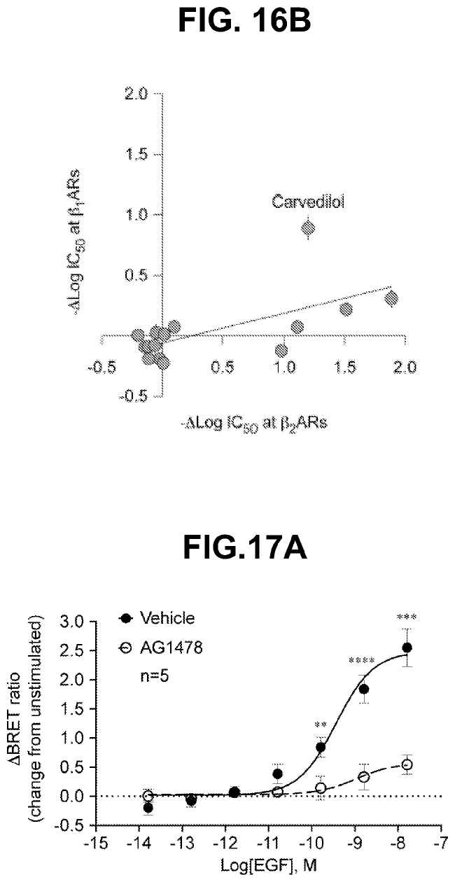

Compound-6 selectively potentiates the binding affinity of carvedilol to the β1AR: Cmpd-6 is an unbiased positive allosteric modulator for the β2AR that substantially enhances the affinity of agonists as well as downstream signaling mediated by either G protein or β-arrestin. Carvedilol, a traditional β-blocker widely used in the treatment of cardiac diseases, has also been identified as a β-arrestin-biased agonist for both the β1AR and the β2AR. Cmpd-6 potentiates β-arrestin1-induced high affinity binding of carvedilol to the β2AR, as well as carvedilol-stimulated β2AR cellular signaling. To determine the allosteric modulatory activity of Cmpd-6 on the β1AR, its effect on the orthosteric binding affinity of a comprehensive ligand panel of agonists and antagonists was tested first by performing competition binding assays against radiolabeled antagonist cyanopindolol (125I-CYP) binding to purified 31ARs reconstituted in high-density lipoprotein (HDL) particles. Remarkably, it was found that Cmpd-6 leads to ˜1-log leftward shift of the carvedilol competition binding curve (FIG. 10A, vehicle: Log IC₅₀=−8.89 [−8.98 to −8.80], Cmpd-6: Log IC₅₀=−9.79 [−9.91 to −9.67], mean [95% confidence intervals]), indicating that Cmpd-6 strongly potentiates the binding affinity of carvedilol to the β1AR. Notably, Cmpd-6 minimally enhanced the binding affinity of the full agonist isoproterenol for β1ARs (FIG. 10B, vehicle: Log IC₅₀=−7.60 [−7.65 to −7.54], Cmpd-6: Log IC₅₀=−7.92 [−8.00 to −7.83]), which is in marked contrast to its effect on β2ARs where it enhanced isoproterenol binding affinity by 50-fold. This marginal effect of Cmpd-6 on isoproterenol affinity for β1ARs is consistent with previous studies. When tested against a diverse ligand panel, it showed minimal positive cooperativity with the full agonists epinephrine and norepinephrine and no positive cooperativity with any other antagonist tested (FIGS. 10C-10J). Overall, Cmpd-6 showed a subtle increase of agonist binding to the β1AR, indicated by a slight leftward shift of competition binding curves and change of IC₅₀ value, and no positive modulation of binding affinity for the 5 other antagonists tested (FIGS. 10C-10K). To compare the Cmpd-6 cooperativity with ligands between β1ARs and β2ARs, in separate experiments, Cmpd-6-induced affinity shifts of an expanded panel of ligands on the β1AR was determined (FIG. 16A), and plotted against the shifts observed for the β2AR shown in FIG. 1D (FIG. 16B). Comparison between receptor subtypes highlights that carvedilol is the only ligand among those tested that show strong cooperativity with Cmpd-6 at both the β1AR and β2AR (FIG. 16B). The binding affinity of Cmpd-6 to carvedilol-bound β1ARs can be calculated from the leftward shift in carvedilol binding affinity by Cmpd-6 (ΔLog IC₅₀) by radioligand competition binding for β1ARs and β2ARs. The binding affinity of Cmpd-6 to carvedilol-bound β1ARs is 17 μM and to carvedilol-bound β2ARs is 1.2 μM. Though traditionally identified and used as a β-blocker, Carvedilol has been shown to possess β-arrestin biased activity by selectively engaging β-arrestins as the transducer in mediating downstream PAR signaling. The discovery that Cmpd-6 specifically potentiates the binding affinity of carvedilol to the β1AR (FIGS. 10A and 10K) indicates that Cmpd-6 is a β-arrestin-biased allosteric modulator of the β1AR.

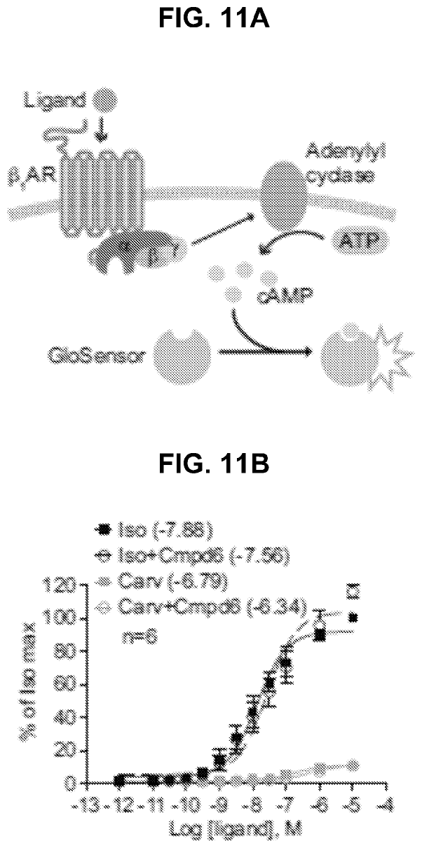

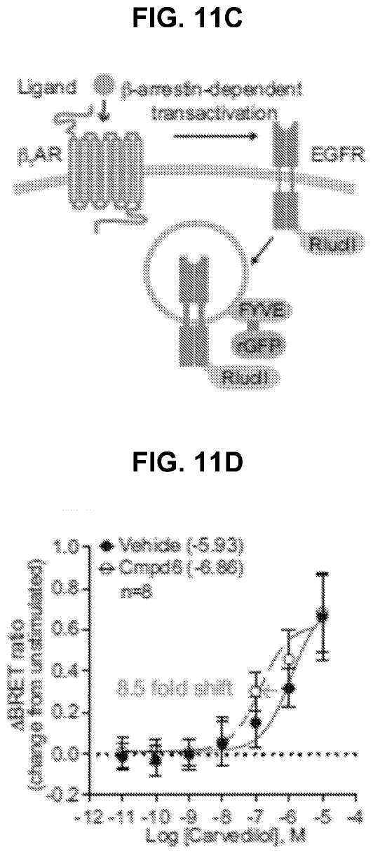

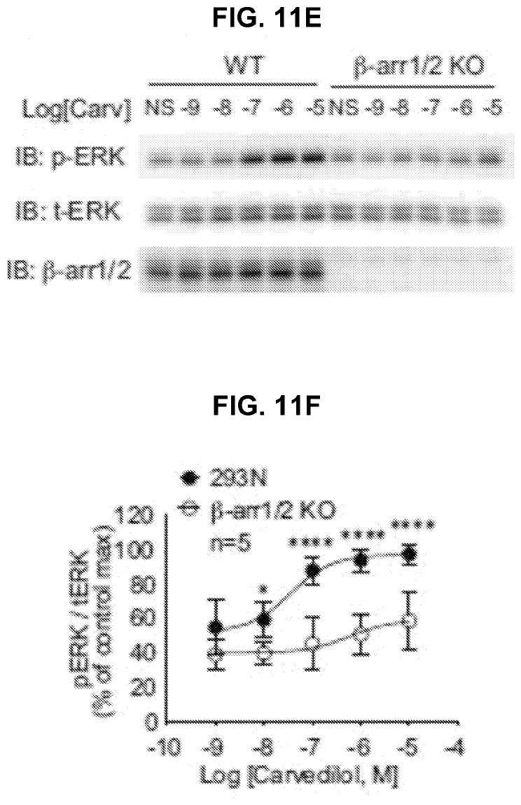

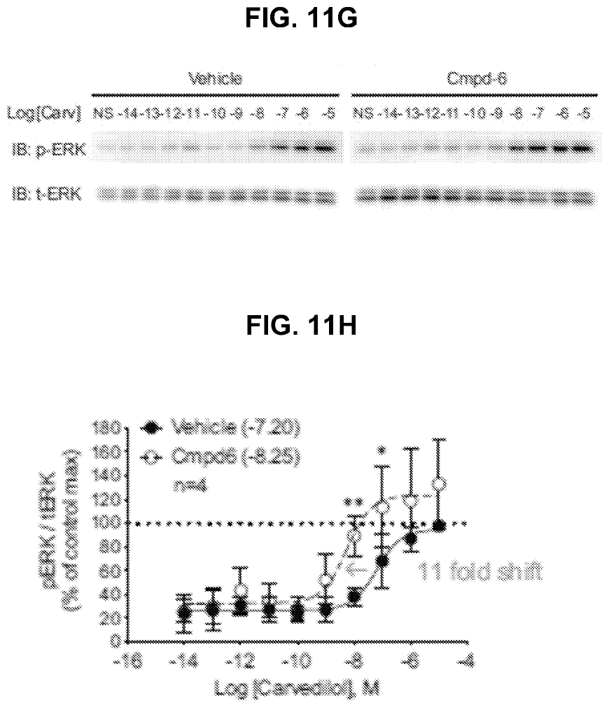

Cmpd-6 shows positive cooperativity on β-arrestin-mediated, but not Gs protein-mediated, signaling induced by carvedilol stimulation of the β1AR. To determine the functional significance of the allosteric modulation activity of Cmpd-6 on β1ARs, the effect of Cmpd-6 on β1AR-mediated cellular signaling in HEK293 cells was tested. First, Gs protein-mediated cAMP generation was monitored in the presence or absence of Cmpd-6. HEK293 cells were transfected with β1ARs and the luciferase-based cAMP biosensor GloSensor (FIG. 11A). Upon ligand stimulation of the β1AR, Gs activation of adenylyl cyclase generates cAMP that is detected by through a conformational change of GloSensor to produce light (FIG. 11A). It was observed that the full agonist isoproterenol robustly induced cAMP production in a dose-dependent manner, while the β-arrestin-biased agonist carvedilol induced a low level of cAMP only at high concentrations of ligand (FIG. 11B). This is consistent with a previous study showing that carvedilol displays a very low potency and efficacy with β1AR-mediated Gs activation in β1AR-overexpressed cells. Importantly, Cmpd-6 showed no enhancement of the dose response curve for either ligand (FIG. 11B), indicating that Cmpd-6 has little or no PAM activity on the Gs-mediated β1AR signaling.

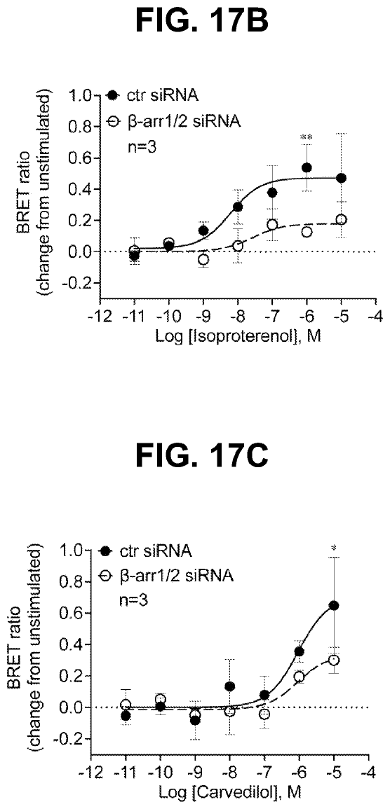

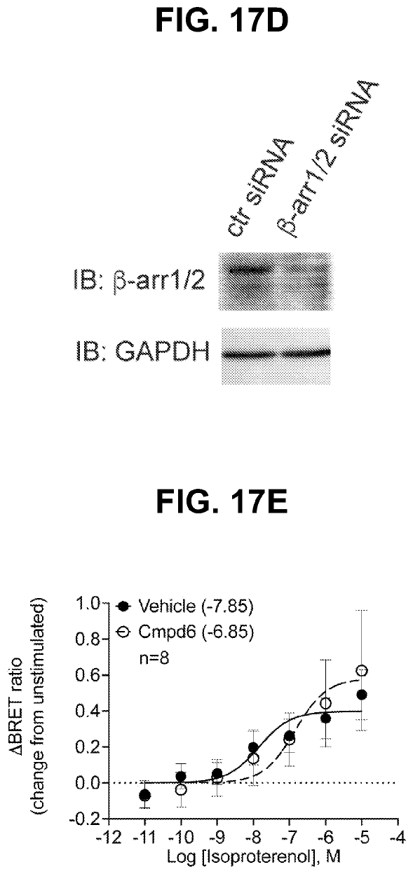

To determine the activity of Cmpd-6 on carvedilol-stimulated β-arrestin-mediated β1AR signaling, the effect of Cmpd-6 on β1AR stimulated EGFR transactivation was tested. Previous studies showed that ligand stimulation of the β1AR induces GRK (G protein-coupled receptor kinase)-mediated receptor phosphorylation and subsequent β-arrestin and Src recruitment. This in turn leads to the activation of matrix metalloproteinases and shedding of HB-EGF that binds to EGFRs promoting its activation and internalization, a process known as EGFR transactivation. To allow sensitive and quantitative detection of β-arrestin-mediated EGFR transactivation, a bioluminescence resonance energy transfer (BRET) sensor was developed using EGFR-RlucII and the early endosome-targeted FYVE-rGFP to monitor the internalization of EGFRs (FIG. 11C). To assess the fidelity of this biosensor assay system, it was shown that EGF ligand induced a robust change in BRET ratio, which was blocked by the EGFR inhibitor AG1478 (FIG. 17A). In β1AR stably expressing HEK293 cells transfected with EGFR-RLucII and FYVE-rGFP, both the full agonist isoproterenol and the β-arrestin-biased agonist carvedilol dose-dependently induce EGFR internalization, and the response is diminished by the siRNA knockdown of β-arrestin1/2 (FIGS. 17B-17D). Cmpd-6 potentiated the carvedilol-induced response, indicated by the leftward shift of carvedilol dose response curve (FIG. 11D, vehicle: log EC₅₀=−5.93 [−6.28 to −5.61], Cmpd-6: log EC₅₀=−6.86 [−7.19 to −6.46]), while it showed no PAM activity on the isoproterenol response (FIG. 17E).

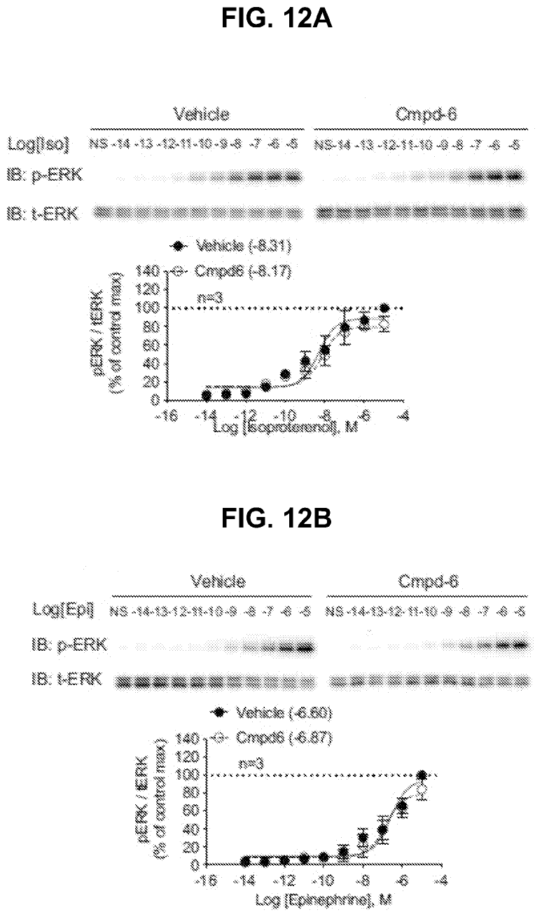

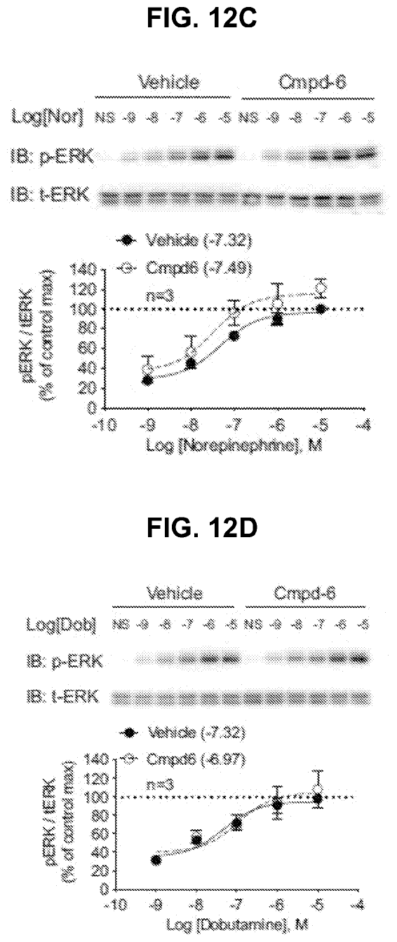

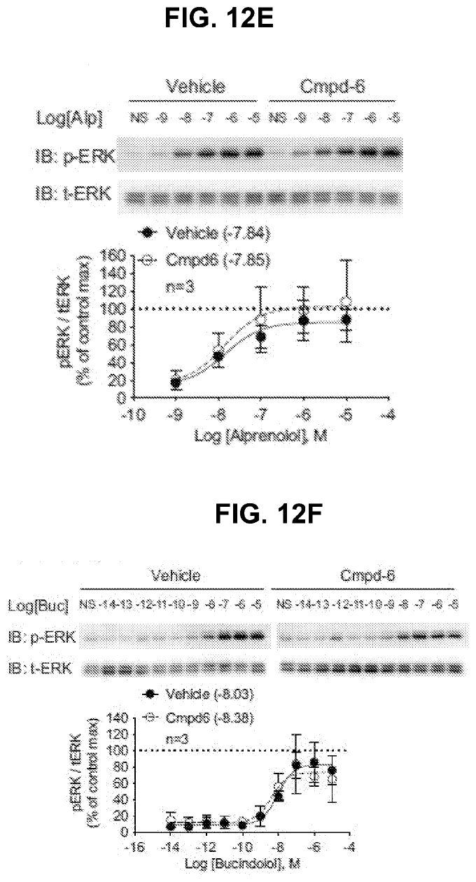

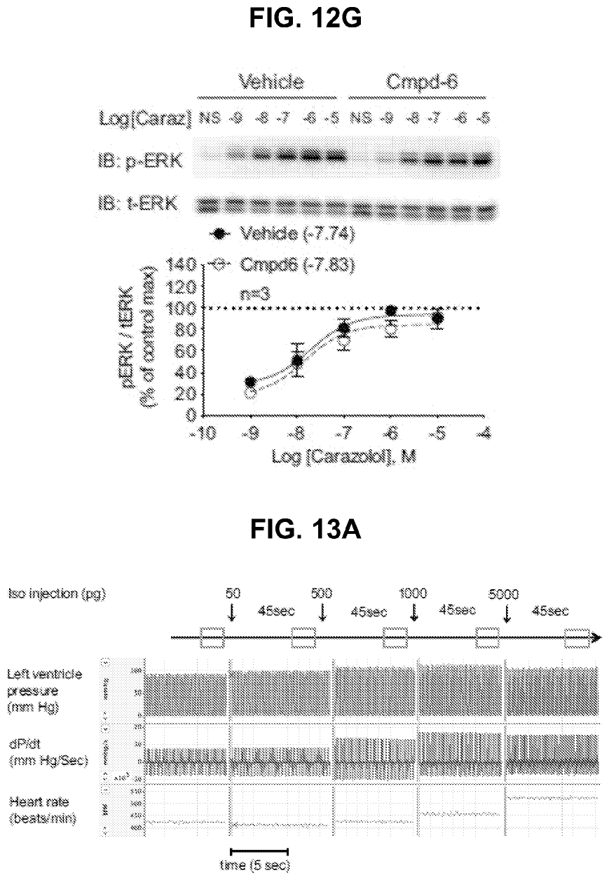

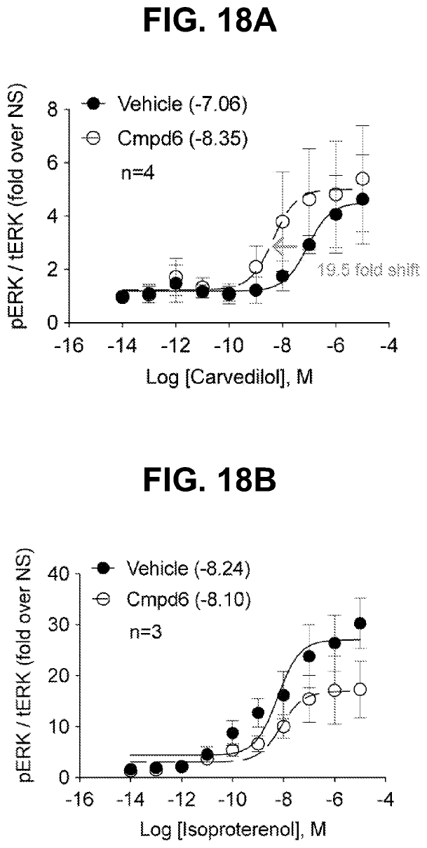

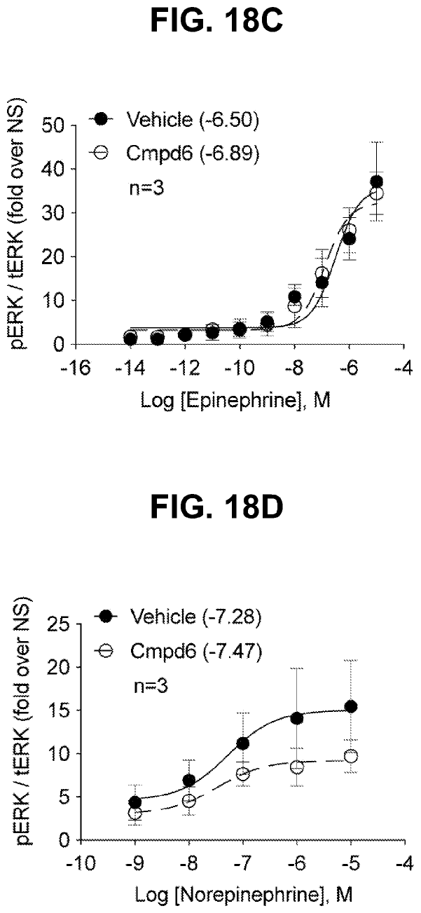

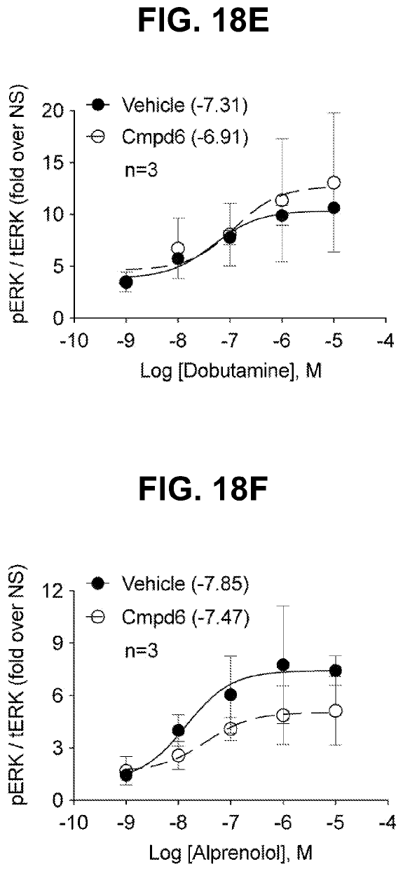

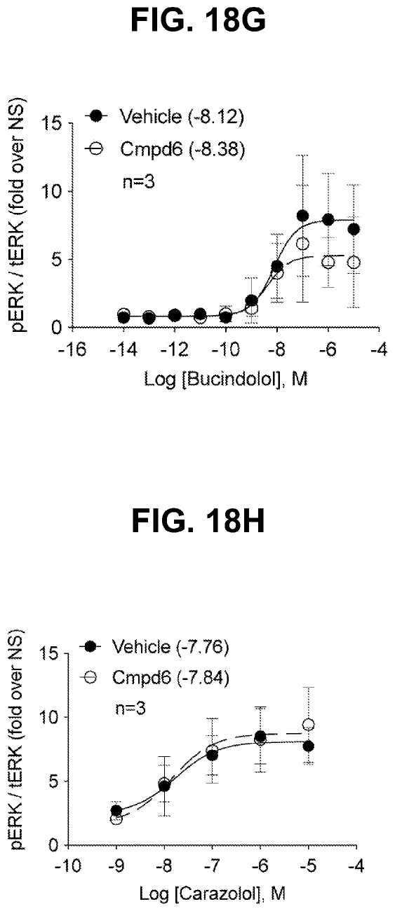

Next, Cmpd-6 in β1AR-mediated ERK (extracellular signal-regulated kinase) activation induced by carvedilol was tested. It has been previously shown that carvedilol induces β1AR-mediated ERK signaling in a β-arrestin-dependent manner (Kim et al., Proc Natl Acad Sci USA 105(38): 14555-14560 (2008); Luttrell et al., Sci Signal 11(549) (2018); Wang et al., Nat Commun 8(1): 1706 (2017)). Consistent with these earlier studies, it was shown that carvedilol-induced ERK activation is largely lost in the HEK293 cells in which the genes for both β-arrestin1 and 2 are deleted through CRISPR gene editing (FIGS. 11E and 11F). The low level of ERK activation in β-arrestin1/2 knockout cells is likely the result of some activation of Gs at high concentrations of carvedilol (FIG. 11B). In marked contrast to the effect observed on Gs activation, Cmpd-6 substantially potentiates carvedilol-induced ERK activation as indicated by an 11-fold leftward shift in the ERK dose response curve along with a small increase in the maximal response (FIGS. 11G and 11H, vehicle: log EC₅₀=−7.20 [−7.44 to −6.98], Cmpd-6: log EC₅₀=−8.25 [−8.68 to −7.85]). In addition, Isoproterenol stimulation showed no cooperativity by Cmpd-6 (FIG. 12A). Expanding testing to a broad panel of ligands, it was found that the effect of Cmpd-6 is highly selective to carvedilol with minimal or no augmentation of ERK phosphorylation in response to stimulation by a comprehensive panel of PAR ligands (FIGS. 12A-12G). Data of the ERK activation assay is also presented as fold over non-stimulated samples of each treatment (FIGS. 18A-18H).

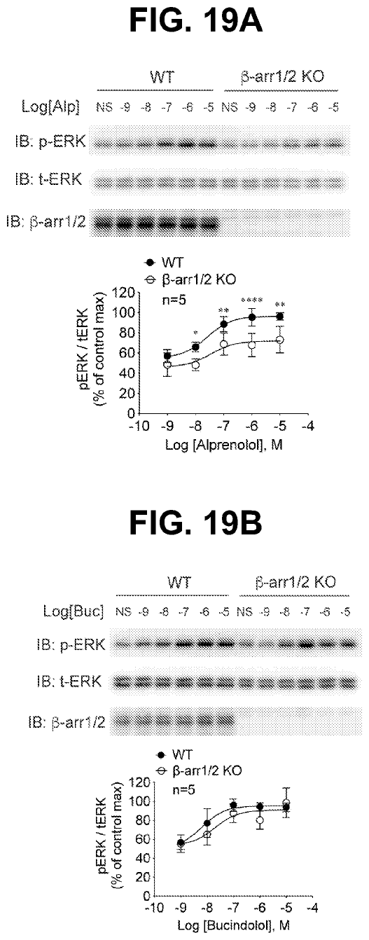

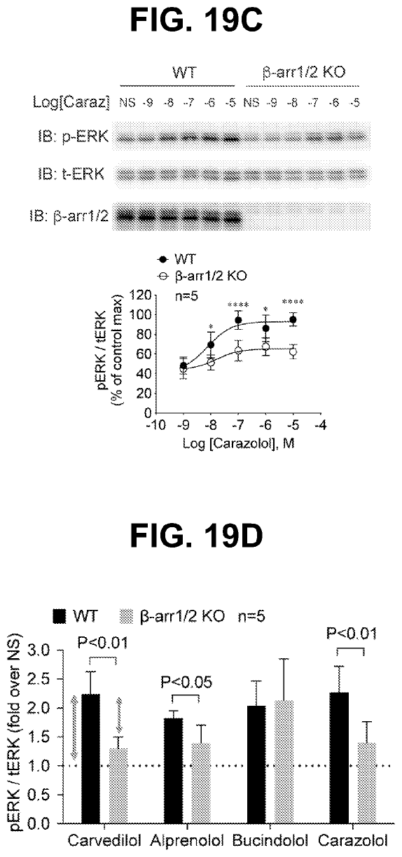

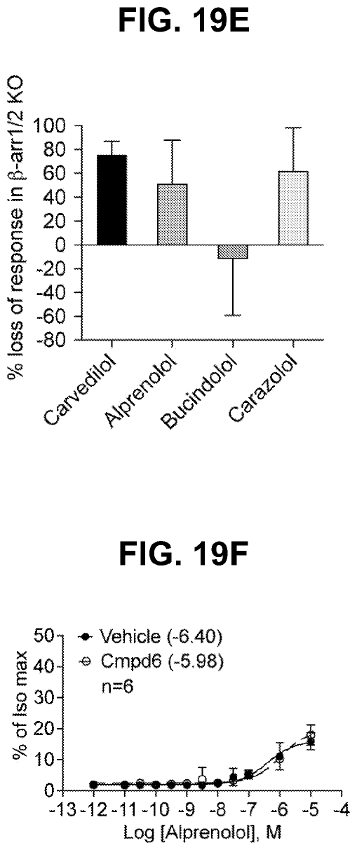

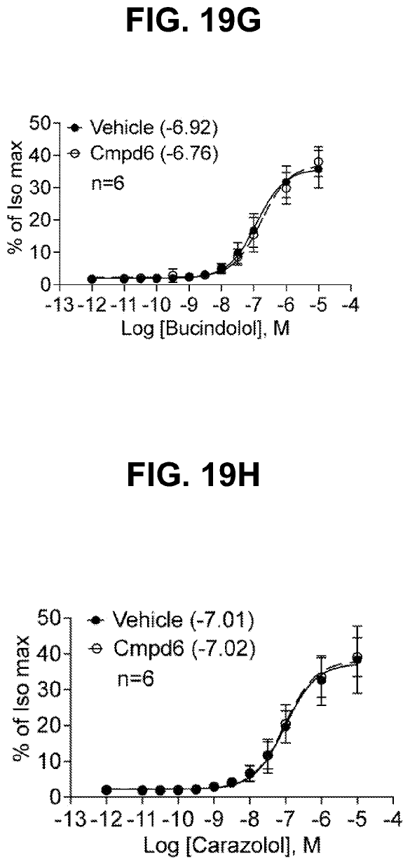

Interestingly, in an expanded ligand panel on ERK activation, it was noted that a number of other β-blockers robustly induced β1AR-mediated ERK activation (FIGS. 12E-12G). To dissect the mechanism for the various β-blockers on β1AR signaling, the level of β-arrestin dependency on ERK signaling and Gs activation was determined. In contrast to carvedilol, which activates ERK mainly through β-arrestin (FIG. 11H), the activation induced by alprenolol and carazolol are partially (˜50%) dependent on β-arrestin, while bucindolol activates ERK independent of β-arrestin (FIGS. 19A-19E). Then, GloSensor assays of Gs-mediated cAMP generation were performed and showed that bucindolol and carazolol induced robust activation of cAMP (40% of the maximal isoproterenol response), while alprenolol only modestly (20% of the maximal isoproterenol response) and metoprolol induced no response (FIG. 19F). Importantly, none of the β-blockers tested had positive cooperativity to Cmpd-6 (FIG. 19F). These data show that while a number of β-blockers can activate β1AR signaling, they differentially engage signal transducers to activate downstream pathways as indicated by their differential cooperativity with Cmpd-6, dependency on β-arrestin, and activity on Gs-mediated cAMP production. Taken together, these findings show the selective positive cooperativity of Cmpd-6 for the β1AR with carvedilol with respect to binding affinity and the selective enhancement of carvedilol-stimulated β-arrestin-mediated signaling, but not Gs-mediated cAMP production. These data indicate that Cmpd-6 is a β-arrestin-biased PAM of the carvedilol-occupied β1ARs.

Cmpd-6 potentiates β-arrestin-dependent cell survival by carvedilol in response to ischemia reperfusion myocardial injury: Data from in vitro and cellular assays strongly supports that Cmpd-6 selectively cooperates with carvedilol to enhance β-arrestin-biased β1AR signaling. Previous studies have shown that sustained G-protein activation by β1ARs is associated with deleterious cardiac remodeling while β-arrestin signaling is potentially cardioprotective. While carvedilol, among other β-blockers, is considered as a standard therapy in heart failure, a major limitation is that it is often difficult to achieve the maximally tolerated dose due to the development of adverse effects. Carvedilol may lead to fatigue and impairs functional capacity by virtue of its blockade of the catecholamine-stimulated heart rate response during exercise. Therefore, it was reasoned that a β-arrestin-biased PAM for the β1AR, such as Cmpd-6 as identified herein, could potentially enhance the cardioprotective effect of carvedilol in vivo while minimizing the hemodynamic perturbation.

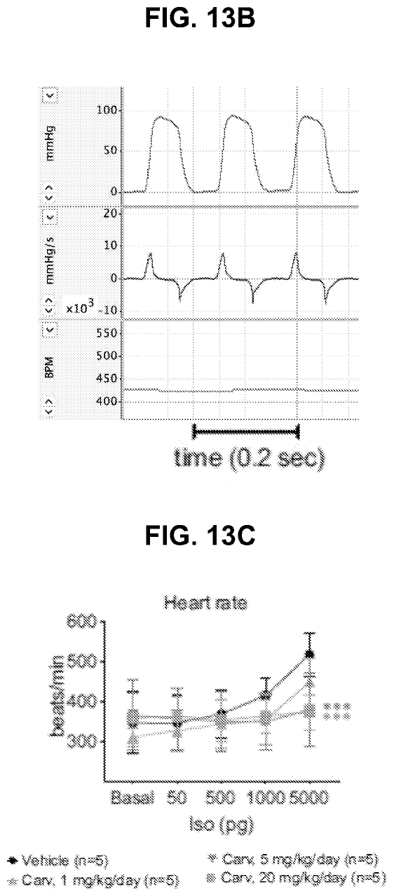

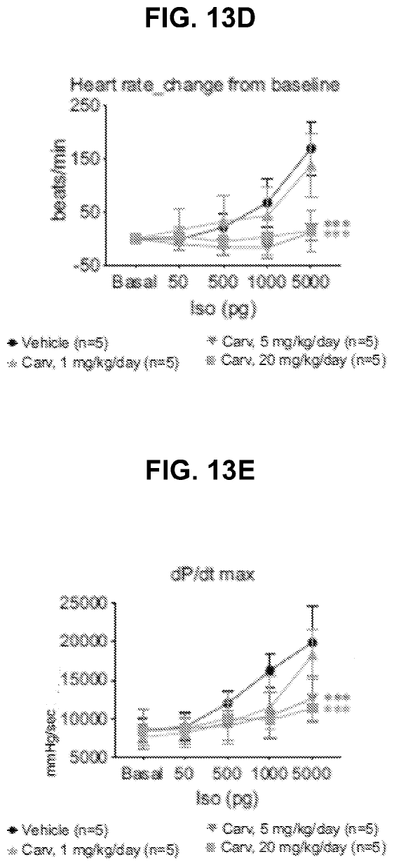

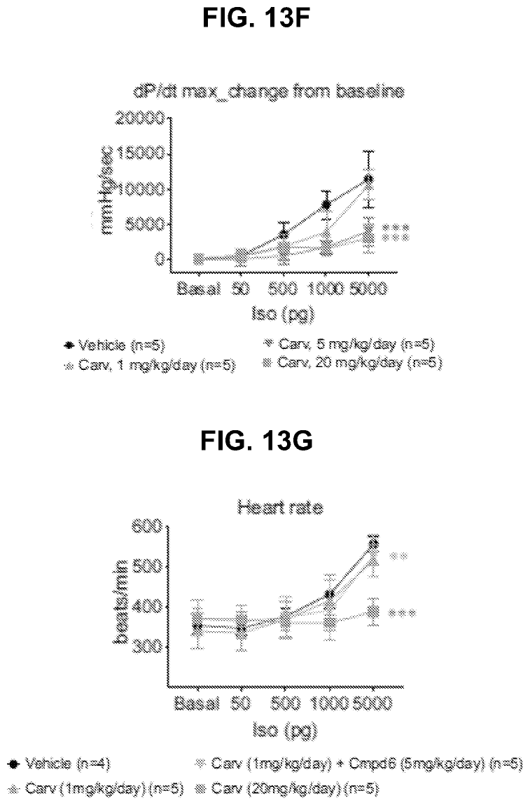

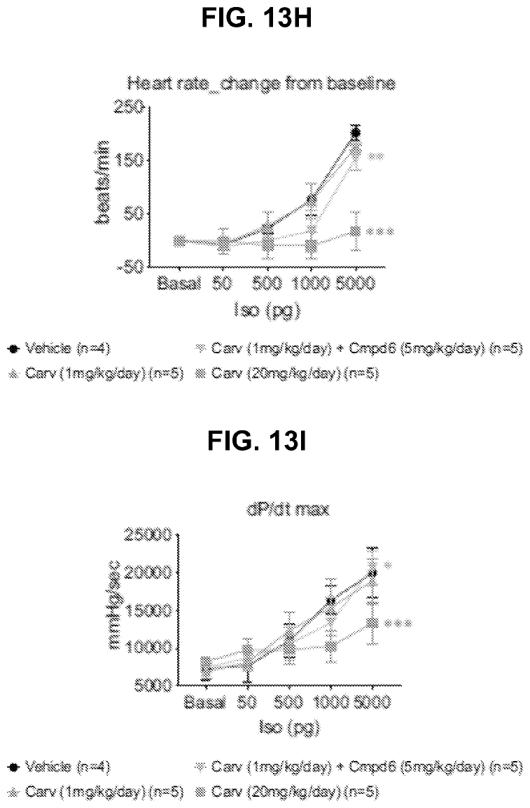

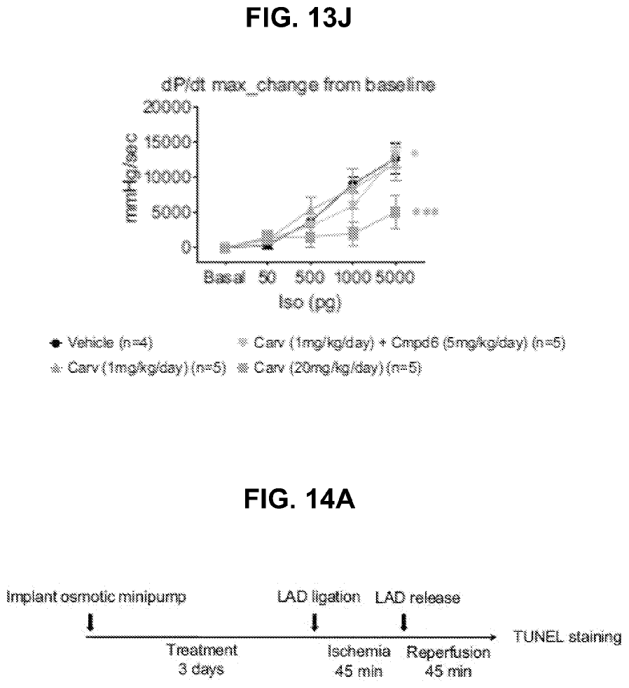

To test this hypothesis, the in vivo β-blockade effect of carvedilol on catecholamine-induced cardiac hemodynamics was determined by assessing the response to isoproterenol on heart rate and contractility in mice pretreated with increasing doses of carvedilol. Mice were treated with 1, 5 or 20 mg/kg/day carvedilol with Alzet osmotic minipump for 3 days, then intravenously injected boluses of increasing doses of isoproterenol while monitoring the hemodynamics with a high fidelity micromanometer catheter inserted retrograde into the left ventricle (FIGS. 13A and 13B). Isoproterenol dose-dependently increased cardiac contractility, indicated by an increase in dP/dt max, as well as heart rate (FIGS. 13A, 13B, and 13C-13F). Low dose of carvedilol at 1 mg/kg/day had minimal effect on isoproterenol-induced hemodynamics, while higher doses of carvedilol blocked both the heart rate and contractility response (FIGS. 13C-13F). Co-administration of Cmpd-6 (5 mg/kg/day) in a ˜3:1 molar ratio to carvedilol (1 mg/kg/day) showed only a modest potentiation of the inhibitory effect on heart rate and dP/dt max (FIGS. 13G-13J). Importantly, the β-blocker effect of low dose carvedilol (1 mg/kg/day) together with Cmpd-6 is substantially smaller than that of higher doses of carvedilol alone (FIGS. 13C-13F and 13G-13J), despite the 7.9-fold enhancement on receptor binding affinity of carvedilol in the presence of Cmpd-6 as shown in FIG. 10A. The lack of a rightward shift of the dose response curve by Cmpd-6 suggests that it has minimal regulatory effects in vivo on β1AR-mediated Gs signaling.

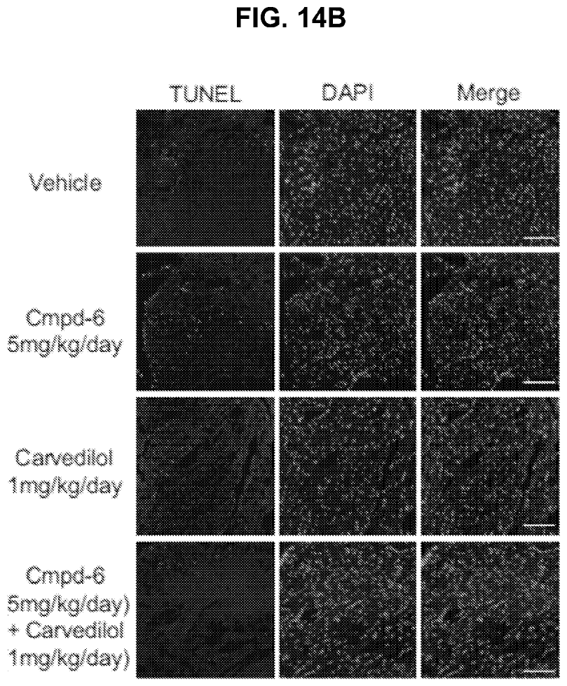

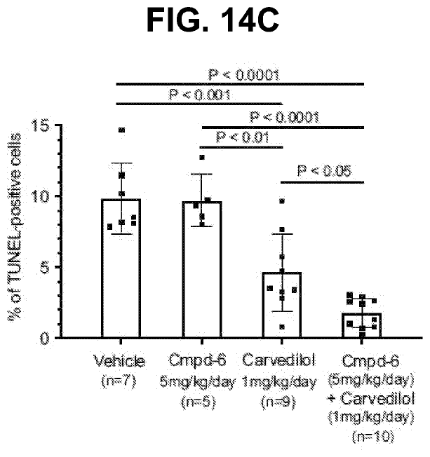

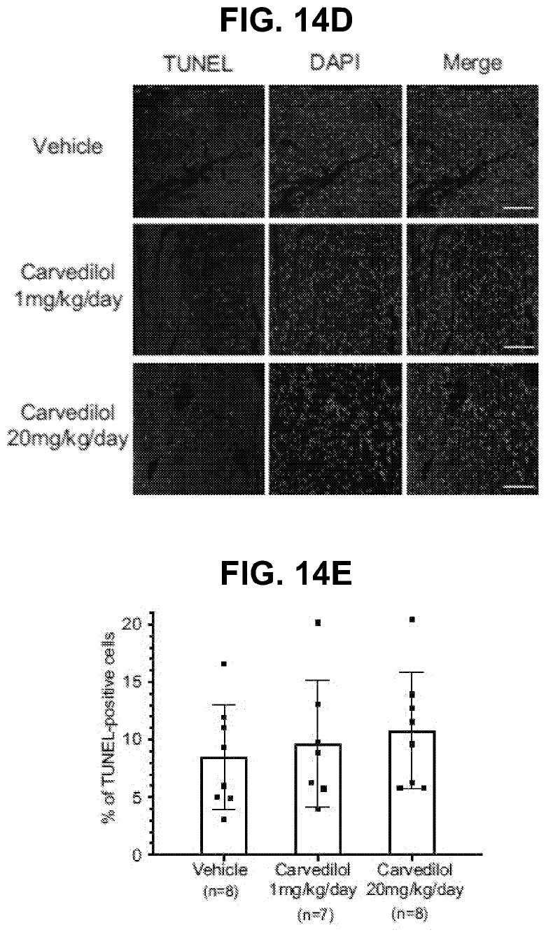

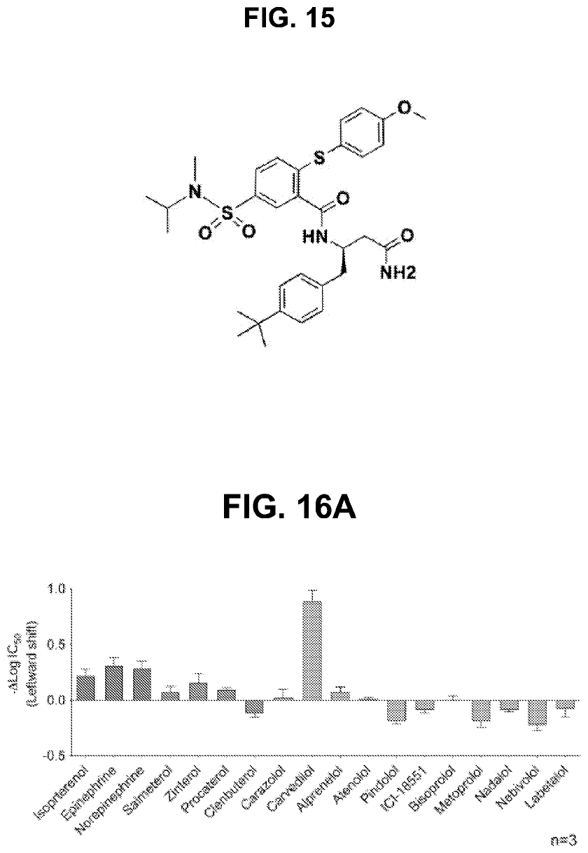

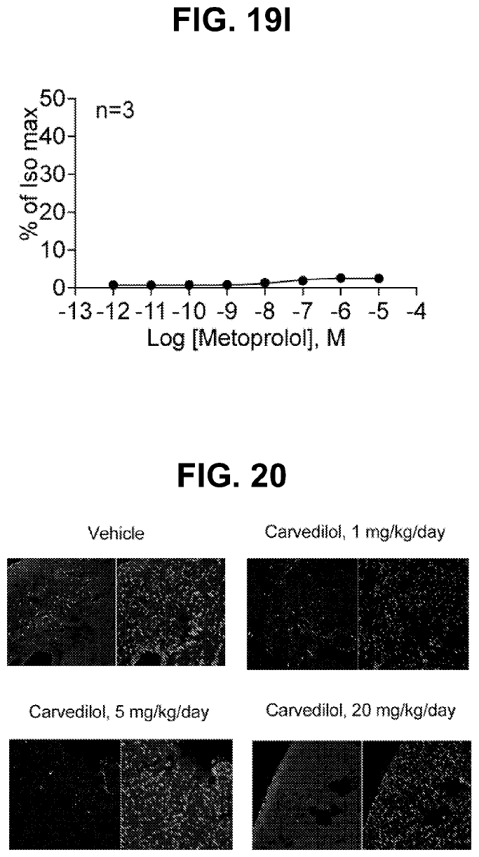

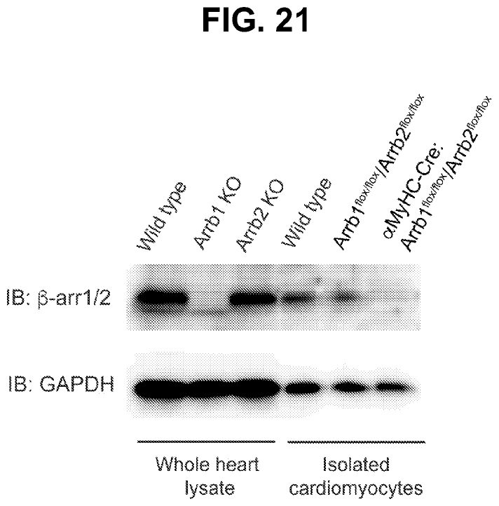

Based on the foregoing data, it was tested whether the positive cooperative effect of Cmpd-6 on low dose carvedilol could potentiate its action on cell survival in response to ischemia/reperfusion (I/R) cardiac injury. Wild type mice were treated with vehicle, Cmpd-6 (5 mg/kg/day), carvedilol (1 mg/kg/day), or co-administration of both compounds through Alzet osmotic pump for 3 days to reach the steady state. The left anterior descending (LAD) coronary artery was occluded for 45 mins to cause myocardial ischemia and then released for 45 mins of tissue reperfusion (FIG. 14A). The 1 mg/kg/day dose of carvedilol was chosen, since this dose was sufficient to provide partial cardioprotection in response to I/R injury (FIG. 20), while only minimally enhancing the β-blockade effect on cardiac hemodynamics (FIGS. 13C-13F). In contrast, although higher doses of carvedilol (5 or 20 mg/kg/day) inhibited I/R induced apoptosis (FIGS. 20 and 21), it also showed high level of competitive antagonism (i.e., β-blocker activity) to isoproterenol-stimulated, Gs protein-mediated hemodynamics (FIGS. 13C-13F). Cmpd-6 alone has no intrinsic activity on I/R-induced apoptosis as shown by the ˜10% TUNEL positive cells in the ischemic zone in mice pretreated with either vehicle or Cmpd-6 (FIGS. 14B and 14C). Carvedilol decreased the level of I/R-induced apoptosis compared to vehicle-treated animals to −5% albeit with considerable variability. In marked contrast, the addition of Cmpd-6 to the same dose of carvedilol substantially and more consistently enhanced the anti-apoptotic effect, indicated by the reduction in the level of TUNEL positive cells to ˜2% with many hearts showing very low levels of injury (FIGS. 14B and 14C).

To determine whether the anti-apoptotic effect of carvedilol is mediated through 3-arrestins, carvedilol was tested in mice with cardiomyocyte-specific deletion of β-arrestin1/2 achieved by α-myosin heavy chain (αMyHC) promoter-driven Cre recombinase (αMyHC-Cre:Arrblflox/flox/Arrb2flox/flox, FIG. 21). Pretreatment with either low or high dose carvedilol did not reduce I/R-induced apoptosis in the β-arrestin1/2 knockout animals (FIGS. 14D and 14E), indicating that the cardioprotective action of carvedilol in vivo is mediated by signals downstream of β-arrestin. Taken together, these data support the contention that the β-arrestin-biased β1AR PAM Cmpd-6 enhances the in vivo cardioprotective effects of carvedilol against cardiac injury-induced cell apoptosis, and further supports Cmpd-6 as having a potential therapeutic benefit in enhancing the effectiveness of carvedilol under conditions of cardiac injury.

### D. Discussion

Herein, Cmpd-6 is shown to be a β-arrestin-biased positive allosteric modulator for β1ARs occupied with carvedilol. Cmpd-6 was identified as a PAM for the β2AR that enhances agonist binding affinity and potentiates both the Gs protein- and β-arrestin-dependent signaling. Cmpd-6 was initially shown to cooperate with carvedilol, but not other β-blockers, on the β2AR. This prompted the assessment of its properties at the β1AR. Herein, Cmpd-6 is shown to selectively enhance the binding affinity of the β-arrestin-biased agonist carvedilol to the β1AR, while having minimal effects on the affinity of other agonists and antagonists tested. Cmpd-6 also potentiates carvedilol-induced β-arrestin-dependent β1AR signaling including EGFR transactivation and ERK activation, whereas having no effect on Gs protein-activated cAMP generation. In contrast to its minimal effect on the in vivo β-blockade function of carvedilol on catecholamine-induced cardiac hemodynamics, Cmpd-6 augments the cardioprotective roles of carvedilol against myocardial ischemia/reperfusion-induced apoptosis, which is shown herein to be a β-arrestin-dependent process since the anti-apoptotic effect of carvedilol is abolished in mice with cardiac-specific deletion of β-arrestin1/2. The highly selective cooperativity of Cmpd-6 with carvedilol on ligand binding affinity to the β1AR, β-arrestin-biased cellular signaling, as well as the β-arrestin-dependent anti-apoptotic role in vivo, suggests that Cmpd-6 is a β-arrestin-biased PAM for the β1AR and may have therapeutic potential to enhance the clinical effects of carvedilol that is widely used in the treatment of cardiac diseases.

β1- and β2ARs are the most abundant GPCRs expressed in mammalian hearts and are principal regulators of cardiac pathophysiology. Prolonged catecholamine stimulation leads to cardiac injury and cardiomyocyte apoptosis, and mice with cardiac-specific β1AR overexpression develop myocyte hypertrophy and cardiac dysfunction, indicating that excessive catecholamine activation of β1ARs is pathogenic to the heart. In contrast, β-arrestin-mediated β1AR signaling appears to provide cardioprotection. Herein, it is shown that carvedilol protects hearts from ischemia/reperfusion-induced apoptosis, and Cmpd-6 enhances this anti-apoptotic effect. Importantly, it is also demonstrated that the anti-apoptotic effect of carvedilol in cardiomyocyte is, at least in part, mediated through β-arrestin driven signaling pathways. In contrast to the predominant myocyte expression and the pro-apoptotic effect of the β1ARs, β2ARs are primarily expressed on fibroblasts and endothelial cells, and have been shown to be cardioprotective from apoptosis. Based on the predominant expression of β1ARs in myocytes, and the opposing effect of agonist-activated β1AR and β2AR on apoptosis, it was reasoned that the protective effects of carvedilol and Cmpd-6 against ischemia/reperfusion-induced apoptosis is most likely to be mediated by the β1AR. However, a previous study showed that the β2AR modulator pepducin ICL1-9 reduces I/R-induced cardiomyocyte death in a β2AR- and β-arrestin-dependent manner. Therefore, it is possible that the enhanced anti-apoptotic effect of Carvedilol by Cmpd-6 also involves the β2AR. The precise dissection of the contribution of receptor subtypes would require testing carvedilol and Cmpd-6 in β1AR- or β2AR-knockout animals.

In regard to the mechanism of action for the in vivo cardioprotection by carvedilol, it may be speculated that the enhancement by Cmpd-6 on carvedilol-stimulated PAR-β-arrestin signaling plays an important role. β1AR-mediated EGFR transactivation is β-arrestin dependent and confers cardioprotection against myocardial apoptosis induced by chronic catecholamine stimulation. β1AR-mediated EGFR transactivation stimulates differential subcellular activation of ERK and Akt to modulate caspase 3 activity and apoptotic gene expression. It has also been shown that β-arrestin mediates angiotensin II type 1 receptor (AT1R)-induced EGFR/ERK transactivation and protects mouse hearts against mechanical stress-induced apoptosis. AT1R stimulation with the β-arrestin-biased agonist, SII, activates both ERK/p90RSK and PI3K/AKT pathways leading to inactivation of the pro-apoptotic protein BAD to protects cells from oxidative stress-induced apoptosis. Furthermore, the anti-apoptotic effects of Cmpd-6 and carvedilol may also be mediated by regulation of microRNA (miR) processing. Carvedilol-stimulated 31AR-activated β-arrestin1 promotes the processing of a subset of miRs in the mouse heart, among which miR-125b-5p reduced expression of pro-apoptotic genes bak1 and klfl3 in cardiomyocytes to protect mouse heart from ischemic injury. Finally, the β-arrestin-biased β2AR modulator pepducin ICL1-9 protects against I/R-induced cardiac injury and cardiomyocyte death through activating the RhoA/ROCK pathway and reducing mitochondrial oxidative stress.

Based on the central role of βARs in the heart, β-blockers are first-line agents for the treatment of cardiac diseases such as heart failure. However, different β-blockers have variable therapeutic effectiveness in treating heart failure. Advances in understanding the complexities of GPCR biology and detailed dissections of pharmacological actions of β-blockers may assist in understanding their differential efficacy. Herein, it is shown that the β-blockers alprenolol, carvedilol, bucindolol and carazolol differentially engage Gs and 3-arrestin to activate downstream signaling. The unique properties of carvedilol in stimulating 3-arrestin-dependent β1AR signaling may contribute to its potential clinical superiority as suggested by meta-analyses showing that carvedilol has lowest all-cause mortality among different β-blockers tested in heart failure.

While β-blockers are considered standard of therapy in heart failure, the level of patient unresponsiveness to β-blocker treatment and intolerable adverse effects support a quest to develop novel PAR ligands with improved drug efficacy and safety. Recent developments in identifying allosteric modulators for GPCRs may offer great potential as novel therapeutics as they have the potential for greater receptor subtype specificity, fewer off-target side effects and better drug safety profiles than orthosteric ligands. Cmpd-6 was identified by a DNA-encoded small molecule library affinity screen for agonist-bound β2ARs. Cmpd-6 potentiates both Gs-mediated cAMP generation and β-arrestin recruitment to β2ARs with comparative efficacy in HEK293 cells, indicating its unbiased PAM activity on β2ARs. Cmpd-6 also cooperates with the β-arrestin-biased agonist carvedilol, but not other β-blockers, on β2ARs. In contrast to its ˜50-fold enhancement of isoproterenol binding affinity for β2ARs, Cmpd-6 enhances isoproterenol binding affinity for β1ARs by only 2-fold as shown in this study as well as previous ones. The selectivity of Cmpd-6 on agonist binding affinity for β2ARs over β1ARs might be due to the structural differences between the two receptor subtypes in the allosteric binding pocket for Cmpd-6.

Mutational studies introducing key β2AR residues interacting with Cmpd-6 into the corresponding β1AR sites created gain-of-function mutant β1ARs, to which Cmpd-6 salvaged its PAM behavior for agonist binding. Interestingly, in contrast to its marginal effect on agonist binding to β1ARs, Cmpd-6 enhances the affinity of the antagonist carvedilol to β1ARs by ˜8 fold leading to a potentiation of β-arrestin signaling. This property of Cmpd-6 on carvedilol, and not any other antagonist tested, is likely based on structural determinants whereby Cmpd-6 binding to carvedilol-occupied β1ARs or β2Ars stabilizes the receptor to adopt a “β-arrestin” conformation thereby engaging β-arrestin, and not Gs, as its preferred signaling transducer.

Data herein demonstrate that Cmpd-6 potentiates the β-arrestin-dependent anti-apoptotic effects of carvedilol in vivo while having minimal effect on blocking the physiological response to catecholamines. Major side effects of carvedilol treatment in heart failure patients are exertional intolerance, dizziness and advanced heart block due to sympathetic activity attenuation. Accordingly, allosteric modulators such as Cpmd-6 could provide important therapeutic advantages by enhancing the cardioprotective action of carvedilol, but minimizing the adverse effects often found with high dose carvedilol. In the future, determining the effects of Cmpd-6 in chronic left ventricle dysfunction will be of great interest to further evaluate the therapeutic potential of this compound. In addition, the structure-activity relationship study of Cmpd-6 may lead to identification of new compounds with desirable receptor subtype selectivity or signaling modulation effects. For instance, a chemically modified analog of Cmpd-6 selectively cooperates with carvedilol on the β2AR while showing no PAM activity on agonists, which is similar to the effects of Cmpd-6 on the β1AR. Based on the differential functional consequences of β1AR and β2AR activation in the heart, further development of Cmpd-6 analogs with high receptor selectivity and selective carvedilol cooperativity may provide additional therapeutic benefits.

In conclusion, Cmpd-6 is a β-arrestin-biased PAM for β1ARs. It selectively increases the affinity of the β-arrestin-biased agonist carvedilol for β1ARs and potentiates β-arrestin-mediated signaling stimulated with carvedilol. Importantly, Cmpd-6 enhances the β-arrestin-dependent in vivo effects of carvedilol to protect hearts from ischemia/reperfusion injury-induced apoptosis. Data herein suggest that the development of β-arrestin-biased PAR allosteric modulators may provide a new direction for improving current treatments or developing novel agent.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A method of enhancing the effectiveness of a beta-arrestin-biased β-blocker being administered to a subject, the method comprising administering to the subject a therapeutically effective amount of a compound according to Formula (I): 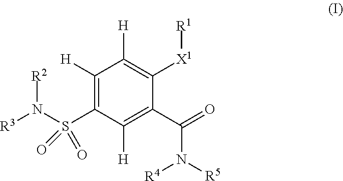
wherein:
R¹ is an aryl group optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆ alkyl)₂ —OC₃₋₆cycloalkyl, —NHC₃₋₆cycloalkyl, —N(C₁₋₆alkyl)(C₃₋₆cycloalkyl), and —N(C₃₋₆cycloalkyl)₂, and optionally the aryl is phenyl wherein two substituents join to form a 5- to 7-membered non-aromatic fused ring containing 1-2 heteroatom groups selected from NR^{1a} and O;
X₁ is O, N(H), N(C₁₋₄alkyl), S, S(O), S(O)₂, C(O), or CR^{1b}R^{1c};
R^{1a} is H or C₁₋₄alkyl;
R^{1b} and Ric are each independently hydrogen or C₁₋₄alkyl, or R^{1b} and Ric together with the carbon to which they are attached form a C₃₋₆cycloalkyl ring;
R² is hydrogen, C₁₋₆alkyl, or C₃₋₇cycloalkyl;
R³ is hydrogen, C₁₋₆alkyl, C₃₋₇cycloalkyl, or aryl, the aryl being optionally substituted with 1-5 substituents independently selected from the group consisting of C₁₋₆alkyl, C₁₋ₑhaloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂; alternatively, R² and R³ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R⁴ is hydrogen, C₁₋₆alkyl, or C₃₋₇cycloalkyl;
R⁵ is CHR^{5a}R^{5b}; alternatively, R⁴ and R⁵ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R^{5a} is aryl or —C₁₋₃alkylene-aryl, wherein each aryl in R^{5a} is optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆ alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂;
R^{5b} is X² or —C₁₋₃alkylene-X²; and
X² is —CN, —C(O)OH, —C(O)OC₁₋₄alkyl, —C(O)NH₂, —C(O)NHC₁₋₄alkyl, —C(O)N(C₁₋₄alkyl)₂, —SO₂NH₂, —SO₂NHC₁₋₄alkyl, or —SO₂N(C₁₋₄alkyl)₂. (Cmpd 6), or a pharmaceutically acceptable salt, solvate, hydrate, prodrug, or derivative thereof, or a pharmaceutical composition thereof, and
a β-arrestin-biased β-blocker such that the effectiveness of the β-arrestin-biased p-blocker is enhanced.

2. The method of claim 1, wherein the β-arrestin-biased beta blocker is administered to treat and/or prevent high blood pressure in the subject.

3. The method of claim 1, wherein the β-arrestin-biased beta blocker is administered to treat and/or prevent heart disease in the subject.

4. The method as in any of the preceding claims, wherein the β-arrestin-biased β-blocker is carvedilol (Coreg™).

5. The method as in any of the preceding claims, wherein the β-arrestin-biased β-blocker is administered prior to the compound according to Formula (I).

6. The method as in any of claims 1-4, wherein the β-arrestin-biased β-blocker is administered concurrently with the compound according to Formula (I).

7. The method as in any of claims 1-4, wherein the β-arrestin-biased β-blocker is administered after the compound according to Formula (I).

8. The method of any one of the preceding claims, wherein the compound according to Formula (I) is Cmpd 6, having the formula: 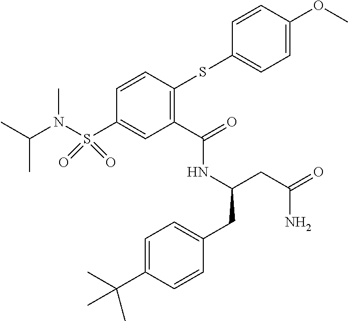 [SMILES: COc1ccc(Sc2ccc(S(=O)(=O)N(C)C(C)C)cc2C(=O)N[C@@H](CC(N)=O)Cc2ccc(C(C)(C)C)cc2)cc1]
or a pharmaceutically acceptable salt, solvate, hydrate, prodrug, or derivative thereof, or a pharmaceutical composition thereof.

9. The method of any of the preceding claims, wherein the compound according to Formula (I) is Cmpd A9, having the formula: 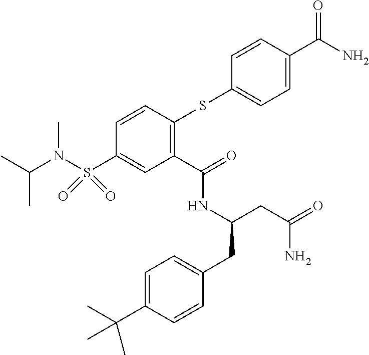 [SMILES: CC(C)N(C)S(=O)(=O)c1ccc(Sc2ccc(C(N)=O)cc2)c(C(=O)N[C@@H](CC(N)=O)Cc2ccc(C(C)(C)C)cc2)c1]
or a pharmaceutically acceptable salt, solvate, hydrate, prodrug, or derivative thereof, or a pharmaceutical composition thereof.

10. A method of treating and/or preventing high blood pressure or heart disease in a subject, the method comprising administering a therapeutically effective amount of a compound according to Formula (I): 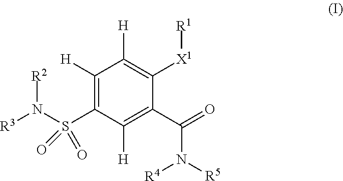
wherein:
R¹ is an aryl group optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆ alkyl)₂ —OC₃₋₆cycloalkyl, —NHC₃₋₆cycloalkyl, —N(C₁₋₆alkyl)(C₃₋₆cycloalkyl), and —N(C₃₋₆cycloalkyl)₂, and optionally the aryl is phenyl wherein two substituents join to form a 5- to 7-membered non-aromatic fused ring containing 1-2 heteroatom groups selected from NR^{1a} and O;
X₁ is O, N(H), N(C₁₋₄alkyl), S, S(O), S(O)₂, C(O), or CR^{1b}R^{1c};
R^{1a} is H or C₁₋₄alkyl;
R^{1b} and Ric are each independently hydrogen or C₁₋₄alkyl, or R^{1b} and Ric together with the carbon to which they are attached form a C₃₋₆cycloalkyl ring;
R² is hydrogen, C₁₋₆alkyl, or C₃₋₇cycloalkyl;
R³ is hydrogen, C₁₋₆alkyl, C₃₋₇cycloalkyl, or aryl, the aryl being optionally substituted with 1-5 substituents independently selected from the group consisting of C₁₋₆alkyl, C₁₋ₑ haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂; alternatively, R² and R³ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R⁴ is hydrogen, C₁₋₆alkyl, or C₃₋₇cycloalkyl;
R⁵ is CHR^{5a}R^{5b}; alternatively, R⁴ and R⁵ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R^{5a} is aryl or —C₁₋₃alkylene-aryl, wherein each aryl in R^{5a} is optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆ alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆ cycloalkyl)₂;
R^{5b} is X² or —C₁₋₃alkylene-X²; and
X² is —CN, —C(O)OH, —C(O)OC₁₋₄alkyl, —C(O)NH₂, —C(O)NHC₁₋₄alkyl, —C(O)N(C₁₋₄alkyl)₂, —SO₂NH₂, —SO₂NHC₁₋₄alkyl, or —SO₂N(C₁₋₄alkyl)₂. (Cmpd 6), or a pharmaceutically acceptable salt, solvate, hydrate, prodrug, or derivative thereof, or a pharmaceutical composition thereof, and
a β-arrestin-biased β-blocker such that the high blood pressure or heart disease is treated and/or prevented in the subject.

11. The method according to claim 10, wherein the compound according to Formula (I) is cmpd 6, having the formula 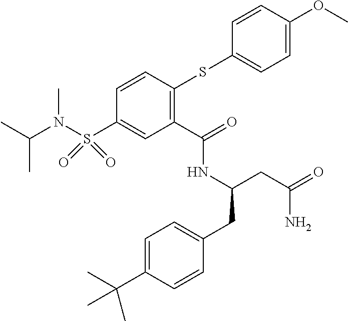 [SMILES: COc1ccc(Sc2ccc(S(=O)(=O)N(C)C(C)C)cc2C(=O)N[C@@H](CC(N)=O)Cc2ccc(C(C)(C)C)cc2)cc1]
or a pharmaceutically acceptable salt, solvate, hydrate, prodrug, or derivative thereof, or a pharmaceutical composition thereof.

12. The method according to claim 10, wherein the compound according to Formula (I) is analog A9, having the formula 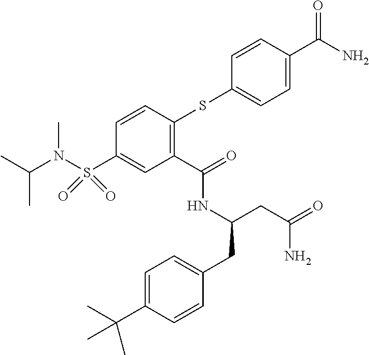 [SMILES: CC(C)N(C)S(=O)(=O)c1ccc(Sc2ccc(C(N)=O)cc2)c(C(=O)N[C@@H](CC(N)=O)Cc2ccc(C(C)(C)C)cc2)c1]
or a pharmaceutically acceptable salt, solvate, hydrate, prodrug, or derivative thereof, or a pharmaceutical composition thereof.

13. The method of any one of claims 10-12, wherein the beta-arrestin-biased beta blocker is carvedilol.

14. The method of any one of claims 10-12, wherein the compound according to Formula (I) is administered after the beta-arrestin-biased beta blocker.

15. The method of any one of claims 10-12, wherein the compound according to Formula (I) is administered concurrently with the beta-arrestin-biased beta blocker.

16. The method of any one of claims 10-12, wherein the compound according to Formula (I) is administered prior to the beta-arrestin-biased beta blocker.

17. A pharmaceutical composition comprising a compound according to Formula (I): 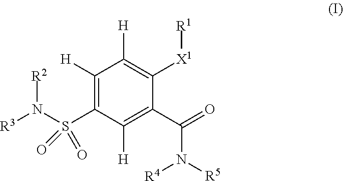
wherein:
R¹ is an aryl group optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆ alkyl)₂ —OC₃₋₆cycloalkyl, —NHC₃₋₆cycloalkyl, —N(C₁₋₆alkyl)(C₃₋₆cycloalkyl), and —N(C₃₋₆cycloalkyl)₂, and optionally the aryl is phenyl wherein two substituents join to form a 5- to 7-membered non-aromatic fused ring containing 1-2 heteroatom groups selected from NR^{1a} and O;
X₁ is O, N(H), N(C₁₋₄alkyl), S, S(O), S(O)₂, C(O), or CR^{1b}R^{1c};
R^{1a} is H or C₁₋₄alkyl;
R^{1b} and Ric are each independently hydrogen or C₁₋₄alkyl, or R^{1b} and Ric together with the carbon to which they are attached form a C₃₋₆cycloalkyl ring;
R² is hydrogen, C₁₋₆alkyl, or C₃₋₇cycloalkyl;
R³ is hydrogen, C₁₋₆alkyl, C₃₋₇cycloalkyl, or aryl, the aryl being optionally substituted with 1-5 substituents independently selected from the group consisting of C₁₋₆alkyl, C₁₋ₑhaloalkyl, C₃₋₇cycloalkyl, halogen, cyano, —OH, —OC₁₋₆alkyl, —OC₁₋₆haloalkyl, —NH₂, —NHC₁₋₆alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆cycloalkyl)₂; alternatively, R² and R³ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R⁴ is hydrogen, C₁₋₆alkyl, or C₃₋₇cycloalkyl;
R⁵ is CHR^{5a}R^{5b}; alternatively, R⁴ and R⁵ together with the nitrogen to which they are attached form a 4- to 8-membered heterocyclic ring optionally containing one additional heteroatom selected from N, O, and S, and being optionally substituted with 1-4 substituents independently selected from the group consisting of C₁₋₆alkyl, C₁₋₆haloalkyl, halogen, cyano, —OH, oxo, —OC₁₋₆alkyl, —NH₂, —NHC₁₋₆alkyl, and —N(C₁₋₆alkyl)₂;
R^{5a} is aryl or —C₁₋₃alkylene-aryl, wherein each aryl in R^{5a} is optionally substituted with 1-5 substituents independently selected from the group consisting of C₁₋₆alkyl, C₁₋₆haloalkyl, C₃₋₇cycloalkyl, halogen, cyano, —OH, —OC₁₋₆alkyl, —OC₁₋₆haloalkyl, —NH₂, —NHC₁₋₆ alkyl, —N(C₁₋₆alkyl)₂, —OC₃₋₆cycloalkyl, —NHC₃₋₆cycloalkyl, —N(C₁₋₆alkyl)(C₃₋₆cycloalkyl), and —N(C₃₋₆ cycloalkyl)₂;
R^{5b} is X² or —C₁₋₃alkylene-X²; and
X² is —CN, —C(O)OH, —C(O)OC₁₋₄alkyl, —C(O)NH₂, —C(O)NHC₁₋₄alkyl, —C(O)N(C₁₋₄alkyl)₂, —SO₂NH₂, —SO₂NHC₁₋₄alkyl, or —SO₂N(C₁₋₄alkyl)₂ (Cmpd 6), or a pharmaceutically acceptable salt, solvate, hydrate, prodrug, or derivative thereof, and
a β-arrestin-biased β-blocker or a pharmaceutically acceptable salt, solvate, hydrate, prodrug, or derivative thereof.

18. The pharmaceutical composition of claim 17, wherein the compound according to Formula (I) is selected from the group consisting of Cmpd 6, Cmpd A9, combinations thereof, pharmaceutically acceptable salts, solvates, hydrates, prodrugs, and derivatives thereof.

19. The pharmaceutical composition of claim 17, wherein the compound according to Formula (I) is Cmpd 6 having the formula 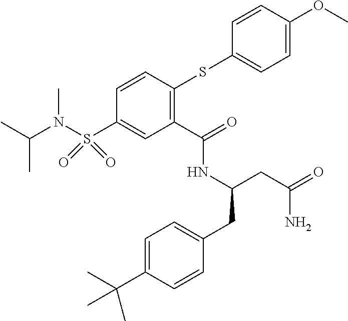 [SMILES: COc1ccc(Sc2ccc(S(=O)(=O)N(C)C(C)C)cc2C(=O)N[C@@H](CC(N)=O)Cc2ccc(C(C)(C)C)cc2)cc1]
or a pharmaceutically acceptable salt, solvate, hydrate, prodrug, or derivative thereof, or a pharmaceutical composition thereof.

20. The pharmaceutical composition of claim 17, wherein the compound according to Formula (I) is Cmpd A9 having the formula 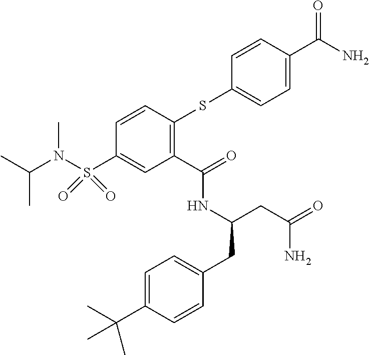 [SMILES: CC(C)N(C)S(=O)(=O)c1ccc(Sc2ccc(C(N)=O)cc2)c(C(=O)N[C@@H](CC(N)=O)Cc2ccc(C(C)(C)C)cc2)c1]
or a pharmaceutically acceptable salt, solvate, hydrate, prodrug, or derivative thereof, or a pharmaceutical composition thereof.

21. The pharmaceutical composition of any one of claims 17-20, wherein the β-arrestin-biased β-blocker is carvedilol.

22. All that is described and illustrated herein.

23. Any and all methods, processes, devices, systems, devices, kits, products, materials, compositions and/or uses shown and/or described expressly or by implication in the information provided herewith, including but not limited to features that may be apparent and/or understood by those of skill in the art.
